SEQUENCE LISTING (171 sequences)

SEQ ID NO: 1 - RNA, 52 nt, Artificial
  hairpin ribozyme
  misc_feature (1)..(52): n = A, U, C or G
nnnnnnnnagaannnnaccagagaaacacacguugugguauauuaccuggua

SEQ ID NO: 2 - RNA, 16 nt, Homo sapiens
  misc_feature (1)..(16): n = A, U, C or G
nnnnngucnnnnnnnn

SEQ ID NO: 3 - RNA, 38 nt, Artificial
  Hammerhead ribozyme
  misc_feature (1)..(38): n = A, U, C or G
nnnnnnnncugaugannnnnnnnnnnngaaannnnnnn

SEQ ID NO: 4 - RNA, 17 nt, Homo sapiens
  misc_feature (1)..(17): n = A, U, C or G
  misc_feature (9)..(9): h = A, U or C
nnnnnnnuhnnnnnnnn

SEQ ID NO: 5 - DNA, 28 bp, Artificial
  Chimeric hammerhead ribozyme
  misc_feature (1)..(5): DNA
  misc_feature (23)..(28): DNA
  modified_base (6)..(7): 2′ O methyl
  modified_base (12)..(12): 2′ O methyl
  modified_base (14)..(16): 2′ O methyl
  modified_base (19)..(19): 2′ O methyl
  modified_base (21)..(22): 2′ O methyl
  misc_feature (15)..(16): propanediol linkers
tcttcaucugaugagcgaaaccggccag

SEQ ID NO: 6 - DNA, 28 bp, Artificial
  Chimeric hammerhead ribozyme
  misc_feature (1)..(5): DNA
  misc_feature (23)..(28): DNA
  modified_base (6)..(7): 2′ O methyl
  modified_base (12)..(12): 2′ O methyl
  modified_base (14)..(16): 2′ O methyl
  modified_base (21)..(22): 2′ O methyl
  misc_feature (15)..(16): propanediol linker
ccagacacugaugagcgaaaccagctaa

SEQ ID NO: 7 - RNA, 16 nt, Homo sapiens
  misc_feature (1)..(16): n = G, U, C or A
  misc_feature (1)..(16): n = A, U,  C or G
nnnnngucnnnnnnnn

SEQ ID NO: 8 - RNA, 16 nt, Homo sapiens
  misc_feature (1)..(16): n = A, U, C or G
nnnnnguannnnnnnn

SEQ ID NO: 9 - RNA, 18 nt, Artificial
  Ribozyme substrate binding sequence
  misc_feature (1)..(18): n = A, U, C or G
nnnnnnnnnnagaannnn

SEQ ID NO: 10 - RNA, 18 nt, Artificial
  Ribozyme substrate binding sequence
  misc_feature (1)..(18): n = A, U, C or G
nnnnnnnnnncgaannnn

SEQ ID NO: 11 - DNA, 87 bp, Artificial
  Oligonucleotide primer
  misc_feature (1)..(87): n = A, T, C or G
cgcgtaccaggtaatataccacggaccgaagtccgtgtgtttctctggtnnnnttctnnnnnnnnggatcctgtttccgcccggttt

SEQ ID NO: 12 - DNA, 22 bp, Artificial
  Oligonucleotide primer
gtccgtggtatattacctggta

SEQ ID NO: 13 - DNA, 20 bp, Artificial
  Oligonucleotide primer
cgaaaccgggcggaaacagg

SEQ ID NO: 14 - DNA, 25 bp, Artificial
  Primer
ggcgggactatggttgctgactaat

SEQ ID NO: 15 - DNA, 24 bp, Artificial
  Primer
ggttatcacgttcgcctcacacgc

SEQ ID NO: 16 - DNA, 25 bp, Artificial
  Primer
ctgactccatcgagccagtgtagag

SEQ ID NO: 17 - DNA, 16 bp, Artificial
  Ribozyme substrate binding sequence
  misc_feature (5)..(5): n = T when DNA and U when RNA
ccagnccaagaagacc

SEQ ID NO: 18 - DNA, 16 bp, Artificial
  Ribozyme substrate binding sequence
  misc_feature (1)..(16): n = T when DNA and U when RNA
ncgnngngagaaagcc

SEQ ID NO: 19 - DNA, 16 bp, Artificial
  Ribozyme substrate binding sequence
  misc_feature (1)..(16): n = T when DNA and U when RNA
gncnncanagaaggcc

SEQ ID NO: 20 - DNA, 16 bp, Artificial
  Ribozyme substrate binding sequence
  misc_feature (1)..(16): n = T when DNA and U when RNA
nganccgnagaacana

SEQ ID NO: 21 - DNA, 16 bp, Artificial
  Ribozyme substrate binding sequence
  misc_feature (1)..(16): n = T when DNA and U when RNA
nangcngnagaaanaa

SEQ ID NO: 22 - DNA, 16 bp, Homo sapiens
  misc_feature (5)..(5): n = A, T, C or G
ggtcngtctggactgg

SEQ ID NO: 23 - DNA, 16 bp, Homo sapiens
  misc_feature (5)..(5): n = A, T, C or G
ggctngtccacaacga

SEQ ID NO: 24 - DNA, 16 bp, Homo sapiens
  misc_feature (5)..(5): n = A, T, C or G
ggccngtcatgaagac

SEQ ID NO: 25 - DNA, 16 bp, Homo sapiens
  misc_feature (5)..(5): n = A, T, C or G
tatgngtcacggatca

SEQ ID NO: 26 - DNA, 16 bp, Homo sapiens
  misc_feature (5)..(5): n = A, T, C or G
ttatngtcacagcata

SEQ ID NO: 27 - DNA, 3008 bp, Homo sapiens
  ref <308>: DATABASE ACCESSION NUMBER: GenBank AL096880
  ref <309>: DATABASE ENTRY DATE: 1999-07-23
gcgggtgaacgacgcttcgtgcggcccgtctggctgctacacataccaggtgagcagacacagcacggagatgctgcacaacctgaaccagcagcgcaaaaacggcgggcgcttctgcgacgtgctcttgcgggtaggcgacgagagcttcccagcgcaccgcgccgtgctggccgcctgcagcgagtactttgagtcggtgttcagcgcccagttgggcgacggcggagctgcggacgggggtccggctgatgtagggggcgcgacggcagcaccaggcggcggggccgggggcagccgggagctggagatgcacactatcagctccaaggtatttggggacattctggacttcgcctacacttcccgcatcgtggtgcgcttggagagctttcccgaactcatgacggccgccaagttcctgctgatgaggtcggttatcgagatctgccaggaagtcatcaaacagtccaacgtacagatcctggtaccccctgcccgcgccgatataatgctctttcgcccccctgggacctcggacttgggcttccctttggacatgaccaacggggcagccttggcagccaacagcaatggcatcgccggcagcatgcagccagaggaggaggcagctcgggcggctggtgcagccattgcaggccaagcctctttgcctgtgttacctggggtggaccgcttgcccatggtggctggacccctatccccccaactgctgacttccccattccccagtgtggcatccagtgcccctcccctgactggcaagcgaggccggggccgcccaaggaaggccaacctgctggactcaatgtttgggtccccagggggcctgagggaggcaggcatccttccatgcggtctatgtggtaaggtgttcactgatgccaaccggctccggcagcacgaggcccagcacggtgtcaccagcctccagctgggctacatcgaccttcctcctccgaggctgggtgagaatgggctacccatctctgaagaccccgacggcccccgaaagaggagccggaccaggaagcaggtggcttgtgagatctgcggcaagatcttccgtgatgtgtatcatcttaaccggcacaagctgtcccactctggggagaagccctactcctgccctgtgtgtgggttgcggttcaagagaaaagaccgcatgtcctaccatgtgcggtcccatgatgggtccgtgggcaagccttacatctgccagagctgtgggaaaggcttctccaggcctgatcacttgaacggacatatcaagcaggtgcacacttctgagcggcctcacaagtgtcagacctgcaatgcttcttttgccacccgagaccgtctgcgctcccacctggcctgtcatgaagacaaggtgccctgccaggtgtgtgggaagtacttgcgggcagcatacatggcagaccacctgaagaagcacagcgaggggcccagcaacttctgcagtatctgtaaccgagaaggccagaaatgctcacatcaggatccgattgagagctctgactcctatggtgacctctcagatgccagcgacctgaagacgccagagaagcagagtgccaatggctctttctcctgcgacatggcagtccccaaaaacaaaatggagtctgatggggagaagaagtacccatgccctgaatgtgggagcttcttccgctctaagtcctacttgaacaaacacatccagaaggtgcatgtccgggctctcgggggccccctgggggacctgggccctgcccttggctcacctttctctcctcagcagaacatgtctctcctcgagtcctttgggtttcagattgttcagtcggcatttgcgtcatctttagtagatcctgaggttgaccagcagcccatggggcctgaagggaaatgaggcagctgctgtgtccccacggaaacaaccatctggggactgctgggaaatgctgtgaatgcggagggaagtgatgtttgggttctgtagctgagagatttttattcatttttaactgccccccaaccccactccaactccttctccaccacccattctcccaatggtctttagaaatagattttcatctgatattctgcagaaatatcaatgagacttggtatgggacaggggcagaaaacactacataggcctccaaggcaaaaccagtcccagtttctttaatgggaagaagctggaattcctggtgctcaattcttagtgaccccaatcctatacccaaatctatgatattctgggacctcagtgattttggtcccctcccacttctctagttcgtcatcctcccttcccatatccttcaaaagaaccacactagggtctccacctacttatacaatgcggatgcccaactgtttttaaggaagccagaagcatcccatggaccatggggtgagtgtcctccaagagccccctgagctcagccctctgcctggagggctccagacctttctgagccctgcttggaggcgagcattttcactgctaggacaagctcagctgttgaggacacccccaccccaaatttcagttcttacgtgattttaaccattcaacatgctgttgggttttaattctctaattattattattattgttattattttttaggaccagttgtagtgaattgctactgaaagctatcccaggtgatacagagctctttgtaaaccgcagtcacacattagggttagtattaaactttgtttagatgtaccataattaacttggctagttgattgtttgaagtctatggaagaaatagttttatgcaaaattttaaaaaatgccagtctggtcagggaagtagggggtttcaatgctgttgggaaccaggaaggtgggacagccggcaggtagggacattgtgtacctcagttgtgtcacatgtgagcaagcccaggttgaccttgtgatgtgaattgatctgatcagactgtattaaaaatgttagtacattactcta

SEQ ID NO: 28 - DNA, 2551 bp, Mus musculus
  ref <308>: DATABASE ACCESSION NUMBER: GenBank AB029397
  ref <309>: DATABASE ENTRY DATE: 2001-07-17
ggcgtgtgcgctgggcgcacctgagagtactaccgagccctgggccgcacgtgcggggagtgtgagtggtccgctgcaaaaggcgtctcgctgcctaagggaagggaccagggactggcgggtgcccggcccggcgagtggggaaggggcagcgaccatggagcgggtcaacgacgcttcttgcggtccgtcgggctgctacacctaccaggtgagcagacacagtacggagatgctgcacaacctgaaccaacaacgcaaaaacggcgggcgcttttgcgacgtgctcctacgagtaggcgacgagagcttcccagcgcaccgcgccgtactggctgcctgcagcgagtactttgagtctgtgttcagcgcccagttaggcgacggcggagctgcagatggtggtcctgctgatgtgggaggcgcggcggcggctccaggcggcggagctggaggcagccgcgaactggagatgcacaccatcagttccaaagtgttcggagacatcctggacttcgcttatacgtcccgaatcgttgtgcgcctagagagcttccccgagctcatgacggccgccaagttcctgctgatgaggtcggtcatcgagatctgccaggaagtaatcaaacagtccaacgtgcagatcctcgtgccccctgcccgggctgatatcatgctctttcgcccacctgggacttctgacttgggcttccctttggacatgaccaacggggcagccatggcagccaacagtaacggtattgctggcagtatgcagcccgaggaggaggctgccagggccacaggtgctgctattgcgggccaagcttctctgcctgtgttacccggggtggacagattgcccatggtggctggacccctatccccccaactactgacttctccgttccctaatgtggcatccagtgcacctccactaactagcaagcgaggccggggacgccccaggaaggccaacctgctggactccatgtttgggtctccagggggcttgagggaagcaggcatccttccatgtggcctgtgcgggaaggtgttcactgacgccaaccggctccggcaacatgaggcccagcacggcgtcacaagcctccagttgggctatatcgatcttcctcctccaaggctgggtgagaatgggttacccatctccgaggaccccgatggccccagaaaaaggagccggaccagaaagcaagtggcttgtgagatctgtggcaagatctttcgtgacgtataccatctcaaccggcataagctttcccactcgggggagaagccgtactcgtgcccggtgtgtggtctgcggttcaagagaaaagaccgaatgtcgtaccatgtgaggtcccatgatgggtcagtgggcaaaccgtacatctgccagagctgtgggaaaggtttctccaggccagatcacttgaatggacatatcaagcaggtgcacacttctgagcgacctcacaagtgtcagacctgcaatgcctcctttgcgactcgagaccgcctgcgctcccacctggcctgtcatgaagacaaggtgccctgccaggtgtgtggaaagtacttgcgggccgcatacatggcagaccacctgaagaagcacagtgaggggcccagcaacttctgtagcatctgtaaccgagaaggccagaaatgctcacatcaggatctgattgagagctccgactcctacggtgacctctccgacgccagcgacctgaagacgccagagaaacagagtgccaacggctccttctcctgcgacgtggcagtccctaaaaacaaaatggagtctgacggggagaagaagtacccatgccctgaatgtgggagcttcttccgttctaagtcctacttgaacaaacacatccagaaggtgcatgtccgtgcccttgggggtcccttgggggacctgggccctgcccttggttcacctttctctccccagcagaacatgtctcttcttgagtcctttggatttcagattgttcagtcagcgtttgcatcatctttagtggatcctgaggtggaccagcagcccatggggcctgaagggaagtgagacatgctgtgtccccacagaacagccgctggggactgctgagcaatgctgtgaatgcagagggaagtgatgtctttgggttctgtagctgagagatttttattcatttttaaaccccatccctgcccctccctccctccattaaccccacctgctgtggtcttagaaacagactctcatctgatactctctgtagaaatactgagagacccaatatggggcaagggcagaaagcactacatcggcctctgaggcagtaccagttctggtttctctagtggtctgaagccagaatttctggtgctcaagttattggtggcctcaccccaacttttaaggcactcttggcctcgcttggtaagttttggtcccacttctttcattcctcacccccctgcctctgtcctcaaaagaagttaagtagggtctctaccctcttatggccaactatttttaaggaagccagaa

SEQ ID NO: 29 - DNA, 3038 bp, Homo sapiens
  ref <308>: DATABASE ACCESSION NUMBER: GenBank AF119256
  ref <309>: DATABASE ENTRY DATE: 2000-03-13
gggcctactctgccgccgccgccgcccgcccgctccagccgccgccgccgccgccaccgccctccaggctccgggacccggcccgcgccaccgcccccgtgcgcgccccgccgccgccgccttcgccttcgccttttgtttcctccgctccggcgcccccgccccggctcgcgctttgcaggggacgcagcgcgcgcccccagcgggcccgggaaaagccgcggcgcgcgcgcgcgcctgcgcggcggacccctccttctcctccccgcgtgcgcgtgcccttcttggctgcgcgccggcgccgcctggcgggcgggaggggaggtggcaggcgcgtttgcaggaggggcgcacctcttcgctcgcgcacccccccggaaggtagaccgggaaggggaagcgggcgggcggagaggagagatggcgcgcagtccagcgagggcgggggttggctatgtggggggtggtgcaccccgcagtctagacagtctgatccgggctgggggcgtgtacactcggcgcacctgcgagactacagagcctcgggccggcacgtgtggggagtgtggacacgtctgctgcgccccgcttctcgctgctgaggggaagggagggggcgggcaggtgcagcggccgggctagtgggagggggcggcggccatggagcgggtgaacgacgcttcgtgcggcccgtctggctgctacacataccaggtgagcagacacagcacggagatgctgcacaacctgaaccagcagcgcaaaaacggcgggcgcttctgcgacgtgctcttgcgggtaggcgacgagagcttcccagcgcaccgcgccgtgctggccgcctgcaacgagtactttgagtcggtgttcagcgcccagttgggcgacggcggagctgcggacgggggtccggctgatgtagggggcgcgacggcagcaccaggcggcggggccgggggcagccgggagctggagatgcacactatcagctccaaggtatttggggacattctggacttcgcctacacttcccgcatcgtggtgcgcttggagagctttcccgaactcatgacggccgccaagttcctgctgatgaggtcggttatcgaaatctgccaggaagtcatcaaacagtccaacgtacagatcctggtaccccctgcccgcgccgatataatgctctttcgcccccctgggacctcggacttgggcttccctttggacatgaccaacggggcagccttggcagccaacagcaatggcatcgccggcagcatgcagccagaggaggaggcagctcgggcggctggtgcagccattgcaggccaagcctctttgcctgtgttacctggggtggaccgcttgcccatggtggctggacccctatccccccaactgctgacttccccattccccagtgtggcatccagtgcccttcccctgactggcaagcgaggccggggccgcccaaggaaggccaacctgctggactcaatgtttgggtccccagggggcctgagggaggcaggcatccttccatgcggtctatgtggtaaggtgttcactgatgccaaccggctccggcagcacgaggcccagcacggtgtcaccagcctccagctgggctacatcgaccttcctcctccgaggctgggtgagaatgggctacccatctctgaagaccccgacggcccccgaaagaggagccggaccaggaagcaggtggcttgtgagatctgcggcaagatcttccgtgatgtgtatcatcttaaccggcacaagctgtcccactctggggagaagccctactcctgccctgtgtgtgggttgcggttcaagaaaaaagaccgcatgtcctaccatgtgcggtcccatgatgggtccgtgggcaagccttacatctgccagagctgtgggaaaggcttctccaggcctgatcacttgaacggacatatcaagcaggtgcacacttctgagcggcctcacaagtgtcaggtgtgggttgggagcagcagcggcctgccgcccctggaacctcttcctagcgacctgccatcatgggactttgcccagcctgctttgtggaggtcgtcccattcggttcctgacaccgccttttccctttctctaaaaaaatcattcccagcccttgaaaacctgggcccagcacactccagcaacactctcttctgcccagccccgccgggatatctgaggcagggctggactaccccagagggcagcagggcctttacccagtggcctgttggctagcctgggcctccctggagagggttgacagtggaagggaacaggaggggcatttggcctgagacccctgcttttgggaaaggctagcagggtggttcctgcccagcatgcccagctcctccctgggtgactcggaatctttcccatgtcaaaacccccaaatgggggtacaaagaacacctttctggaaccccctataacatccaaatttctttctgggctctcttgccttttcccccctttcacaaatggcacccctgggcatctgtccttgcctaagttattttggaagttggtgccttcctgggaactaaccaccaacttatctgcttcccttcccctggcatcacttcccataagcctggggtttctaaactggggcctggccacccctttccccactccaagagtgagtcggcctccagagaagactggcacaattccaactagagtcaacccatgctgccttttgcccttcccattcagatttagatcctgctttcatttttggctagtgaagtagatttttgtgtttttgaggtttattagcaggtttgctcaggaaccaaattaatgagtagttttatattgggccaccccaatatatggctttgggggctgaaaaagcagatgtagaccccctccctcggatccttattggtgtgccctttagcattccgcagattttgcggggtgaacaggagtgatgataaaatttttcattttaacaaaaaaaaaaaaaaaaaaaaaaaaaaaaaaaaaaaaaa

SEQ ID NO: 30 - DNA, 441 bp, Homo sapiens
  misc_feature (350)..(428): n = A, T, C or G
  ref <308>: DATABASE ACCESSION NUMBER: GenBank H09317
  ref <309>: DATABASE ENTRY DATE: 1995-06-23
tttttttttttttcttggttttgtaggcatttatttacatcatatttcaatacttcagaagcttaaacagtgtcaggggtatagcagttctgagaaacagttttacaagaagacataaactaaggggtacccatgagtgcgtctcatccttcctctcccaggccagagtaacaggtatgctgagatgctcttgcccttggccccggggtgctcacctccagcctcgagctgcctcacccagttagccagggggctgcacaggtgtttgcgtgtcctacatgtggcctgtcatgaagaaggtccgcatacgtggctctaggctgtgcagggcaagtcttcccaagggactnaaggaagtcaccctgaaatcctctccccatgagggacctcttcctaagtcagattttctcactgctcctngttccagntcctgttgccatt

SEQ ID NO: 31 - DNA, 396 bp, Homo sapiens
  misc_feature (1)..(396): n = A, T, C or G
  ref <308>: DATABASE ACCESSION NUMBER: GenBank R12420
  ref <309>: DATABASE ENTRY DATE: 1995-04-12
gcatcacacacacagtgacttttgtgtttgatcttgaagaatgagcgaaccaggcagggagtcggggaggagaatcgcattccttgaggaaagagcagcatgtgggaaaacataaatgcacgcaataacctggctcacatgttaagagaactttctgactataatgagggatgtgttgctgccccaagctttcattatncttaaggagtttgtttgaacactctctagaggctttttaataataggattgttttagctggtctgtctggactggttagatataacactatttttaaatgaccccantcttcatttacatttgttgaagntttcccatttttttttnagggttaccgtaaggganttttttggggaccccanaaattctttggcatt

SEQ ID NO: 32 - DNA, 953 bp, Homo sapiens
  ref <308>: DATABASE ACCESSION NUMBER: GenBank BG116747
  ref <309>: DATABASE ENTRY DATE: 2001-01-29
atatctaagcagttataataggttgattttgtaatttaaaaaatgtaaaaatgcatacatgcactagtgcatgaatggcagccaggatgagtggaattggagaagcatcacacacacagtgactttgtgtttgatcttgaagaatgagcgaaccaggcagggagtcggggaggagaatcgcattccttgaggaaagagcagcatgtgggaaaacataaatgcacgcaataacctggctcacatgttaagagaactttctgactataatgagggatgtgttgctgccccaagcttcattatctaaggagtttgttgaacactctctagaggcttttaataataggattgtttagctggtctgtctggactggttagatataacactatttaaatgacccaatctcattacattgtgaagatttccattttttaggttacgtaagaaattttggacctaaaaatcttgcattttaagacagtcttgtcagaattacttttggctctaaatgaattctgtaacatttgtattctaaattgacctttagtaaaagcaggaatggccatattcaaactggtaacctcgcaaatcctgccaccctttcactttctgtctcaatacattgatgtcctctaaccatttctgtcttatgtgggctttagtgccacttatcaaaattgtgtgcaagttcctggctacagtaacagtttttgtctgggttgtctagacagggaattctgcctgaggtcatcatttttgtgactggtacttgagggctcgatgttactgcatgataagaggggtctgggggtgcgcaatcgccttttgcaattggatacccattagcggggcatctcgttcataacccaaaagcaatcaaatttatggtcttagtgggctcaaagcgcggggtttttaccaaaaaagagactggacccttggtaagcctcggaca

SEQ ID NO: 33 - DNA, 910 bp, Homo sapiens
  ref <308>: DATABASE ACCESSION NUMBER: GenBank BG115920
  ref <309>: DATABASE ENTRY DATE: 2001-01-29
ttcatatatatctaagcagttataataggttgattttgtaatttaaaaaatgtaaaaatgcatacatgcactagtgcatgaatggcagccaggatgagtggaattggagaagcatcacacacacagtgacttttgtgtttgatcttgaagaatgagcgaaccaggcagggagtcggggaggagaatcgcattccttgaggaaagagcagcatgtgggaaaacataaatgcacgcaataacctggctcacatgttaagagaactttctgactataatgagggatgtgttgctgccccaagcttcattatctaaggagttgttgaacactctctagaggcttttaataataggattgtttagctggtctgtctggactggttagatataacactatttaaatgacccaatctcattacatgtgaagatttccattttttaggttacgtaagaaatttggacctaaaaatcttgcattttaagacagtcttgtcagaattacttttggctctaaatgaattctgtaacatttgtattctaaattgacctttagtaaaagcaggaatggcatattcaactggtaacctcgcaaatcctgcccacctttcactttctgtctcaatacatttggatgtcctccttaacccatttcctgtcttatgtgggtttaggttgccacttattcaaaattgtggtgcaaatttccttgcttaccagtaacaggtcttggctgggggctggtaagcagggggatttctgcgcggtcacacttttggtggtgggtacttgaaggggtcaacgaggagaataggaaagagggtgggggggggaaaaatatttggaaggagaacacatacggggggacggtggcactacaagagcgagcaagaaatggaataaggaggtgaaaaag

SEQ ID NO: 34 - DNA, 952 bp, Homo sapiens
  ref <308>: DATABASE ACCESSION NUMBER: GenBank BG111236
  ref <309>: DATABASE ENTRY DATE: 2001-01-29
attttttgaggatttactattgccagacactgtgctacaagctgggaatgctgacagtataagataaagagggaaatgatgggggatgggtcatgtaaagggagaactttcatttttacttcatatatatctaagcagttataataggttgatttttgtaatttaaaaaatgtaaaaatgcatacatgcactagtgcatgaatggcagccaggatgagtggaattggagaagcatcacacacacagtgacttttgtgtttgatcttgaagaatgagcgaaccaggcagggagtcggggaggagaatcgcattccttgaggaaagagcagcatgtgggaaaacataaatgcacgcagtaacctggctcacatgttaagagaactttctgactataatgagggatgtgttgctgccccaagcttcattatctaaggagtttgttgaacactctctagaggcttttaataataggattgtttagctggtctgtctggactggttagatataacactatttaaatgacccaatctcattacattgtgaagatttccatttttgcacggttacgtaaagaaattttggaccctaaaatcttgcattttaagacagctctttgtcaagaattactttttggctctaaatgaattcctgtaacatttggacttctaatgaccttaggtacagagcaggatggcctttcaatctgttacctcggaatgctgccacccttcactatctggcttcatcatggtgtcctctacccatccctgcttgtggttagcgcatttagtatggtcgagtcttgcgacatacgcttgctggctctccctgtgcccatccttggacgcgacgctcgtactagccaggctgcgcggaacctagggcatacggcggacagaacaatgtgatgtcaacgtaccagcctcagtatgtgtcctgcgtgtgtatagc

SEQ ID NO: 35 - DNA, 857 bp, Homo sapiens
  misc_feature (555)..(595): n = A, T, C or G
  ref <308>: DATABASE ACCESSION NUMBER: GenBank BF692624
  ref <309>: DATABASE ENTRY DATE: 2000-12-21
gataaagagggaaatgatgggggatgggtcatgtaaagggagaactttcatttttacttcatatatatctaagcagttataataggttgattttgtaatttaaaaaatgtaaaaatgcatacatgcactagtgcatgaatggcagccaggatgagtggaattggagaagcatcacacacacagtgactttgtgtttgatcttgaagaatgagcgaaccaggcagggagtcggggaggagaatcgcattccttgaggaaagagcagcatgtgggaaaacataaatgcacgcaataacctggctcacatgttaagagaactttctgactataatgagggatgtgttgctgccccaagcttcattatctaaggagttgttgaacactctctagaggcttttaataataggattgtttagctggtctgtctggactggttagatataacactatttaaatgacccaatctcattacattgtgaagatttccattttttaggttacgtaagaaatttggacctaaaaatcttgcattttacgacagtctttgtcagaatnactttttggtctaaatgaattctgtaacattgtatctaanattgacctttagtaaaagcaggaatggccattttcaaactggttaacctcggagatctgcaacctttcaatttctgtctcatacattgatgttctctaaccatttctgtttatgggtttagtgccctttcaacatggtgccacttccctgcgcacagacacgcgttggtggctggtacagggacctcgcggtgcaccatggacggtcagcgccagataacaaaagggggggcaacacacgcaggcacccaagggcgcacaca

SEQ ID NO: 36 - DNA, 846 bp, Homo sapiens
  ref <308>: DATABASE ACCESSION NUMBER: GenBank BE892951
  ref <309>: DATABASE ENTRY DATE: 2000-09-26
gatttttgtaatttaaaaaatgtaaaaatgcatacatgcactagtgcatgaatggcagccaggatgagtggaattggagaagcatcacacacacagtgacttttgtgtttgatcttgaagaatgagcgaaccaggcagggagtcggggaggagaattgcattccttgaggaaagagcagcatgtgggaaaacataaatgcacgcaataacctggctcacatgttaagagaactttctgactatatgagggatgtgttgctgccccaagcttcattatctaaggagtttgttgaacactctctagaggcttttaataataggattgtttagctggtctgtctggactggttagatataacactatttaaatgacccaatctcattacattgtgaagatttccattttttaggttacgtaagaaattttggacctaaaaatcttgcattttaagacagtctttgtcagaattactttttggctctaaatgaattctgtaacatttgtattctaaattgacctttataaaagcaggaatggccatattcaaactggtaacctcgcaaatcctgcccaccctgttcacttttctgttctccataacattggatgtcccttcttaacccatttcctgttcttatgttggctttaggtgccacttatccaaacttggtgtgcaaatttccctttggctaacagctcaaccaggtttttggtctgggccttgtcttagccctgggattctgcccggagttcctcacttcttggtgaccgggcctgagtgctccgcgcggttattccgcgaaaaaaggccttgcggggggcacaaccctttctggagggtcccc

SEQ ID NO: 37 - DNA, 462 bp, Homo sapiens
  ref <308>: DATABASE ACCESSION NUMBER: GenBank AL138059
  ref <309>: DATABASE ENTRY DATE: 2000-03-01
ggattgtttagctggtctgtctggactggttagatataacactatttaaatgacccaatctcattacattgtgaagatttccattttttaggttacgtaagaaattttggacctaaaaatcttgcattttaagacagtctttgtcagaattactttttggctctaaatgaattctgtaacatttgtattctaaattgacctttagtaaaagcaggaatggccatattcaaactggtaacctcgcaaatcctgcccaccctttcactttctgtctcaatacattgatgtcctctaacccatttcctgtcttatgtggctttagtgccacttatcaaaattgtgtgcaaatttccttggctaacagtaacagtttttgtctgggcttgtctagcagtggaattctgcctgagttcatcatttttgtgactggtacttgaagtgcatcagatgattaatttcatg

SEQ ID NO: 38 - DNA, 384 bp, Homo sapiens
  misc_feature (6)..(8): n = A, T, C or G
  ref <308>: DATABASE ACCESSION NUMBER: GenBank AI754258
  ref <309>: DATABASE ENTRY DATE: 1999-06-22
ggcccnanggggtaccgggcccccccgtttgttgaacactctctagaggcttttaataataggattgtttagctggtctgtctggactggttagatataacactatttaaatgacccaatctcattacattgtgaagatttccattttttaggttacgtaagaaattttggacctaaaaatcttgcattttaagacagtctttgtcagaattactttttggctctaaatgaattctgtaacatttgtattctaaattgacctttagtaaaagcaggaatggccatattcaaactggtaacctcgcaaatcctgcccaccctttcactttctgtctcaatacattgatgtcctctaacccatttcctgtcttatgtggctttagt

SEQ ID NO: 39 - DNA, 423 bp, Homo sapiens
  ref <308>: DATABASE ACCESSION NUMBER: GenBank AA495929
  ref <309>: DATABASE ENTRY DATE: 1997-08-11
gaagaatgagcgaaccaggcagggagtcggggaggagaattgcattccttgaggaaagagcagcatgtgggaaaacataaatgcacgcaataacctggctcacatgttaagagaactttctgactataatgagggatgtgttgctgccccaagcttcattatctaaggagtttgttgaacactctctagaggcttttaataataggattgtttagctggtctgtctggactggttagatataacactatttaaatgacccaatctcattacattgtgaagatttccattttttaggttacgtaagaaattttggacctaaaaatcttgcattttaagacagtctttgtcagaattactttttggctctaaatgaattctgtaacatttgtattctaaattgacctttagtaaaagcaggaatg

SEQ ID NO: 40 - DNA, 1555 bp, Homo sapiens
  ref <308>: DATABASE ACCESSION NUMBER: GenBank AK025818
  ref <309>: DATABASE ENTRY DATE: 2000-09-29
aaaaatgtaaaaatgcatacatgcactagtgcatgaatggcagccaggatgagtggaattggagaagcatcacacacacagtgacttttgtgtttgatcttgaagaatgagcgaaccaggcagggagtcggggaggagaatcgcattccttgaggaaagagcagcatgtgggaaaacataaatgcacgcaataacctggctcacatgttaagagaactttctgactataatgagggatgtgttgctgccccaagcttcattatctaaggagtttgttgaacactctctagaggcttttaataataggattgtttagctggtctgtctggactggttagatataacactatttaaatgacccaatctcattacattgtgaagatttccattttttaggttacgtaagaaattttggacctaaaaatcttgcattttaagacagtctttgtcagaattactttttggctctaaatgaattctgtaacatttgtattctaaattgacctttagtaaaagcaggaatggccatattcaaactggtaacctcgcaaatcctgcccaccctttcactttctgtctcaatacattgatgtcctctaacccatttcctgtcttatgtggctttagtgccacttatcaaaattgtgtgcaaatttccttggctaacagtaacagtttttgtctgggcttgtctagcagtggaattctgcctgagttcatcatttttgtgactggtacttgaagtgcatcagatgattaatttcatgataagagggctttttggggtggtgaaatagacatttatggaaaatgggatacccacattaagcagggtgactacctgtttaccatacaacccacacaaagccaatacaactatagatgtgctttatttagtctgttgcctctgcaaacattgcccgtgtgtttctctatgcccttcaaaaacatcagagcagcacatcctggaagatcctatcttttgtaagtttaagaagcagcctcttgtcacaggttgactcctaggtagtgtgcctagtgaccaagagggctgctaagaaagctttctgaccacttgtggctgtcattggactgatttgcccagatgacatcaattgggaatttgaggcatgacctataaagatcagttgcttgcaagagtctcaggaaaataattgtggagttaagaaacttgaagcgatttttaaaaattacctaacccaaccttctcatttgaaaaattaaaaaataaataggccagatatggcagctcatgcctgtaatcccagcgctgtgggagcctgaggcgggtgggtctcttgaggccaggagttcaagaccagcctggacaacatggtgaaaccctgtctctactaaaaacacaaaaattagctgagtgtggtggcaggcgcctgtaatcccagctactcaggaggctaaggtgggaggattacttgaaccggggaggcataggttgtagtgagccaagatcgtgtcactgcactccagcctgggtgacagagtaagactctgcctaaaaaaaaaaaaaaaaaaa

SEQ ID NO: 41 - DNA, 2195 bp, Homo sapiens
  ref <308>: DATABASE ACCESSION NUMBER: GenBank NM_001895.1
  ref <309>: DATABASE ENTRY DATE: 2000-10-31
aggggagagcggccgccgccgctgccgcttccaccacagtttgaagaaaacaggtctgaaacaaggtcttacccccagctgcttctgaacacagtgactgccagatctccaaacatcaagtccagctttgtccgccaacctgtctgacatgtcgggacccgtgccaagcagggccagagtttacacagatgttaatacacacagacctcgagaatactgggattacgagtcacatgtggtggaatggggaaatcaagatgactaccagctggttcgaaaattaggccgaggtaaatacagtgaagtatttgaagccatcaacatcacaaataatgaaaaagttgttgttaaaattctcaagccagtaaaaaagaagaaaattaagcgtgaaataaagattttggagaatttgagaggaggtcccaacatcatcacactggcagacattgtaaaagaccctgtgtcacgaacccccgccttggtttttgaacacgtaaacaacacagacttcaagcaattgtaccagacgttaacagactatgatattcgattttacatgtatgagattctgaaggccctggattattgtcacagcatgggaattatgcacagagatgtcaagccccataatgtcatgattgatcatgagcacagaaagctacgactaatagactggggtttggctgagttttatcatcctggccaagaatataatgtccgagttgcttcccgatacttcaaaggtcctgagctacttgtagactatcagatgtacgattatagtttggatatgtggagtttgggttgtatgctggcaagtatgatctttcggaaggagccatttttccatggacatgacaattatgatcagttggtgaggatagccaaggttctggggacagaagatttatatgactatattgacaaatacaacattgaattagatccacgtttcaatgatatcttgggcagacactctcgaaagcgatgggaacgctttgtccacagtgaaaatcagcaccttgtcagccctgaggccttggatttcctggacaaactgctgcgatatgaccaccagtcacggcttactgcaagagaggcaatggagcacccctatttctacactgttgtgaaggaccaggctcgaatgggttcatctagcatgccagggggcagtacgcccgtcagcagcgccaatatgatgtcagggatttcttcagtgccaaccccttcaccccttggacctctggcaggctcaccagtgattgctgctgccaacccccttgggatgcctgttccagctgccgctggcgctcagcagtaacggccctatctgtctcctgatgcctgagcagaggtgggggagtccaccctctccttgatgcagcttgcgcctggcggggaggggtgaaacacttcagaagcaccgtgtctgaaccgttgcttgtggatttatagtagttcagtcataaaaaaaaaattataataggctgattttcttttttctttttttttttaactcgaacttttcataactcaggggattccctgaaaaattacctgcaggtggaatatttcatggacaaatttttttttctcccctcccaaatttagttcctcatcacaaaagaacaaagataaaccagcctcaatcccggctgctgcatttaggtggagacttcttcccattcccaccattgttcctccaccgtcccacactttagggggttggtatctcgtgctcttctccagagattacaaaaatgtagcttctcaggggaggcaggaagaaaggaaggaaggaaagaaggaagggaggacccaatctataggagcagtggactgcttgctggtcgcttacatcactttactccataagcgcttcagtggggttatcctagtggctcttgtggaagtgtgtcttagttacatcaagatgttgaaaatctacccaaaatgcagacagatactaaaaacttctgttcagtaagaatcatgtcttactgatctaaccctaaatccaactcatttatacttttatttttagttcagtttaaaatgttgataccttccctcccaggctccttaccttggtcttttccctgttcatctcccaacatgctgtgctccatagctggtaggagagggaaggcaaaatctttcttagttttctttgtcttggccattttgaattc

SEQ ID NO: 42 - DNA, 2013 bp, Homo sapiens
  ref <308>: DATABASE ACCESSION NUMBER: GenBank NM_032639
  ref <309>: DATABASE ENTRY DATE: 2001-08-06
ggcacgaggaggcgatggccctgctgctggtgctcctcgcctcttggggcctggggcagtgagggggccggcgggcgtgggccgagtggccgcgggcgccatggagggggtgctgtacaagtggaccaactatctgagcggttggcagcctcgatggttccttctctgtgggggaatattgtcctattatgattctcctgaagatgcctggaaaggttgcaaagggagcatacaaatggcagtctgtgaaattcaagttcattctgtagataatacacgcatggacctgataatccctggggaacagtatttctacctgaaggccagaagtgtggctgaaagacagcggtggctggtggccctgggatcagccaaggcttgcctgactgacagtaggacccagaaggagaaagagtttgctgaaaacactgaaaacttgaaaaccaaaatgtcagaactaagactctactgtgacctccttgttcagcaagtagataaaacaaaagaagtgaccacaactggtgtgtccaattctgaggagggaattgatgtgggaactttgctgaaatcaacctgtaatacttttctgaagaccttggaagaatgcatgcagatcgcaaatgcagccttcacctctgagctgctctaccgcactccaccaggatcacctcagctggccatgctcaagtccagcaagatgaaacatcctattataccaattcataattcattggaaaggcaaatggagttgagcacttgtgaaaatggatctttaaatatggaaataaatggtgaggaagaaatcctaatgaaaaataagaattccttatatttgaaatctgcagagatagactgcagcatatcaagtgaggaaaatacagatgataatataacagtccaaggtgaaataaggaaggaagatggaatggaaaacctgaaaaatcatgacaataacttgactcagtctggatcagactcaagttgctctccggaatgcctctgggaggaaggcaaagaagttatcccaactttctttagtaccatgaacacaagctttagtgacattgaacttctggaagacagtggcattcccacagaagcattcttggcatcatgttatgctgtggttccagtattagacaaacttggccctacagtgtttgctcctgttaagatggatcttgttggaaatattaagaaagtaaatcagaagtatataaccaacaaagaagagtttaccactctccagaagatagtgctgcacgaagtggaggcggatgtagcccaggttaggaactcagcgactgaagccctcttgtggctgaagagaggtctcaaatttttgaagggatttttgacagaagtgaaaaatggggagaaggatatccagacagccctaagaaatccaacagaaaacacttgacaccaaaacataccctgatgaagatcctgaacttcaagaatgaagaaagaattcctcaccattcaggcagaaaaagcaagtcaccaagggacctcaaacttcctttccacaagattctgtgacgggaaacaatgggggagtatttccgaagttctgagtaggaaaaaagaatgactcaaatgtattattgccaaccaagtcgtcaaatctaatgtcaagttctcttaagcaggtaagaactcagaacataatacctgagtgccttcttaaggaaaccatttgataggaaagatgaaccaaataactcaatgatggatgagctggtagaaaaaaagctggtggtgaaccaaggtcaaactggaaattatagtcacagtatagatatagattataaatattacaaaccctaagatagctaataaattgggaatgggagaagggaggatataagagcactaatgccctcttattttcatagcagagacttgatactgtctcaacttttttcaaaaacacaatttcttaaattttttggtaatcttttaaataaacagatttctaaaaagaaaaaaaaaaaaaaaaaaaaaaaa

SEQ ID NO: 43 - DNA, 16 bp, Artificial
  Ribozyme substrate binding sequence
agcagccaagaaggcc

SEQ ID NO: 44 - DNA, 16 bp, Artificial
  Ribozyme substrate binding sequence
  misc_feature (1)..(1): n = T when DNA and U when RNA
ngaacaccagaacaaa

SEQ ID NO: 45 - DNA, 16 bp, Artificial
  Ribozyme substrate binding sequence
  misc_feature (2)..(2): n = T when DNA and U when RNA
ancagccgagaacccg

SEQ ID NO: 46 - DNA, 16 bp, Artificial
  Ribozyme substrate binding sequence
  misc_feature (6)..(16): n = T when DNA and U when RNA
ccccancaagaaccan

SEQ ID NO: 47 - DNA, 16 bp, Artificial
  Ribozyme substrate binding sequence
acccaaagagaacgag

SEQ ID NO: 48 - DNA, 16 bp, Artificial
  Ribozyme substrate binding sequence
  misc_feature (5)..(5): n = T when DNA and U when RNA
caaangccagaagaac

SEQ ID NO: 49 - DNA, 16 bp, Homo sapiens
  misc_feature (1)..(16): n = T when DNA and U when RNA
anggagncngangggg

SEQ ID NO: 50 - DNA, 16 bp, Artificial
  Ribozyme substrate binding sequence
gtacgttgagaagttt

SEQ ID NO: 51 - DNA, 16 bp, Artificial
  Ribozyme substrate binding sequence
cccctgggagaacaaa

SEQ ID NO: 52 - DNA, 16 bp, Artificial
  Ribozyme substrate binding sequence
accacataagaagcat

SEQ ID NO: 53 - DNA, 16 bp, Artificial
  Ribozyme substrate binding sequence
aggctggtagaaccgt

SEQ ID NO: 54 - DNA, 16 bp, Artificial
  Ribozyme substrate binding sequence
cagagtggagaagctt

SEQ ID NO: 55 - DNA, 16 bp, Artificial
  Ribozyme substrate binding sequence
catcatggagaagcac

SEQ ID NO: 56 - DNA, 16 bp, Artificial
  Ribozyme substrate binding sequence
tgcccacgagaacatg

SEQ ID NO: 57 - DNA, 16 bp, Artificial
  Ribozyme substrate binding sequence
gcaggtctagaacttg

SEQ ID NO: 58 - DNA, 16 bp, Artificial
  Ribozyme substrate binding sequence
ggagcgcaagaagtct

SEQ ID NO: 59 - DNA, 16 bp, Artificial
  Ribozyme substrate binding sequence
tttttgggagaagcca

SEQ ID NO: 60 - DNA, 16 bp, Artificial
  Ribozyme substrate binding sequence
cgaggagaagaatgtt

SEQ ID NO: 61 - DNA, 16 bp, Artificial
  Ribozyme substrate binding sequence
ctaaagatagaacaaa

SEQ ID NO: 62 - DNA, 16 bp, Artificial
  Ribozyme substrate binding sequence
tccgtgggagaacagc

SEQ ID NO: 63 - DNA, 16 bp, Artificial
  Ribozyme substrate binding sequence
tttctaaaagaaattg

SEQ ID NO: 64 - DNA, 16 bp, Artificial
  Ribozyme substrate binding sequence
gaaactggagaaggtt

SEQ ID NO: 65 - DNA, 16 bp, Artificial
  Ribozyme substrate binding sequence
tgggagggagaaaaaa

SEQ ID NO: 66 - DNA, 16 bp, Artificial
  Ribozyme substrate binding sequence
gggaggatagaaaact

SEQ ID NO: 67 - DNA, 16 bp, Artificial
  Ribozyme substrate binding sequence
taggtggaagaactag

SEQ ID NO: 68 - DNA, 16 bp, Artificial
  Ribozyme substrate binding sequence
tcttggagagaactca

SEQ ID NO: 69 - DNA, 16 bp, Artificial
  Ribozyme substrate binding sequence
taatgtgtagaagcgg

SEQ ID NO: 70 - DNA, 16 bp, Artificial
  Ribozyme substrate binding sequence
cctgaccaagaaggca

SEQ ID NO: 71 - DNA, 16 bp, Artificial
  Ribozyme substrate binding sequence
acttccctagaaagac

SEQ ID NO: 72 - DNA, 16 bp, Artificial
  Ribozyme substrate binding sequence
tcacatgtagaacaac

SEQ ID NO: 73 - DNA, 16 bp, Homo sapiens
aaacagtccaacgtac

SEQ ID NO: 74 - DNA, 16 bp, Homo sapiens
tttgggtccccagggg

SEQ ID NO: 75 - DNA, 16 bp, Homo sapiens
atgcggtctatgtggt

SEQ ID NO: 76 - DNA, 16 bp, Homo sapiens
acggtgtcaccagcct

SEQ ID NO: 77 - DNA, 16 bp, Homo sapiens
aagctgtcccactctg

SEQ ID NO: 78 - DNA, 16 bp, Homo sapiens
gtgcggtcccatgatg

SEQ ID NO: 79 - DNA, 16 bp, Homo sapiens
gatgggtccgtgggca

SEQ ID NO: 80 - DNA, 16 bp, Homo sapiens
caagtgtcagacctgc

SEQ ID NO: 81 - DNA, 16 bp, Homo sapiens
agaccgtctgcgctcc

SEQ ID NO: 82 - DNA, 16 bp, Homo sapiens
tggcagtccccaaaaa

SEQ ID NO: 83 - DNA, 16 bp, Homo sapiens
aacatgtctctcctcg

SEQ ID NO: 84 - DNA, 16 bp, Homo sapiens
tttgcgtcatctttag

SEQ ID NO: 85 - DNA, 16 bp, Homo sapiens
gctgtgtccccacgga

SEQ ID NO: 86 - DNA, 16 bp, Homo sapiens
caatggtctttagaaa

SEQ ID NO: 87 - DNA, 16 bp, Homo sapiens
aaccagtcccagtttc

SEQ ID NO: 88 - DNA, 16 bp, Homo sapiens
ttttggtcccctccca

SEQ ID NO: 89 - DNA, 16 bp, Homo sapiens
agttcgtcatcctccc

SEQ ID NO: 90 - DNA, 16 bp, Homo sapiens
ctagggtctccaccta

SEQ ID NO: 91 - DNA, 16 bp, Homo sapiens
tgagtgtcctccaaga

SEQ ID NO: 92 - DNA, 16 bp, Homo sapiens
ccgcagtcacacatta

SEQ ID NO: 93 - DNA, 16 bp, Homo sapiens
tgccagtctggtcagg

SEQ ID NO: 94 - DNA, 16 bp, Homo sapiens
gtctggtcagggaagt

SEQ ID NO: 95 - DNA, 16 bp, Homo sapiens
gttgtgtcacatgtga

SEQ ID NO: 96 - DNA, 16 bp, Artificial
  Ribozyme substrate binding sequence
  misc_feature (1)..(16): n = T when DNA and U when RNA
nccnccccagaacccn

SEQ ID NO: 97 - DNA, 16 bp, Artificial
  Ribozyme substrate binding sequence
  misc_feature (1)..(16): n = T when DNA and U when RNA
nccagacaagaaagcn

SEQ ID NO: 98 - DNA, 16 bp, Homo sapiens
  misc_feature (7)..(7): n = T when DNA and U when RNA
agggagncggggagga

SEQ ID NO: 99 - DNA, 16 bp, Homo sapiens
  misc_feature (1)..(16): n = T when DNA and U when RNA
agcnggncngncngga

SEQ ID NO: 100 - DNA, 16 bp, Artificial
  Ribozyme substrate binding sequence
ctgacaaaagaagtct

SEQ ID NO: 101 - DNA, 16 bp, Artificial
  Ribozyme substrate binding sequence
gtaattctagaaaaga

SEQ ID NO: 102 - DNA, 16 bp, Artificial
  Ribozyme substrate binding sequence
tgtattgaagaagaaa

SEQ ID NO: 103 - DNA, 16 bp, Artificial
  Ribozyme substrate binding sequence
ccacataaagaaggaa

SEQ ID NO: 104 - DNA, 16 bp, Artificial
  Ribozyme substrate binding sequence
caagcccaagaaaaaa

SEQ ID NO: 105 - DNA, 16 bp, Artificial
  Ribozyme substrate binding sequence
cactgctaagaaagcc

SEQ ID NO: 106 - DNA, 16 bp, Artificial
  Ribozyme substrate binding sequence
aggcaacaagaaaaat

SEQ ID NO: 107 - DNA, 16 bp, Artificial
  Ribozyme substrate binding sequence
caacctgtagaaagag

SEQ ID NO: 108 - DNA, 16 bp, Artificial
  Ribozyme substrate binding sequence
agtccaatagaagcca

SEQ ID NO: 109 - DNA, 16 bp, Artificial
  Ribozyme substrate binding sequence
tttcctgaagaacttg

SEQ ID NO: 110 - DNA, 16 bp, Artificial
  Ribozyme substrate binding sequence
cctcaagaagaacacc

SEQ ID NO: 111 - DNA, 16 bp, Artificial
  Ribozyme substrate binding sequence
ttagtagaagaagggt

SEQ ID NO: 112 - DNA, 16 bp, Artificial
  Ribozyme substrate binding sequence
agtgcagtagaacgat

SEQ ID NO: 113 - DNA, 16 bp, Homo sapiens
agacagtctttgtcag

SEQ ID NO: 114 - DNA, 16 bp, Homo sapiens
tctttgtcagaattac

SEQ ID NO: 115 - DNA, 16 bp, Homo sapiens
tttctgtctcaataca

SEQ ID NO: 116 - DNA, 16 bp, Homo sapiens
ttcctgtcttatgtgg

SEQ ID NO: 117 - DNA, 16 bp, Homo sapiens
tttttgtctgggcttg

SEQ ID NO: 118 - DNA, 16 bp, Homo sapiens
ggcttgtctagcagtg

SEQ ID NO: 119 - DNA, 16 bp, Homo sapiens
atttagtctgttgcct

SEQ ID NO: 120 - DNA, 19 bp, Homo sapiens
ctcttgtcacaggttgrst

SEQ ID NO: 121 - DNA, 16 bp, Homo sapiens
tggctgtcattggact

SEQ ID NO: 122 - DNA, 16 bp, Homo sapiens
caagagtctcaggaaa

SEQ ID NO: 123 - DNA, 16 bp, Homo sapiens
ggtgggtctcttgagg

SEQ ID NO: 124 - DNA, 16 bp, Homo sapiens
accctgtctctactaa

SEQ ID NO: 125 - DNA, 16 bp, Homo sapiens
atcgtgtcactgcact

SEQ ID NO: 126 - DNA, 16 bp, Artificial
  Ribozyme substrate binding sequence
  misc_feature (1)..(16): n= T when DNA and U when RNA
anaccccnagaacnga

SEQ ID NO: 127 - DNA, 16 bp, Artificial
  Ribozyme substrate binding sequence
  misc_feature (1)..(16): n = T when DNA and U when RNA
acangnagagaacgca

SEQ ID NO: 128 - DNA, 16 bp, Artificial
  Ribozyme substrate binding sequence
  misc_feature (3)..(4): n = T when DNA and U when RNA
ggnnggcgagaaaagc

SEQ ID NO: 129 - DNA, 16 bp, Artificial
  Ribozyme substrate binding sequence
  misc_feature (1)..(16): n = T when DNA and U when RNA
ccacangnagaacgna

SEQ ID NO: 130 - DNA, 16 bp, Artificial
  Ribozyme substrate binding sequence
  misc_feature (1)..(16): n = T when DNA and U when RNA
gggnncgnagaacagg

SEQ ID NO: 131 - DNA, 16 bp, Artificial
  Ribozyme substrate binding sequence
  misc_feature (1)..(16): n = T when DNA and U when RNA
ncacngngagaaaagc

SEQ ID NO: 132 - DNA, 16 bp, Artificial
  Ribozyme substrate binding sequence
  misc_feature (1)..(16): n = T when DNA and U when RNA
aagngnggagaagngg

SEQ ID NO: 133 - DNA, 16 bp, Artificial
  Ribozyme substrtate binding sequence
agggaaaaagaaaagg

SEQ ID NO: 134 - DNA, 16 bp, Artificial
  Ribozyme substrate binding sequence
  misc_feature (1)..(16): n = T when DNA and U when RNA
annncacaagaagcca

SEQ ID NO: 135 - DNA, 16 bp, Artificial
  Ribozyme substrate binding sequence
  misc_feature (1)..(16): n = T when DNA and U when RNA
cagaanngagaacacc

SEQ ID NO: 136 - DNA, 16 bp, Artificial
  Ribozyme substrate binding sequence
  misc_feature (1)..(16): n = T when DNA and U when RNA
ncnngcngagaangag

SEQ ID NO: 137 - DNA, 16 bp, Artificial
  Ribozyme substrate binding sequence
  misc_feature (1)..(16): n = T when DNA and U when RNA
aaannngaagaancnc

SEQ ID NO: 138 - DNA, 16 bp, Artificial
  Ribozyme substrate binding sequence
  misc_feature (1)..(16): n = T when DNA and U when RNA
nnagannnagaaacnn

SEQ ID NO: 139 - DNA, 16 bp, Artificial
  Ribozyme substrate binding sequence
  misc_feature (1)..(16): n = T when DNA and U when RNA
nccagnnnagaanngg

SEQ ID NO: 140 - DNA, 16 bp, Homo sapiens
tggcagtctgtgaaat

SEQ ID NO: 141 - DNA, 16 bp, Homo sapiens
aagtcgtcaaatctaa

SEQ ID NO: 142 - DNA, 25 bp, Artificial
  Taqman probe
aatgcacgcaataaccggctcacat

SEQ ID NO: 143 - DNA, 25 bp, Artificial
  Primer
cacatccctcattatagtcagaaag

SEQ ID NO: 144 - DNA, 20 bp, Artificial
  Taqman primer
gagcagcatgtgggaaaaca

SEQ ID NO: 145 - DNA, 19 bp, Artificial
  Primer
gcatcacacacacagtgac

SEQ ID NO: 146 - DNA, 1731 bp, Artificial
  Contig
attttttgaggatttactattgccagacactgtgctacaagctgggaatgctgacagtataagataaagagggaaatgatgggggatgggtcatgtaaagggagaactttcatttttacttcatatatatctaagcagttataataggttgattttgtaatttaaaaaatgtaaaaatgcatacatgcactagtgcatgaatggcagccaggatgagtggaattggagaagcatcacacacacagtgacttttgtgtttgatcttgaagaatgagcgaaccaggcagggagtcggggaggagaatcgcattccttgaggaaagagcagcatgtgggaaaacataaatgcacgcaataacctggctcacatgttaagagaactttctgactataatgagggatgtgttgctgccccaagcttcattatctaaggagtttgttgaacactctctagaggcttttaataataggattgtttagctggtctgtctggactggttagatataacactatttaaatgacccaatctcattacattgtgaagatttccattttttaggttacgtaagaaattttggacctaaaaatcttgcattttaagacagtctttgtcagaattactttttggctctaaatgaattctgtaacatttgtattctaaattgacctttagtaaaagcaggaatggccatattcaaactgttacctcgcaaatcctgcccaccctttcactttctgtctcaatacattgatgtcctctaacccatttcctgtcttatgtggctttagtgccacttatcaaaattgtgtgcaaatttccttggctaacagtaacagtttttgtctgggcttgtctagcagtggaattctgcctgagttcatcatttttgtgactggtacttgaagtgcatcagatgattaatttcatgatcaacgtaccgggctcagtatgtgtcctgggtggtgatatagacatttatggaaaatgggatacccacattaagcagggtgactacctgtttaccatacaacccacacaaagccaatacaactatagatgtgctttatttagtctgttgcctctgcaaacattgcccgtgtgtttctctatgcccttcaaaaacatcagagcagcacatcctggaagatcctatcttttgtaagtttaagaagcagcctcttgtcacaggttgactcctaggtagtgtgcctagtgaccaagagggctgctaagaaagctttctgaccacttgtggctgtcattggactgatttgcccagatgacatcaattgggaatttgaggcatgacctataaagatcagttgcttgcaagagtctcaggaaaataattgtggagttaagaaacttgaagcgatttttaaaaattacctaacccaaccttctcatttgaaaaattaaaaaataaataggccagatatggcagctcatgcctgtaatcccagcgctgtgggagcctgaggcgggtgggtctcttgaggccaggagttcaagaccagcctggacaacatggtgaaaccctgtctctactaaaaacacaaaaattagctgagtgtggtggcaggcgcctgtaatcccagctactcaggaggctaaggtgggaggattacttgaaccggggaggcataggttgtagtgagccaagatcgtgtcactgcactccagcctgggtgacagagtaagactctgcctaaaaaaaaaaaaaaaaaaa

SEQ ID NO: 147 - DNA, 650 bp, Homo sapiens
  ref <308>: DATABASE ACCESSION NUMBER: GenBank BE877775
  ref <309>: DATABASE ENTRY DATE: 2000-09-26
aatggtgttagaatatattcctttctaatctgttttctatgcatgcacaaacacatatgtgagcacatatttataattttattctaaaaaataggatactgctgtacatattgttttacaatctaagtcatataattataatattctttaagcatatttatgagtaaaatattaaaacctatacaaaaaaataatagaatggcattttagctcattcattgatttttataaaactatttaacacactccctggtttgtacgttagcgttcaactaaatgctaaaaatttatcttcaccatcatcattaatttatttattaatcattattaaattcattcatctgatcattttcttgacggactttactattgccagacactgtgctacaagctgggaatgctgacagctataagcataaacgagggaaatgatggcgggatgggtcactgtacaacgggagaactttcatttttacttcatatatatctaagcagtcataataggttgatttcttgtaattctaaaaaatgtaaaaatgcatacatgcactagtgcatgaatggcagccaggatgagtggaatcggagcaagcatcacacacacagcgacttccgtgtccgatcttgaacgaatgagcgaaccaggccgg

SEQ ID NO: 148 - DNA, 2076 bp, Artificial
  Contig
aatggtgttagaatatattcctttctaatctgttttctatgcatgcacaaacacatatgtgagcacatatttataattttattctaaaaaataggatactgctgtacatattgttttacaatctaagtcatataattataatattctttaagcatatttatgagtaaaatattaaaacctatacaaaaaaataatagaatggcattttagctcattcattgatttttataaaactatttaacacactccctggtttgtacgttagcgttcaactaaatgctaaaaatttatcttcaccatcatcattaatttatttattaatcattattaaattcattcatctgatcattttcttgacggactttactattgccagacactgtgctacaagctgggaatgctgacagctataagcataaacgagggaaatgatggcgggatgggtcactgtacaacgggagaactttcatttttacttcatatatatctaagcagttataataggttgattttgtaatttaaaaaatgtaaaaatgcatacatgcactagtgcatgaatggcagccaggatgagtggaattggagaagcatcacacacacagtgacttttgtgtttgatcttgaagaatgagcgaaccaggcagggagtcggggaggagaatcgcattccttgaggaaagagcagcatgtgggaaaacataaatgcacgcaataacctggctcacatgttaagagaactttctgactataatgagggatgtgttgctgccccaagcttcattatctaaggagtttgttgaacactctctagaggcttttaataataggattgtttagctggtctgtctggactggttagatataacactatttaaatgacccaatctcattacattgtgaagatttccattttttaggttacgtaagaaattttggacctaaaaatcttgcattttaagacagtctttgtcagaattactttttggctctaaatgaattctgtaacatttgtattctaaattgacctttagtaaaagcaggaatggccatattcaaactggtaacctcgcaaatcctgcccaccctttcactttctgtctcaatacattgatgtcctctaacccatttcctgtcttatgtggctttagtgccacttatcaaaattgtgtgcaaatttccttggctaacagtaacagtttttgtctgggcttgtctagcagtggaattctgcctgagttcatcatttttgtgactggtacttgaagtgcatcagatgattaatttcatgataagagggctttttggggtggtgaaatagacatttatggaaaatgggatacccacattaagcagggtgactacctgtttaccatacaacccacacaaagccaatacaactatagatgtgctttatttagtctgttgcctctgcaaacattgcccgtgtgtttctctatgcccttcaaaaacatcagagcagcacatcctggaagatcctatcttttgtaagtttaagaagcagcctcttgtcacaggttgactcctaggtagtgtgcctagtgaccaagagggctgctaagaaagctttctgaccacttgtggctgtcattggactgatttgcccagatgacatcaattgggaatttgaggcatgacctataaagatcagttgcttgcaagagtctcaggaaaataattgtggagttaagaaacttgaagcgatttttaaaaattacctaacccaaccttctcatttgaaaaattaaaaaataaataggccagatatggcagctcatgcctgtaatcccagcgctgtgggagcctgaggcgggtgggtctcttgaggccaggagttcaagaccagcctggacaacatggtgaaaccctgtctctactaaaaacacaaaaattagctgagtgtggtggcaggcgcctgtaatcccagctactcaggaggctaaggtgggaggattacttgaaccggggaggcataggttgtagtgagccaagatcgtgtcactgcactccagcctgggtgacagagtaagactctgcctaaaaaaaaaaaaaaaaaaa

SEQ ID NO: 149 - DNA, 25 bp, Artificial
  Oligonucleotide primer
cacatccctcattatagtcagaaag

SEQ ID NO: 150 - DNA, 2377 bp, Artificial
  Contig
taaattgaatttttcctgcactggaatgatttgtttaattcttcttcgaacactgccctttctccagtaagaacactaatgatttgctaatattttttaaagaaatctgtttttttaattagttaagctcagacttcctcttattttttatcctagagaaaactgctaaaagggaatgatatatcagtactattcttctaaaacaactttttaaaaatgattatacaaagccaaatatgctcattatataaaatttagaagcaaaaagaaggaaataaaaattttccataattctaccagctagagataatggtgttagaatatattcctttctaatctgttttctatgcatgcacaaacacatatgtgagcacatatttataattttattctaaaaaataggatactgctgtacatattgttttacaatctaagtcatataattataatattctttaaacatatttatgagtaaaatattaaaacctatacaaaaaaataacagaatggcatttttagctcattcatggattttttataaaactatttaacacactcccctgggtttggtagttagcgttcaataaatgctaaaaatttattcttcaccatcatcattaatttatttattaatcattattaaattattcattgatcattttttgaggatttactattgccagacactgtgctacaagctgggaatgctgacagtataagataaagagggaaatgatgggggatgggtcatgtaaagggagaactttcatttttacttcatatatatctaagcagttataataggttgatttttgtaatttaaaaaatgtaaaaatgcatacatgcactagtgcatgaatggcagccaggatgagtggaattggagaagcatcacacacacagtgacttttgtgtttgatcttgaagaatgagcgaaccaggcagggagtcggggaggagaattgcattccttgaggaaagagcagcatgtgggaaaacataaatgcacgcaataacctggctcacatgttaagagaactttctgactataatgagggatgtgttgctgccccaagcttcattatctaaggagtttgttgaacactctctagaggcttttaataataggattgtttagctggtctgtctggactggttagatataacactatttaaatgacccaatctcattacattgtgaagatttccattttttaggttacgtaagaaattttggacctaaaaatcttgcattttaagacagtctttgtcagaattactttttggctctaaatgaattctgtaacatttgtattctaaattgacctttagtaaaagcaggaatggccatattcaaactggtaacctcgcaaatcctgcccaccctttcactttctgtctcaatacattgatgtcctctaacccatttcctgtcttatgtggctttagtgccacttatcaaaattgtgtgcaaatttccttggctaacagtaacagtttttgtctgggcttgtctagcagtggaattctgcctgagttcatcatttttgtgactggtacttgaagtgcatcagatgattaatttcatgataagagggctttttggggtggtgaaatagacatttatggaaaatgggatacccacattaagcagggtgactacctgtttaccatacaacccacacaaagccaatacaactatagatgtgctttatttagtctgttgcctctgcaaacattgcccgtgtgtttctctatgcccttcaaaaacatcagagcagcacatcctggaagatcctatcttttgtaagtttaagaagcagcctcttgtcacaggttgactcctaggtagtgtgcctagtgaccaagagggctgctaagaaagctttctgaccacttgtggctgtcattggactgatttgcccagatgacatcaattgggaatttgaggcatgacctataaagatcagttgcttgcaagagtctcaggaaaataattgtggagttaagaaacttgaagcgatttttaaaaattacctaacccaaccttctcatttgaaaaattaaaaaataaataggccagatatggcagctcatgcctgtaatcccagcgctgtgggagcctgaggcgggtgggtctcttgaggccaggagttcaagaccagcctggacaacatggtgaaaccctgtctctactaaaaacacaaaaattagctgagtgtggtggcaggcgcctgtaatcccagctactcaggaggctaaggtgggaggattacttgaaccggggaggcataggttgtagtgagccaagatcgtgtcactgcactccagcctgggtgacagagtaagactctgcctaaaaaaaaaaaaaaaaaaa

SEQ ID NO: 151 - DNA, 451 bp, Homo sapiens
  ref <308>: DATABASE ACCESSION NUMBER: GeBank BE327693
  ref <309>: DATABASE ENTRY DATE: 2000-07-14
aagtattgttaacaatcctttggaagtcactactggtctttgtgtgctgctttttaataattgagttattttgagcttgccaagtaggatctattgcctggactaaaatttatttcctaatcttctgatgaccaagaaaggaaaaattaagtttgcagatgtgagatgaaatatagccagtgaatatgcatactgattctgaatgaaaggaattaacttttcagtcaagaaacagtctgcatgcagtaaattgaatttcctgcaactggaatgatttgtttaattcttctttgaacactgccctttctccagtaagaacactaatgatttgctaatattttttaaagaaatctgtttttttaattagttaagctcacacttcctcttattttttatcctagagaaaactgctaaaagggaatgatatatcagtactattcttctaaaacaa

SEQ ID NO: 152 - DNA, 2624 bp, Artificial
  Contig
aagtattgttaacaatcctttggaagtcactactggtctttgtgtgctgctttttaataattgagttattttgagcttgccaagtaggatctattgcctggactaaaatttatttcctaatcttctgatgaccaagaaaggaaaaattaagtttgcagatgtgagatgaaatatagccagtgaatatgcatactgattctgaatgaaaggaattaacttttcagtcaagaaacagtctgcatgcagtaaattgaatttttcctgcaactggaatgatttgtttaattcttcttcgaacactgccctttctccagtaagaacactaatgatttgctaatattttttaaagaaatctgtttttttaattagttaagctcacacttcctcttattttttatcctagagaaaactgctaaaagggaatgatatatcagtactattcttctaaaacaactttttaaaaatgattatacaaagccaaatatgctcattatataaaatttagaagcaaaaagaaggaaataaaaattttccataattctaccagctagagataatggtgttagaatatattcctttctaatctgttttctatgcatgcacaaacacatatgtgagcacatatttataattttattctaaaaaataggatactgctgtacatattgttttacaatctaagtcatataattataatattctttaaacatatttatgagtaaaatattaaaacctatacaaaaaaataacagaatggcatttttagctcattcatggattttttataaaactatttaacacactcccctgggtttggtagttagcgttcaataaatgctaaaaatttattcttcaccatcatcattaatttatttattaatcattattaaattattcattgatcattttttgaggatttactattgccagacactgtgctacaagctgggaatgctgacagtataagataaagagggaaatgatgggggatgggtcatgtaaagggagaactttcatttttacttcatatatatctaagcagttataataggttgatttttgtaatttaaaaaatgtaaaaatgcatacatgcactagtgcatgaatggcagccaggatgagtggaattggagaagcatcacacacacagtgacttttgtgtttgatcttgaagaatgagcgaaccaggcagggagtcggggaggagaattgcattccttgaggaaagagcagcatgtgggaaaacataaatgcacgcaataacctggctcacatgttaagagaactttctgactataatgagggatgtgttgctgccccaagcttcattatctaaggagtttgttgaacactctctagaggcttttaataataggattgtttagctggtctgtctggactggttagatataacactatttaaatgacccaatctcattacattgtgaagatttccattttttaggttacgtaagaaattttggacctaaaaatcttgcattttaagacagtctttgtcagaattactttttggctctaaatgaattctgtaacatttgtattctaaattgacctttagtaaaagcaggaatggccatattcaaactggtaacctcgcaaatcctgcccaccctttcactttctgtctcaatacattgatgtcctctaacccatttcctgtcttatgtggctttagtgccacttatcaaaattgtgtgcaaatttccttggctaacagtaacagtttttgtctgggcttgtctagcagtggaattctgcctgagttcatcatttttgtgactggtacttgaagtgcatcagatgattaatttcatgataagagggctttttggggtggtgaaatagacatttatggaaaatgggatacccacattaagcagggtgactacctgtttaccatacaacccacacaaagccaatacaactatagatgtgctttatttagtctgttgcctctgcaaacattgcccgtgtgtttctctatgcccttcaaaaacatcagagcagcacatcctggaagatcctatcttttgtaagtttaagaagcagcctcttgtcacaggttgactcctaggtagtgtgcctagtgaccaagagggctgctaagaaagctttctgaccacttgtggctgtcattggactgatttgcccagatgacatcaattgggaatttgaggcatgacctataaagatcagttgcttgcaagagtctcaggaaaataattgtggagttaagaaacttgaagcgatttttaaaaattacctaacccaaccttctcatttgaaaaattaaaaaataaataggccagatatggcagctcatgcctgtaatcccagcgctgtgggagcctgaggcgggtgggtctcttgaggccaggagttcaagaccagcctggacaacatggtgaaaccctgtctctactaaaaacacaaaaattagctgagtgtggtggcaggcgcctgtaatcccagctactcaggaggctaaggtgggaggattacttgaaccggggaggcataggttgtagtgagccaagatcgtgtcactgcactccagcctgggtgacagagtaagactctgcctaaaaaaaaaaaaaaaaaaa

SEQ ID NO: 153 - DNA, 27 bp, Artificial
  Oligonucleotide primer
taacaatcctttggaagtcactactgg

SEQ ID NO: 154 - DNA, 21 bp, Artificial
  Oligonecleotide primer
aagcccagcattgctaagagg

SEQ ID NO: 155 - DNA, 3303 bp, Artificial
  Contig
aagcccagcattgctacgaggagagccaaccagcaagagacagagcagttttatttcacagtaagggagtgctatggcttttaaagagcgtcagcatgcactgcagcactcaagggagatttggaactcagagtccttgttaagtgtctgaaggacaggcgttgaatatcttagatacgaatgtgggcatactcagagaccatccctacagttaaaggtgcaaacattaaaagttgtatatgtctaacagggatccgcccaagagaaaggatgctcccaagtatacaactaagaagattttcttttttaagaaatttttaactagctagtaggctttcactggaaagtttccttctcaggcacaggggatcctgaaaggggaacttcatcttttagttcttggagagtacatacaaatattcataataacacatattttgtttataaaaatctataatctcttctaggtgatatgatgacattattttataacttttattgttgggaaactattttttctaattattgctaaaacttaaaggatgggtaatatgcagcattactattttgcacataattccaaaacatcgtattttcttattcatgtatctctagtcttcttttagacagttggacccttttttctctttttttttttttttttttttttttaagtattgttaacaatcctttggaagtcactactggtctttgtgtgctgctttttaataattgagttattttgagcttgccaagtaggatctattgcctggactaaaatttatttcctaatcttctgatgaccaagaaaggaaaaattaagtttgcagatgtgagatgaaatatagccagtgaatatgcatactgattctgaatgaaaggaattaacttttcagtcaagaaacagtctgcatgcagtaaattgaatttttcctgcaactggaatgatttgtttaattcttctttgaacactgccctttctccagtaagaacactaatgatttgctaatattttttaaagaaatctgtttttttaattagttaagctcagacttcctcttattttttatcctagagaaaactgctaaaagggaatgatatatcagtactattcttctaaaacaactttttaaaaatgattatacaaagccaaatatgctcattatataaaatttagaagcaaaaagaaggaaataaaaattttccataattctaccagctagagataatggtgttagaatatattcctttctaatctgttttctatgcatgcacaaacacatatgtgagcacatatttataattttattctaaaaaataggatactgctgtacatattgttttacaatctaagtcatataattataatattctttaagcatatttatgagtaaaatattaaaacctatacaaaaaaataacagaatggcattttagctcattcattgatttttataaaatatttaacacactccctggtttggtagttagcgttcaataaatgctaaaaatttatcttcaccatcatcattaatttatttattaatcattattaaattattcattgatcattttttgaggatttactattgccagacactgtgctacaagctgggaatgctgacagtataagataaagagggaaatgatgggggatgggtcatgtaaagggagaactttcatttttacttcatatatatctaagcagttataataggttgatttttgtaatttaaaaaatgtaaaaatgcatacatgcactagtgcatgaatggcagccaggatgagtggaattggagaagcatcacacacacagtgacttttgtgtttgatcttgaagaatgagcgaaccaggcagggagtcggggaggagaattgcattccttgaggaaagagcagcatgtgggaaaacataaatgcacgcaataacctggctcacatgttaagagaactttctgactataatgagggatgtgttgctgccccaagcttcattatctaaggagtttgttgaacactctctagaggcttttaataataggattgtttagctggtctgtctggactggttagatataacactatttaaatgacccaatctcattacattgtgaagatttccattttttaggttacgtaagaaattttggacctaaaaatcttgcattttaagacagtctttgtcagaattactttttggctctaaatgaattctgtaacatttgtattctaaattgacctttagtaaaagcaggaatggccatattcaaactggtaacctcgcaaatcctgcccaccctttcactttctgtctcaatacattgatgtcctctaacccatttcctgtcttatgtggctttagtgccacttatcaaaattgtgtgcaaatttccttggctaacagtaacagtttttgtctgggcttgtctagcagtggaattctgcctgagttcatcatttttgtgactggtacttgaagtgcatcagatgattaatttcatgatcaacgtaccagcctcagtatgtgtcctgcgtggtgaaatagacatttatggaaaatgggatacccacattaagcagggtgactacctgtttaccatacaacccacacaaagccaatacaactatagatgtgctttatttagtctgttgcctctgcaaacattgcccgtgtgtttctctatgcccttcaaaaacatcagagcagcacatcctggaagatcctatcttttgtaagtttaagaagcagcctcttgtcacaggttgactcctaggtagtgtgcctagtgaccaagagggctgctaagaaagctttctgaccacttgtggctgtcattggactgatttgcccagatgacatcaattgggaatttgaggcatgacctataaagatcagttgcttgcaagagtctcaggaaaataattgtggagttaagaaacttgaagcgatttttaaaaattacctaacccaaccttctcatttgaaaaattaaaaaataaataggccagatatggcagctcatgcctgtaatcccagcgctgtgggagcctgaggcgggtgggtctcttgaggccaggagttcaagaccagcctggacaacatggtgaaaccctgtctctactaaaaacacaaaaattagctgagtgtggtggcaggcgcctgtaatcccagctactcaggaggctaaggtgggaggattacttgaaccggggaggcataggttgtagtgagccaagatcgtgtcactgcactccagcctgggtgacagagtaagactctgcctaaaaaaaaaaaaaaaaaaa

SEQ ID NO: 156 - DNA, 935 bp, Homo sapiens
agagtaaagaagtcggccagcagctccaagatgatttgatgaaggtcctgaacgagctctactcggtcatgaagacatatcacatgtacaatgccgacagcatcagtgctcagagcaaactaaaggaggcggagaagcaggaggagaagcaaattggtaaatcggtaaagcaggaggaccggcagaccccacgctcccctgactccacggccaacgttcgcattgaggagaaacatgtccggaggagctcagtgaagaagattgagaagatgaaggagaagcgccaagccaagtacacggagaataagctgaaggccatcaaagcccggaatgagtacttgctggctttggaggcaaccaatgcatctgtcttcaagtactacatccatgacctatctgaccttattgatcagtgttgtgacttaggctaccatgcaagtctgaaccgggctctacgcaccttcctctctgctgagttaaacctggaacagtcgaagcatgagggtctggatgccatcgagaatgcagtagaaaacctggatgccaccagtgacaagcagcgcctcatggagatgtacaacaacgtcttctgcccccctatgaagtttgagtttcagccccacatgggggatatggcttcccagctctgtgcccagcagcctgtccagagtgagctggtacagagatgccaacaactgcagtctcgcttatccactctaaagattgaaaacgaagaggtaaagaagacaatggaggccaccctgcaaaccatccaggacattgtgactgtcgaggacttcgatgtgtctgactgcttccagtacagcaactccatggagtccgtcaagtccacggtctctgaaaccttcatgagcaagcccagcattgctaagaggagagccaaccagcaagagacagagcagttttatttcaca

SEQ ID NO: 157 - DNA, 4171 bp, Artificial
  Contig
  CDS (3)..(962)
tgcagagtaaagaagtcggccagcagctccaagatgatttgatgaaggtcctgaacgagctctactcggtcatgaagacatatcacatgtacaatgccgacagcatcagtgctcagagcaaactaaaggaggcggagaagcaggaggagaagcaaattggtaaatcggtaaagcaggaggaccggcagaccccacgctcccctgactccacggccaacgttcgcattgaggagaaacatgtccggaggagctcagtgaagaagattgagaagatgaaggagaagcgccaagccaagtacacggagaataagctgaaggccatcaaagcccggaatgagtacttgctggctttggaggcaaccaatgcatctgtcttcaagtactacatccatgacctatctgaccttattgatcagtgttgtgacttaggctaccatgcaagtctgaaccgggctctacgcaccttcctctctgctgagttaaacctggaacagtcgaagcatgagggtctggatgccatcgagaatgcagtagaaaacctggatgccaccagtgacaagcagcgcctcatggagatgtacaacaacgtcttctgcccccctatgaagtttgagtttcagccccacatgggggatatggcttcccagctctgtgcccagcagcctgtccagagtgagctggtacagagatgccaacaactgcagtctcgcttatccactctaaagattgaaaacgaagaggtaaagaagacaatggaggccaccctgcaaaccatccaggacattgtgactgtcgaggacttcgatgtgtctgactgcttccagtacagcaactccatggagtccgtcaagtccacggtctctgaaaccttcatgagcaagcccagcattgctaagaggagagccaaccagcaagagacagagcagttttatttcacagtaagggagtgctatggcttttaaagagcgtcagcatgcactgcagcactcaagggagatttggaactcagagtccttgttaagtgtctgaaggacaggcgttgaatatcttagatacgaatgtgggcatactcagagaccatccctacagttaaaggtgcaaacattaaaagttgtatatgtctaacagggatccgcccaagagaaaggatgctcccaagtatacaactaagaagattttcttttttaagaaatttttaactagctagtaggctttcactggaaagtttccttctcaggcacaggggatcctgaaaggggaacttcatcttttagttcttggagagtacatacaaatattcataataacacatattttgtttataaaaatctataatctcttctaggtgatatgatgacattattttataacttttattgttgggaaactattttttctaattattgctaaaacttaaaggatgggtaatatgcagcattactattttgcacataattccaaaacatcgtattttcttattcatgtatctctagtcttcttttagacagttggacccttttttctctttttttttttttttttttttttttaagtattgttaacaatcctttggaagtcactactggtctttgtgtgctgctttttaataattgagttattttgagcttgccaagtaggatctattgcctggactaaaatttatttcctaatcttctgatgaccaagaaaggaaaaattaagtttgcagatgtgagatgaaatatagccagtgaatatgcatactgattctgaatgaaaggaattaacttttcagtcaagaaacagtctgcatgcggggtaaattgaatttttcctgcaactggaatgatttgtttaattcttctttgaacactgccctttctccagtaagaacactaatgatttgctaatattttttaaagaaatctgtttttttaattagttaagctcagacttcctcttattttttatcctagagaaaactgctaaaagggaatgatatatcagtactattcttctaaaacaactttttaaaaatgattatacaaagccaaatatgctcattatataaaatttagaagcaaaaagaaggaaataaaaattttccataattctaccagctagagataatggtgttagaatatattcctttctaatctgttttctatgcatgcacaaacacatatgtgagcacatatttataattttattctaaaaaataggatactgctgtacatattgttttacaatctaagtcatataattataatattctttaaacatatttatgagtaaaatattaaaacttatacaaaaaaataacagaatggcattttagctcattcattgatttttataaaatatttaacacactccctggtttggtagttagcgttcaataaatgctaaaaatttattcttcaccatcatcattaatttatttattaatcattattaaattattcattgatcattttttgaggatttactattgccagacactgtgctacaagctgggaatgctgacagtataagataaagagggaaatgatgggggatgggtcatgtaaagggagaactttcatttttacttcatatatatctaagcagttataataggttgatttttgtaatttaaaaaatgtaaaaatgcatacatgcactagtgcatgaatggcagccaggatgagtggaattggagaagcatcacacacacagtgacttttgtgtttgatcttgaagaatgagcgaaccaggcagggagtcggggaggagaattgcattccttgaggaaagagcagcatgtgggaaaacataaatgcacgcaataacctggctcacatgttaagagaactttctgactataatgagggatgtgttgctgccccaagcttcattatctaaggagtttgttgaacactctctagaggcttttaataataggattgtttagctggtctgtctggactggttagatataacactatttaaatgacccaatctcattacattgtgaagatttccattttttaggttacgtaagaaattttggacctaaaaatcttgcattttaagacagtctttgtcagaattactttttggctctaaatgaattctgtaacatttgtattctaaattgacctttagtaaaagcaggaatggccatattcaaactggtaacctcgcaaatcctgcccaccctttcactttctgtctcaatacattgatgtcctctaacccatttcctgtcttatgtggctttagtgccacttatcaaaattgtgtgcaaatttccttggctaacagtaacagtttttgtctgggcttgtctagcagtggaattctgcctgagttcatcatttttgtgactggtacttgaagtgcatcagatgattaatttcatgataagagggctttttggggtggtgaaatagacatttatggaaaatgggatacccacattaagcagggtgactacctgtttaccatacaacccacacaaagccaatacaactatagatgtgctttatttagtctgttgcctctgcaaacattgcccgtgtgtttctctatgcccttcaaaaacatcagagcagcacatcctggaagatcctatcttttgtaagtttaagaagcagcctcttgtcacaggttgactcctaggtagtgtgcctagtgaccaagagggctgctaagaaagctttctgaccacttgtggctgtcattggactgatttgcccagatgacatcaattgggaatttgaggcatgacctataaagatcagttgcttgcaagagtctcaggaaaataattgtggagttaagaaacttgaagcgatttttaaaaattacctaacccaaccttctcatttgaaaaattaaaaaataaataggccagatatggcagctcatgcctgtaatcccagcgctgtgggagcctgaggcgggtgggtctcttgaggccaggagttcaagaccagcctggacaacatggtgaaaccctgtctctactaaaaacacaaaaattagctgagtgtggtggcaggcgcctgtaatcccagctactcaggaggctaaggtgggaggattacttgaaccggggaggcataggttgtagtgagccaagatcgtgtcactgcactccagcctgggtgacagagtaagactctgcctaaaaaaaaaaaaaaaaaaa

SEQ ID NO: 158 - protein, 319 aa, Artificial
  Contig
QSKEVGQQLQDDLMKVLNELYSVMKTYHMYNADSISAQSKLKEAEKQEEKQIGKSVKQEDRQTPRSPDSTANVRIEEKHVRRSSVKKIEKMKEKRQAKYTENKLKAIKARNEYLLALEATNASVFKYYIHDLSDLIDQCCDLGYHASLNRALRTFLSAELNLEQSKHEGLDAIENAVENLDATSDKQRLMEMYNNVFCPPMKFEFQPHMGDMASQLCAQQPVQSELVQRCQQLQSRLSTLKIENEEVKKTMEATLQTIQDIVTVEDFDVSDCFQYSNSMESVKSTVSETFMSKPSIAKRRANQQETEQFYFTVRECYGF

SEQ ID NO: 159 - DNA, 27 bp, Artificial
  Primer
cagagtaaagaagtcggccagcagctc

SEQ ID NO: 160 - DNA, 26 bp, Artificial
  Primer
gagaggtatgattggtggtaggatga

SEQ ID NO: 161 - DNA, 27 bp, Artificial
  Primer
cttcatcaaatcatcttggagctgctg

SEQ ID NO: 162 - DNA, 25 bp, Artificial
  Primer
gtaatgctgcatattacccatcctt

SEQ ID NO: 163 - DNA, 26 bp, Artificial
  Primer
tgcttctccgcctcctttagtttgct

SEQ ID NO: 164 - DNA, 6305 bp, Homo sapiens
  CDS (1)..(3288): Hypothetical protein
  ref <308>: DATABASE ACCESSION NUMBER: GenBank AB007925
  ref <309>: DATABASE ENTRY DATE: 1998-08-13
cggacgcgtgggttggattttttccgtggatctatcaattcacaattcgaatttggaagaaagaaggaaaacatgacgtctccagccaaattcaaaaaggataaggagatcatagcagagtacgatactcaggtcaaagagatccgtgctcagctcacagagcagatgaaatgcctggaccagcagtgtgagcttcgggtgcaactgttgcaggacctccaggacttcttccgaaagaaggcagagattgagatggactactcccgcaacctggagaagctggcagaacgcttcctggccaagacacgcagcaccaaggaccagcaattcaagaaggatcagaatgttctctctccagtcaactgctggaatctcctcttaaaccaggtgaagcgggaaagcagggaccataccaccctgagtgacatctacctgaataatatcattcctcgatttgtacaagtcagcgaggactcaggaagactctttaaaaagagtaaagaagtcggccagcagctccaagatgatttgatgaaggtcctgaacgagctctactcggtgatgaagacatatcacatgtacaatgccgacagcatcagtgctcagagcaaactaaaggaggcggagaagcaggaggagaagcaaattggtaaatcggtaaagcaggaggaccggcagaccccacgctcccctgactccacggccaacgttcgcattgaggagaaacatgtccggaggagctcagtgaagaagattgagaagatgaaggagaagcgtcaagccaagtacacggagaataagctgaaggccatcaaagcccggaatgagtacttgctggctttggaggcaaccaatgcatctgtcttcaagtactacatccatgacctatctgaccttattgatcagtgttgtgacttaggctaccatgcaagtctgaaccgggctctacgcaccttcctctctgctgagttaaacctggaacagtcgaagcatgagggtctggatgccatcgagaatgcagtagaaaacctggatgccaccagtgacaagcagcgcctcatggagatgtacaacaacgtcttctgcccccctatgaagtttgagtttcagccccacatgggggatatggcttcccagctctgtgcccagcagcctgtccagagtgagctggtacagagatgccaacaactgcagtctcgcttatccactctaaagattgaaaacgaagaggtaaagaagacaatggaggccaccctgcaaaccatccaggacattgtgactgtcgaggactttgatgtgtctgactgcttccagtacagcaactccatggagtccgtcaagtccacggtctctgaaaccttcatgagcaagcccagcattgctaagaggagagccaaccagcaagagacagagcagttttatttcacaaaaatgaaagagtacctggagggcaggaacctcatcaccaagttacaagccaagcatgaccttctgcagaaaaccctgggagaaagtcagcggacagattgcagtctagccaggcgcagctcaactgtgaggaaacaggactccagccaggcaattcctctggtggtggaaagctgtatccggtttatcagcagacacggactacagcatgaaggaattttccgggtgtcaggatcccaggtggaagtgaatgacatcaaaaatgcctttgagagaggagaggaccccctggctggggaccagaacgaccatgacatggattccatagctggtgtcctgaagctttacttccgggggctggaacaccctctcttccccaaggacatctttcatgacctgatggcctgcgtcacaatggacaacctgcaggagagagctctgcacatccggaaagtcctcctagtcctgcccaaaaccactctgattatcatgagatacctctttgccttcctcaatcatttatcacagttcagtgaagagaacatgatggacccctacaacctcgccatctgcttcgggccctcgctaatgtcagtgccagagggccacgaccaggtgtcctgccaagcccacgtgaatgagctgatcaaaaccatcatcatccagcatgagaacatcttcccaagccccagggagctggagggccctgtctacagcagaggaggaagcatggaggattactgtgatagccctcatggagagactaccccggttgaagactcaacccaggatgtgaccgcagagcaccacacgagcgatgacgaatgtgagcccatcgaggccattgccaagtttgactacgtgggccggacagcccgagagctgtcctttaagaagggagcatccctgctgctttaccagcgggcttccgacgactggtgggaaggccggcacaatggcatcgacggactcatcccccatcagtacatcgtggtccaagacaccgaggacggtgtcgtggagaggtccagccccaagtctgagattgaggtcatttctgagccacctgaagaaaaggtgacagccagagcgggggccagctgtcccagtgggggtcatgtagccgatatttatcttgcaaacatcaacaagcaaaggaagcgtccagaatctgggagcatccggaaaacttttcggagtgacagccatgggctgagcagttccctgactgactcctcctccccaggggtgggggctagctgccgcccatcctcccagcccatcatgagccagagcctccccaaagaagggccagataagtgttccatcagtgggcacgggagcctcaactccatcagccgccactcatccctgaagaatcggctggatagtccacagatccggaagactgccacagcgggaaggtcaaaaagcttcaataaccatcggcccatggaccctgaggtcattgctcaggatattgaggcaacaatgaactcggccctgaatgagctacgggaactagaacggcagagcagtgtcaaacacacccctgacgtggttctggacaccttggagcccctcaaaacctccccagtggtggcccccacgtcagagccctccagccctctgcacacccagctcctcaaggaccccgagcccgccttccagcgcagcgccagtactgctggggacatcgcctgcgccttccggcctgtcaagtctgtcaagatggctgccccggtcaaaccaccagccacacggcccaagcccactgtcttccccaaaacaaatgccactagccctggtgtcaactcatcaacttccccacagtctactgacaagtcttgtactgtctgagggataataatttaattgttctagacaaggggactatagggactgactgttattaaaatcttcctatttaactagcttggggacttcagttgaaaattaggttctaagttgttcttgcaggaattagcctccccgtctcccaaaaccttgagaatgaagcccttggtatcgcctctcccttcccactgccctctgcttcccccagtcgtcgtaattcagccagctgcagtccgtaccgttcttaggttagccagagacaggttttcattatcaggtcactgtgaaatctggtaaggcagtcctgaggacatgggctcaagtctcagtcccctcagaccacggtgatgccttgaccagatggttggctactgccatccagctttcagtggcatcttgttttgggaactgattatagagaatcatatatagtccagtcttcattttacatacacacacatattttacacacacacacattttacacacacacacacacacacacacacacacatatgtttttctagtctctggcatgtgtagcctctcctggtcatagaccagtctctttgtaagttattgtggcagttcacacagtagccaccaggggtctctgtttccattacaaactttgttctgtctgggccagagaacctagcctttgaaatctctccatcatgtgaaacataacgggatgggacaatcccgtaacctgtttggggttgggggctttctctctgtgttctttccattgatgtgaattggtcattggtgtttgctcttgcctctcctccatcccctagaagtacacccccgtcttattaaggagcttttaaagtttttctgaatgtatagacatttctcgggttcctacctttgtctctgatggaccattttccatttaagacattttcctgtataagacagttttatagctggttccttttagggtaaagagtcttaagagagttttattgtgtctatggcaggtttgggaaaggtaagaaatgggtcctttttcctcctaatgtttttggcacttaaaacataaaattcattatcctattaaaaaattaaattcagctttgctaatccagaaattgttcccaaatgaaaacttgttttaagtccaccccttagtttccttattttacaaggtctctcttcagggaccaacaggggcttagagagccttagttagattaaagggagaccctacctcttaaaaccagttttcatttatgcaaacaaggacaattaagggaaccctgaccccacaggctctcaagtcttcccaaggccagaatcgaaagaaaattaaaatttgaatgctgaatattctggctctactctggccttttttctggttcccttccaaaatgcacaaatcatacccttgtctgctccaattcagtctccaaacctggtgcctgtgctcctggcccccctagcatcatgctatcccaggagtatcaggaccagacacatccacagccaggctcatgggtctcagacagcaacttgagttaaagctgaaactcatccttcttctctgtgttttctggtttaaaagctgcacttatattttagccttattattttctgtagttccggagagatggtgggttgccattctggtaggaaaatctgagttttcttatctttgacctagaagagattctttttggaccaacctgcgaaattggtagttaggtctatggaaagtggtaggatttttttttttttaatcctgtgcaaaggaaaagaggtgctttgtgggaaatcactaatgagaaggctaacctgcagcaccagagaaacctttccaagtgctaggcaggagaactgaagaactctttcagtgaagtgagtcagcctagaagaggcaacccacagtcttgatttttgttgctgtttcctgacctgttcttgcctgtcacctgggcctacacaggtcccgagccaacggggctttcatacccaaggatctgtttccttgctgaaaatgaaaccctatctttcactttacattcctttcaatccaactgatcaaaactggtacccacacttgccctttctccctctctccagcacactcccctctaagaaagtaaaagcaaagctttcttaatggcaacactttgggcctgttctgttgctcctgccttatttctctttcaaccctgtacatgactcgtgttcaccatccccttgatcagtgtccatccgcgctaatttgcatcatgaactgaacagtgtgtgagtggtcacctactaaacccagctctgggggcagagctgtcttccccatctctggtgctcctaacacctgtgagatggtcctgtcgagaggactaggaaccgataggaggagagtccttctcggcagagctcactgcaaacaactggaattgaggttgcacactgtgatttttacaccgaaaagccaaaaggagctggccatccagggcctagggagaccagccttcctcagctatgcttgccgaaaccagcatgatgcttcaaagagccctgctccacccctcatggcatggatccttttcctggtgtggactctgaagggtcagtcttcggggaagagaggtggggtggggctactagcatcccaatttagaaaatagaggagtttgtagccagcagcctgtaaactggaaacactggtctcagccaacctcctcagggcgccctggcttctccccaaggagatgaggagcggtgatgccagcaccgggatgcgcagagcactggaagggctggtgcagatctacttcccatgcagaagagaagtcacatcttccagggaatcgcaatgttgtggcgtctgacttgtatgtcacatttgtgtaaaatggtatattctttaaaatagtgttgataactggaatattgtatgtatgcttggagatgctttgtgtgaacctaagactgtcactcaacagatgttggattggggaaaatccaaagcacaacttcaaaataaaatacatttttaggtttcg

SEQ ID NO: 165 - protein, 1095 aa, Homo sapiens
RTRGLDFFRGSINSQFEFGRKKENMTSPAKFKKDKEIIAEYDTQVKEIRAQLTEQMKCLDQQCELRVQLLQDLQDFFRKKAEIEMDYSRNLEKLAERFLAKTRSTKDQQFKKDQNVLSPVNCWNLLLNQVKRESRDHTTLSDIYLNNIIPRFVQVSEDSGRLFKKSKEVGQQLQDDLMKVLNELYSVMKTYHMYNADSISAQSKLKEAEKQEEKQIGKSVKQEDRQTPRSPDSTANVRIEEKHVRRSSVKKIEKMKEKRQAKYTENKLKAIKARNEYLLALEATNASVFKYYIHDLSDLIDQCCDLGYHASLNRALRTFLSAELNLEQSKHEGLDAIENAVENLDATSDKQRLMEMYNNVFCPPMKFEFQPHMGDMASQLCAQQPVQSELVQRCQQLQSRLSTLKIENEEVKKTMEATLQTIQDIVTVEDFDVSDCFQYSNSMESVKSTVSETFMSKPSIAKRRANQQETEQFYFTKMKEYLEGRNLITKLQAKHDLLQKTLGESQRTDCSLARRSSTVRKQDSSQAIPLVVESCIRFISRHGLQHEGIFRVSGSQVEVNDIKNAFERGEDPLAGDQNDHDMDSIAGVLKLYFRGLEHPLFPKDIFHDLMACVTMDNLQERALHIRKVLLVLPKTTLIIMRYLFAFLNHLSQFSEENMMDPYNLAICFGPSLMSVPEGHDQVSCQAHVNELIKTIIIQHENIFPSPRELEGPVYSRGGSMEDYCDSPHGETTPVEDSTQDVTAEHHTSDDECEPIEAIAKFDYVGRTARELSFKKGASLLLYQRASDDWWEGRHNGIDGLIPHQYIVVQDTEDGVVERSSPKSEIEVISEPPEEKVTARAGASCPSGGHVADIYLANINKQRKRPESGSIRKTFRSDSHGLSSSLTDSSSPGVGASCRPSSQPIMSQSLPKEGPDKCSISGHGSLNSISRHSSLKNRLDSPQIRKTATAGRSKSFNNHRPMDPEVIAQDIEATMNSALNELRELERQSSVKHTPDVVLDTLEPLKTSPVVAPTSEPSSPLHTQLLKDPEPAFQRSASTAGDIACAFRPVKSVKMAAPVKPPATRPKPTVFPKTNATSPGVNSSTSPQSTDKSCTV

SEQ ID NO: 166 - DNA, 5428 bp, Artificial
  Contig
  CDS (1)..(999)
gaatgcctggaccagcagtgtgagcttcgggtgcaactgttgcaggacctccaggacttcttccgaaagaaggcagagattgagatggactactcccgcaacctggagaagctggcagaacacttcctggccaagacacgcagcaccaaggaccagcaattcaagaaggatcagaatgttctctctccagtcaactgctggaatctcctcttaaaccaggtgaagtgggaaagcagggaccataccaccctgagtgacatctacctgaataatatcattcctcgatttgtacaagtcagcgaggactcaggaagactctttaaaaagagtaaagaagtcggccagcagctccaagatgatttgatgaaggtcctgaacgagctctactcggtcatgaagacatatcacatgtacaatgccgacagcatcagtgctcagagcaaactaaaggaggcggagaagcaggaggagaagcaaattggtaaatcggtaaagcaggaggaccggcagaccccacgctcccctgactccacggccaacgttcgcattgaggagaaacatgtccggaggagctcagtgaagaagattgagaagatgaaggagaagcaccaagccaagtacacggagaataagctgaaggccatcaaagcccagaatgagtacttgctggctttggaggcaaccaatgcatctgtcttcaagtactacatccatgacctatctgaccttattgatcagtgttgtgacttaggctaccatgcaagtctgaaccgggctctacgcaccttcctctctgctgagttaaacctggaacagtcgaagcatgagggtctggatgccatcgagaatgcagtagaaaacctggatgccaccagtgacaagcagcgcctcatggagatgtacaacaacgtcttctgcccccctatgaagtttgaagttttcagccccacattgggggatatggccttcccagctctgtgcccagcagcctgtccagagtgagctggtacagagatgccaacaactgcagtctcgcttatccactctaaagattgaaaacgaagaggtaaagaagacaatggaggccaccctgcaaacccatccaggacattgtgactgtcgaggacttcgatgtgtctgactgcttccagtacagcaactccatggagtccgtcaagtccacggtctctgaaaccttcatgagcaagcccagcattgctacgaggagagccaaccagcaagagacagagcagttttatttcacagtaagggagtgctatggcttttaaagagcgtcagcatgcactgcagcactcaagggagatttggaactcagagtccttgttaagtgtctgaaggacaggcgttgaatatcttagatacgaatgtgggcatactcagagaccatccctacagttaaaggtgcaaacattaaaagttgtatatgtctaacagggatccgcccaagagaaaggatgctcccaagtatacaactaagaagattttcttttttaagaaatttttaactagctagtaggctttcactggaaagtttccttctcaggcacaggggatcctgaaaggggaacttcatcttttagttcttggagagtacatacaaatattcataataacacatattttgtttataaaaatctataatctcttctaggtgatatgatgacattattttataacttttattgttgggaaactattttttctaattattgctaaaacttaaaggatgggtaatatgcagcattactattttgcacataattccaaaacatcgtattttcttattcatgtatctctagtcttcttttagacagttggacccttttttctctttttttttttttttttttttttttaagtattgttaacaatcctttggaagtcactactggtctttgtgtgctgctttttaataattgagttattttgagcttgccaagtaggatctattgcctggactaaaatttatttcctaatcttctgatgaccaagaaaggaaaaattaagtttgcagatgtgagatgaaatatagccagtgaatatgcatactgattctgaatgaaaggaattaacttttcagtcaagaaacagtctgcatgcagtaaattgaatttttcctgcaactggaatgatttgtttaattcttctttgaacactgccctttctccagtaagaacactaatgatttgctaatattttttaaagaaatctgtttttttaattagttaagctcagacttcctcttattttttatcctagagaaaactgctaaaagggaatgatatatcagtactattcttctaaaacaactttttaaaaatgattatacaaagccaaatatgctcattatataaaatttagaagcaaaaagaaggaaataaaaattttccataattctaccagctagagataatggtgttagaatatattcctttctaatctgttttctatgcatgcacaaacacatatgtgagcacatatttataattttattctaaaaaataggatactgctgtacatattgttttacaatctaagtcatataattataatattctttaagcatatttatgagtaaaatattaaaacctatacaaaaaaataacagaatggcattttagctcattcattgatttttataaaatatttaacacactccctggtttggtagttagcgttcaataaatgctaaaaatttatcttcaccatcatcattaatttatttattaatcattattaaattattcattgatcattttttgaggatttactattgccagacactgtgctacaagctgggaatgctgacagtataagataaagagggaaatgatgggggatgggtcatgtaaagggagaactttcatttttacttcatatatatctaagcagttataataggttgatttttgtaatttaaaaaatgtaaaaatgcatacatgcactagtgcatgaatggcagccaggatgagtggaattggagaagcatcacacacacagtgacttttgtgtttgatcttgaagaatgagcgaaccaggcagggagtcggggaggagaattgcattccttgaggaaagagcagcatgtgggaaaacataaatgcacgcaataacctggctcacatgttaagagaactttctgactataatgagggatgtgttgctgccccaagcttcattatctaaggagtttgttgaacactctctagaggcttttaataataggattgtttagctggtctgtctggactggttagatataacactatttaaatgacccaatctcattacattgtgaagatttccattttttaggttacgtaagaaattttggacctaaaaatcttgcattttaagacagtctttgtcagaattactttttggctctaaatgaattctgtaacatttgtattctaaattgacctttagtaaaagcaggaatggccatattcaaactggtaacctcgcaaatcctgcccaccctttcactttctgtctcaatacattgatgtcctctaacccatttcctgtcttatgtggctttagtgccacttatcaaaattgtgtgcaaatttccttggctaacagtaacagtttttgtctgggcttgtctagcagtggaattctgcctgagttcatcatttttgtgactggtacttgaagtgcatcagatgattaatttcatgatcaacgtaccagcctcagtatgtgtcctgcgtggtgaaatagacatttatggaaaatgggatacccacattaagcagggtgactacctgtttaccatacaacccacacaaagccaatacaactatagatgtgctttatttagtctgttgcctctgcaaacattgcccgtgtgtttctctatgcccttcaaaaacatcagagcagcacatcctggaagatcctatcttttgtaagtttaagaagcagcctcttgtcacaggttgactcctaggtagtgtgcctagtgaccaagagggctgctaagaaagctttctgaccacttgtggctgtcattggactgatttgcccagatgacatcaattgggaatttgaggcatgacctataaagatcagttgcttgcaagagtctcaggaaaataattgtggagttaagaaacttgaagcgatttttaaaaattacctaacccaaccttctcatttgaaaaattaaaaaataaataggccagatatggcagctcatgcctgtaatcccagcgctgtgggagcctgaggcgggtgggtctcttgaggccaggagttcaagaccagcctggacaacatggtgaaaccctgtctctactaaaaacacaaaaattagctgagtgtggtggcaggcgcctgtaatcccagctactcaggaggctaaggtgggaggattacttgaaccggggaggcataggttgtagtgagccaagatcgtgtcactgcactccagcctgggtgacagagtaagactctgcctaaaaaaaagaaagattaaaaaataaataaatctgctgggcgtggtggctcacgcctgtaatcccagcactttgggaggccgaggcgggcggatcacctgaggtcgggagtttgagaccagcctgatcaacatggagaaacctcgtctctactaaaaacacgaaaagattagctgggcgtggtggcgcatgcctcattcacgtacatgggagagtctacaaagtcacacgtattcataggttaagccacatgctgacaaatgtcataagaagaccctacacttttaccttggccgatccctcccctcagtgcaagctctgtgcaagagtgaacttgaacttcactcagtgcaagagtgaacacacactttgtgccggctttaaagaacccagcacaaagccagtctgcatggcctacagacatattttgctggacaatgattacttgcttttctttttgtttttcttgtatttgcccgtttgattgggctcctgacatacccagaaaatcactgtccaaaacattagcttaacatttgttaaggaaacaaaaagacttcggtgaccacaccttataaaccaaacagttttgtaaatcactttggaaaatttcactaaaaaaaaaaatccttaacaatataataagtaaagaaaatttaaaaccacaaaacatcactgtgtttgtagggggaggtctgatttacagagtaaccacatagtaattataattattagaatgtccagttttcaaaaaacgttacaaggcatacaaagaatgggaaagtgtggctcattcaaaggaacaaaataaagtgacagaaaatatccctaaagaaacccagacatcaaacttaccagacaaagactttaaaacaactgtcttcattatactcaaatgtcaaaaggaaaacataaaaaaaaaaaaaaaaaaaaaaaaaaaa

SEQ ID NO: 167 - protein, 333 aa, Artificial
  Contig
ECLDQQCELRVQLLQDLQDFFRKKAEIEMDYSRNLEKLAEHFLAKTRSTKDQQFKKDQNVLSPVNCWNLLLNQVKWESRDHTTLSDIYLNNIIPRFVQVSEDSGRLFKKSKEVGQQLQDDLMKVLNELYSVMKTYHMYNADSISAQSKLKEAEKQEEKQIGKSVKQEDRQTPRSPDSTANVRIEEKHVRRSSVKKIEKMKEKHQAKYTENKLKAIKAQNEYLLALEATNASVFKYYIHDLSDLIDQCCDLGYHASLNRALRTFLSAELNLEQSKHEGLDAIENAVENLDATSDKQRLMEMYNNVFCPPMKFEVFSPTLGDMAFPALCPAACPE

SEQ ID NO: 168 - DNA, 494 bp, Artificial
  Probe
atgcatgcacaaacacatatgtgagcacatatttataattttattctaaaaaataggatactgctgtacatattgttttacaatctaagtcatataattataatattctttaaacatatttatgagtaaaatattaaaacctatacaaaaaaataacagaatggcatttttagctcattcatggattttttataaaactatttaacacactcccctgggtttggtagttagcgttcaataaatgctaaaaatttattcttcaccatcatcattaatttatttattaatcattattaaattattcattgatcattttttgaggatttactattgccagacactgtgctacaagctgggaatgctgacagtataagataaagagggaaatgatgggggatgggtcatgtaaagggagaactttcatttttacttcatatatatctaagcagttataataggttgatttttgtaatttaaaaaatgtaaaaatgcat

SEQ ID NO: 169 - protein, 616 aa, Homo sapiens
MLHNLNQQRKNGGRFCDVLLRVGDESFPAHRAVLAACSEYFESVFSAQLGDGGAADGGPADVGGATAAPGGGAGGSRELEMHTISSKVFGDILDFAYTSRIVVRLESFPELMTAAKFLLMRSVIEICQEVIKQSNVQILVPPARADIMLFRPPGTSDLGFPLDMTNGAALAANSNGIAGSMQPEEEAARAAGAAIAGQASLPVLPGVDRLPMVAGPLSPQLLTSPFPSVASSAPPLTGKRGRGRPRKANLLDSMFGSPGGLREAGILPCGLCGKVFTDANRLRQHEAQHGVTSLQLGYIDLPPPRLGENGLPISEDPDGPRKRSRTRKQVACEICGKIFRDVYHLNRHKLSHSGEKPYSCPVCGLRFKRKDRMSYHVRSHDGSVGKPYICQSCGKGFSRPDHLNGHIKQVHTSERPHKCQTCNASFATRDRLRSHLACHEDKVPCQVCGKYLRAAYMADHLKKHSEGPSNFCSICNREGQKCSHQDPIESSDSYGDLSDASDLKTPEKQSANGSFSCDMAVPKNKMESDGEKKYPCPECGSFFRSKSYLNKHIQKVHVRALGGPLGDLGPALGSPFSPQQNMSLLESFGFQIVQSAFASSLVDPEVDQQPMGPEGK

SEQ ID NO: 170 - protein, 440 aa, Homo sapiens
MEGVLYKWTNYLSGWQPRWFLLCGGILSYYDSPEDAWKGCKGSIQMAVCEIQVHSVDNTRMDLIIPGEQYFYLKARSVAERQRWLVALGSAKACLTDSRTQKEKEFAENTENLKTKMSELRLYCDLLVQQVDKTKEVTTTGVSNSEEGIDVGTLLKSTCNTFLKTLEECMQIANAAFTSELLYRTPPGSPQLAMLKSSKMKHPIIPIHNSLERQMELSTCENGSLNMEINGEEEILMKNKNSLYLKSAEIDCSISSEENTDDNITVQGEIRKEDGMENLKNHDNNLTQSGSDSSCSPECLWEEGKEVIPTFFSTMNTSFSDIELLEDSGIPTEAFLASCYAVVPVLDKLGPTVFAPVKMDLVGNIKKVNQKYITNKEEFTTLQKIVLHEVEADVAQVRNSATEALLWLKRGLKFLKGFLTEVKNGEKDIQTALRNPTENT

SEQ ID NO: 171 - protein, 537 aa, Homo sapiens [sequence omitted]

(19) United States
(12) Patent Application Publication
(10) Pub. No.: US 2004/0248830 A1
(43) Pub. Date: Dec. 9, 2004

(54) Agents that regulate apoptosis

(76) Inventors: Richard Tritz, San Diego, CA (US); Benjamin Keily, San Diego, CA (US); Cellia Habita, San Diego, CA (US); Joan Robbins, San Diego, CA (US); Jack Barber, San Diego, CA (US)

Correspondence Address: Commissioner for Patents, PO Box 1450, Alexandria, VA, 22313-1450, US

(21) Appl. No.: 10/478,019

(22) PCT Filed: May 14, 2002
(86) PCT No.: PCT/US02/15198

Related U.S. Application Data
(60) Provisional application No. 60/290,927, filed on May 14, 2001.

(51) Int. Cl. A61K 48/00; C07H 21/04

(52) U.S. Cl. 514/44; 536/23.1

(57) ABSTRACT

The present invention provides genes, nucleic acid sequences, proteins and amino acid sequences that are, involved in the inhibition of apoptosis induction in cells. The invention also provides the RNA correlates of these genes and nucleic acid sequences. Further provided are isolated nucleic acid molecules that interact with the genes, nucleic acid sequences and RNA correlates disclosed herein, such that their inhibitory effect on apoptosis induction is lessened and, therefore, apoptosis induction is facilitated.

### FIELD OF THE INVENTION

The invention relates generally to the identification of agents involved in disease processes and more particularly to the identification of genes, nucleic acid sequences, proteins and amino acid sequences involved in the inhibition of apoptosis induction. The invention also relates to the identification of nucleic acids encoding ribozymes, the ribozymes themselves and their use for facilitating the induction of apoptosis.

### BACKGROUND OF THE INVENTION

Apoptosis or programmed cell death is an active process of gene-directed cellular self-destruction that contrasts fundamentally with degenerative death or necrosis. Apoptotic cell death is characterized by cellular shrinkage, chromatin condensation, cytoplasmic blebbing, increased membrane permeability and interchromosomal DNA cleavage.

The process of programmed cell death through apoptosis is connected with a variety of normal and pathogenic biological events and can be induced by a number of unrelated stimuli. Many biological processes, including embryogenesis, immune system responses, elimination of virus-infected cells, and the maintenance of tissue homeostasis involve apoptosis. Changes in the biological regulation of apoptosis also occur during aging and are responsible for many of the conditions and diseases related to aging.

Because of the role apoptosis plays in normal physiology, the failure of apoptosis to occur in such processes can contribute to disease. For example, substantial evidence has accumulated that inhibition of apoptotic processes is involved in the formation or growth of cancer. As a result, the elimination of cancer cells through induction of apoptotic cell death is now considered a promising approach in cancer therapy. A variety of chemotherapeutic compounds as well as ionizing radiation that have been used successfully in cancer treatments have been demonstrated to induce apoptosis in tumor cells, for example by activating well known apoptosis induction genes such as wild-type p53.

However, the process of apoptosis induction in cancer and in other disease states is complex and much more needs to be done to identify genes and gene products involved in regulating apoptosis. In this regard, the discovery of new agents that affect the activity of genes or gene products that inhibit apoptosis will have therapeutic utility in a wide variety of conditions. The present invention addresses these needs and provides related advantages as well.

### SUMMARY OF THE INVENTION

The present invention provides genes, nucleic acid sequences, proteins and amino acid sequences that are involved in the inhibition of apoptosis induction in cells. The invention also provides the RNA correlates of these genes and nucleic acid sequences. Further provided are isolated nucleic acid molecules that interact with the genes, nucleic acid sequences and RNA correlates disclosed herein, such that their inhibitory effect on apoptosis induction is lessened and, therefore, apoptosis induction is facilitated.

The isolated nucleic acid molecules of the invention include nucleotide sequences encoding ribozymes. The ribozymes of the invention have “substrate binding sequences” that hybridize to and cleave complementary sequences of the mRNA encoded by the genes and gene sequences disclosed herein and, therefore, facilitate apoptosis. Included within the scope of the invention are expression vectors encoding the ribozymes, cells containing the vectors and cells expressing the ribozymes. In addition, as shown for instance in Example 6, a ribozyme of the present invention can be introduced directly into a cell, i.e., without the use of a vector.

The present invention further provides a method for facilitating the induction of apoptosis in cells, for example cells resistant to apoptosis induction such as cancer cells, by introducing a ribozyme of the invention into such cells. For example, the cells can be transduced with expression vectors encoding ribozymes of the invention and, optionally, an apoptosis inducing agent can be introduced in the cells.

The present invention further provides a method for identifying an agent that can facilitate induction of apoptosis in cells, for example those resistant to apoptosis induction. The method comprises contacting a protein or polypeptide encoded by the genes and gene sequences disclosed herein, or contacting these genes or gene sequences themselves, with the agent and measuring the level or activity of the protein or polypeptide. A reduction in level would indicate that the agent can facilitate induction of apoptosis. Representative agents include antisense oligonucleotides, monoclonal and polyclonal antibodies, and small molecule drugs.

### BRIEF DESCRIPTION OF THE DRAWINGS

FIG. 1 shows the general structure and nucleotide sequence of a hairpin ribozyme (large case lettering) (SEQ ID NO: 1) and its interaction with a substrate RNA (small case lettering) (SEQ ID NO: 2).

FIG. 2 shows the general structure and nucleotide sequence of a hammerhead ribozyme (large case lettering) (SEQ ID NO: 3) and its interaction with a substrate RNA (small case lettering) (SEQ ID NO: 4).

FIG. 3 shows the structure of the RAP6 chimeric hammerhead ribozyme (SEQ ID NO: 5). In the Figure, “pr” indicates propylenediol; the remaining upper case letters (e.g., T, C, G and A) indicate DNA bases; the lower case letters indicate RNA bases; and the underlined lower case letters indicate RNA bases with —OCH₃ attached at the 2-position of that base's sugar moiety.

FIG. 4 shows the structure of the TV2-2 (Est2-2) chimeric hammerhead ribozyme (SEQ ID NO: 6). In the Figure, “pr” indicates propylenediol; the remaining upper case letters (e.g., T, C, G and A) indicate DNA bases; the lower case letters indicate RNA bases; and the underlined lower case letters indicate RNA bases with —OCH₃ attached at the 2-position of that base's sugar moiety.

FIG. 5 shows the pLPR retroviral vector used to clone the ribozyme gene vector library.

FIG. 6 shows Taqman analysis of mRNA target knockdown of the EST2 gene using the RAP2 and TV2-1 (Est2-1) ribozymes.

FIG. 7 shows a radiograph of Northern blot analysis of colon tumor cells and normal colon tissue.

FIG. 8 shows the level of apoptosis in cancer cells (anaplastic transitional cell caricmona urinary bladder cells) when transfected: 1) with the TV2-2 (Est2-2) chimeric hammerhead ribozyme; 2) with the TV2-2 (Est2-2) chimeric hammerhead ribozyme and Fas; 3) with the SR6 (RAP6) chimeric hammerhead ribozyme; 4) with the SR6 (RAP6) chimeric hammerhead ribozyme and Fas; 5) with the TV2-2 (Est2-2) chimeric hammerhead ribozyme and the SR6 (RAP6) chimeric hammerhead ribozyme; and 6) with the TV2-2 (Est2-2) chimeric hammerhead ribozyme and the SR6 (RAP6) chimeric hammerhead ribozyme and Fas.

### DETAILED DESCRIPTION OF INVENTION

The present invention provides isolated nucleic acid molecules encoding ribozymes, each ribozyme having a “substrate binding sequence” that recognizes a target nucleic acid molecule involved in the inhibition of apoptosis induction. The ribozymes of the invention are catalytic RNA molecules that bind to the target nucleic acid molecules and cleave them, thereby impairing their ability to function as inhibitors of apoptosis induction. The ribozymes of the invention are identified and selected by methods described herein. They may be “hairpin” ribozymes, “hammerhead” ribozymes or any other type of ribozyme known in the art.

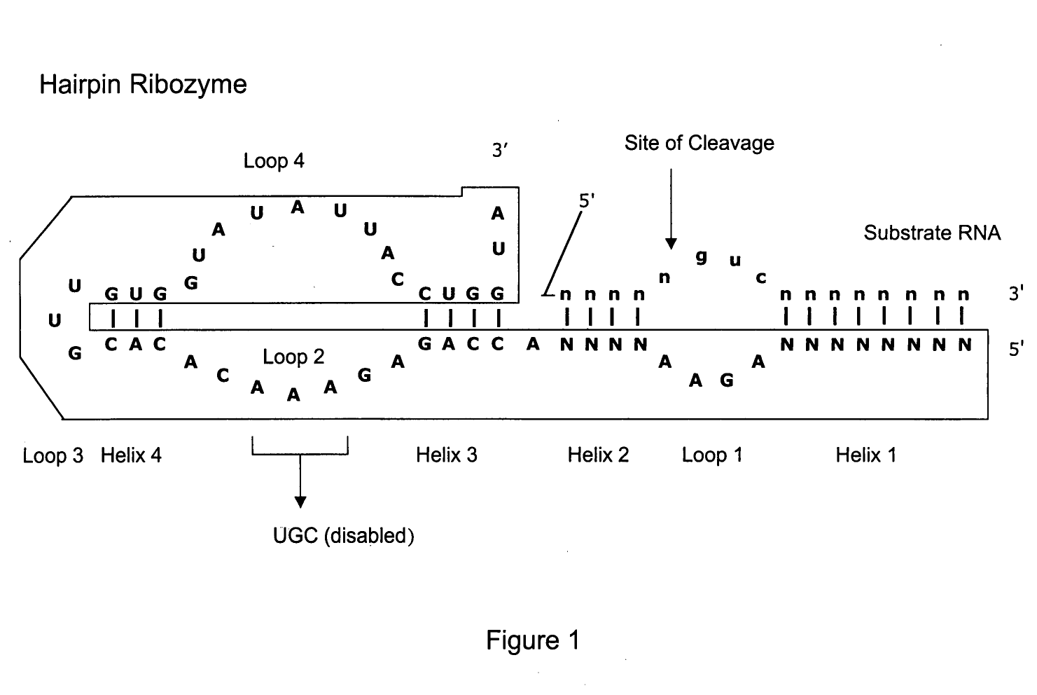

FIG. 1 illustrates the basic structure and nucleotide sequence (shaded, in uppercase letters) of a hairpin ribozyme (SEQ ID NO: 1) and its relationship to the complementary nucleotide sequence (lowercase letters; SEQ ID NO: 2) of a target substrate (N or n=any nucleotide). A hairpin ribozyme consists of a 50 to 54 nucleotide RNA molecule, with the non-substrate binding sequence beginning from the 5′ end at nucleotide position 17. It folds into a 2-dimensional structure that resembles a hairpin, consisting of two helical domains (Helix 3 and 4) and 3 loops (Loop 2, 3 and 4) and two additional helixes (Helix 1 and 2), which form between the ribozyme and the substrate. The length of Helix 2 is fixed at 4 base pairs and the length of Helix 1 typically varies from 6 to 10 base pairs. Recognition of the substrate nucleotides by the ribozyme occurs via Watson-Crick base pairing, with typical substrate recognition sites having the structure 5′-GUC-3′ or 5′-GUA-3′. For maximal activity, the RNA target substrate can contain a GUC in a loop that is opposite Loop 1 with cleavage occurring immediately 5′ of the G as indicated by an arrow. The catalytic, but not substrate binding, activity of a hairpin ribozyme can be disabled by mutating the 5′-AAA-3′ in Loop 2 to 5′-CGU-3′.

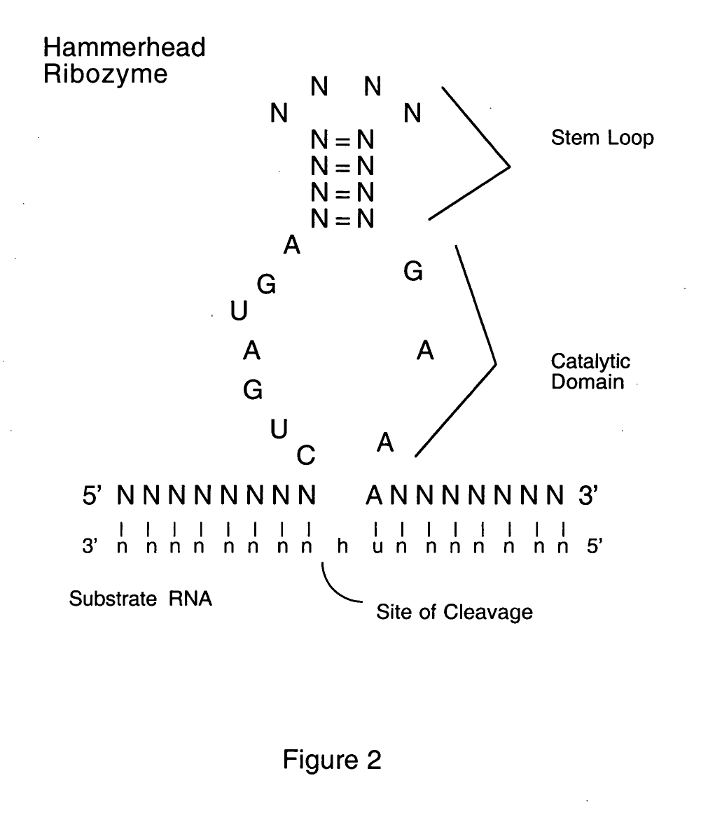

The general structure of a hammerhead ribozyme is shown in FIG. 2 (SEQ ID NO: 3) along with its target RNA sequence (SEQ ID NO: 4). Hammerhead ribozymes suitable for use within the present invention preferably recognize the sequence NUH, wherein N is any of G, U, C, or A and H is C, U, or A. It will be appreciated that the recognition sites of the hairpin ribozyme of the present invention (5′-GUC-3′ or 5′-GUA-3′) are a subset of the hammerhead recognition sites (5′-NUH-3′), such that all hairpin recognition sites are by definition also hammerhead recognition sites, although the converse is not true.

Chimeric hammerhead ribozyme (i.e., RNA/DNA hybrids) are designed to recognize the appropriate NUH sequence for cleavage. Generally, most or all of the binding arms and stem loop comprise DNA. By contrast, generally, the catalytic domain (shown in FIG. 2 between the binding arms and stem loop) comprises RNA.

Modification if the base composition at the stem loop or catalytic domain regions can increase the catalytic activity of the ribozyme, as assayed by in vitro cleavage See WO 00/32765. Modification at the 2-position of the sugar of the base, for example, substituting —OCH₃ at this position of an RNA base, can increase the stability of the ribozyme. Other stabilizing substitutions include —OC₁₋₆Alkyl, —F or other halogens, amino, azido, nitro and phenyl. See U.S. Pat. No. 5,298,612.

The term, “substrate binding sequence” of a ribozyme, as used herein, refers to that portion of the ribozyme which base pairs with a complementary sequence (referred to herein as a “ribozyme sequence tag” or “RST”) of a target nucleic acid. For example, the 16 nucleotides at the 5′ end of the sequence of FIG. 1 (SEQ ID NO:) represent the general formula for a hairpin ribozyme substrate binding sequence. This includes the two arms that form helixes with the target and any necessary nucleotides between these two arms that may be required for the ribozyme function (e.g., AAGA or AAGC). The general formula for the substrate binding sequence of a hammerhead ribozyme is shown in FIG. 2 (SEQ ID NO: 3) with the substrate binding sequence shown aligned with the complementary sequence (RST) of the target RNA (SEQ ID NO: 4).

Because of the basic similarity in the structure of all hairpin ribozymes, they can be modified to obtain a ribozyme having a specific substrate binding sequences of choice. For example, the substrate binding sequence of a “GUC ribozyme”, which cleaves an RNA having the sequence 5′-NNNNN*GUCNNNNNNNN (SEQ ID NO: 7) may be modified to that of a “GUA ribozyme,” which cleaves an RNA having the sequence 5′-NNNNN*GUANNNNNNNN (SEQ ID NO: 8), by changing the base at position 9 from the 5′ end of the substrate binding sequence of the ribozyme. In both of these sequences, N is any of G, U, C, or A; and the asterisk indicates the site where cleavage of the target RNA occurs.

A preferred “GUC hairpin ribozyme” has a substrate binding sequence with the general formula 5′-(N)₍₆₋₁₀₎AGAA(N)₄-3′ (SEQ ID NO: 9), where N can be either G, T, C, or A. A preferred “GUA hairpin ribozyme” has a substrate binding sequence with the general formula 5′-(N)₍₆₋₁₀₎CGAA(N)₄-3′ (SEQ ID NO: 10), where N can be either G, T, C, or A. In the case of the specific ribozyme substrate binding sequences disclosed herein based on these formulas, the sequences also include variations where no more than two nucleotides differ at any of positions 1-5 from the 5′ end of the sequence. This means that one may change one or two bases within the first 5 nucleotides at the 5′ end of a substrate binding sequence and still retain the functional activity of the ribozyme.

Similarly, once the substrate binding sequence of a specific hairpin ribozyme has been identified, it is relatively easy to engineer an equivalent substrate binding sequence for a hammerhead ribozyme. The general structure of the substrate binding sequence of a hammerhead includes six to nine bases at the 5′ end of the ribozyme's binding arm and six to nine bases at the 3′ end of the other binding arm. The following approach, for example, may be used: 1) identify the ribozyme sequence tag (RST) of the RNA target of the specific hairpin ribozyme of interest; 2) specify the first six to nine nucleotides at the 5′ end of the hammerhead as complementary to the first six to nine nucleotides at the 3′ end of the RST; 3) specify the first 5 nucleotides at the 3′ end of the hammerhead as complementary to nucleotides at the 5′ end of the RST; and 4) specify nucleotides at positions 6 and 7 from the 3′ end of the hammerhead as complementary to the RST, while base 8 from the 3′ end is an A.

It should therefore be understood that a ribozyme of the present invention can comprise a) 3, 4, 5, 6, 7, 8 or 9 contiguous bases of any of the ribozyme substrate binding sequences disclosed herein as part of one binding arm of the ribozyme; and b) 3, 4, 5, 6, 7, 8 or 9 contiguous bases of any of the remaining contiguous bases of that ribozyme substrate binding sequence as part of the other binding arm of the ribozyme.

A hammerhead ribozyme can be designed by incorporating sequences of one of the substrate binding sequences disclosed herein, for example bases 2 to 8 and 13 to 16 of the RAP6 substrate binding sequence (SEQ ID NO: 19), or bases 2 to 8 and 13 to 16 of the Est2-2 substrate binding sequence (SEQ ID NO: 97). Alternatively, bases 2 to 8 and 13 to 16 of other substrate binding sequences, for example RAP2 (SEQ ID NO: 17), RAP4: (SEQ ID NO: 18), RAP10 (SEQ ID NO: 20), RAP594 (SEQ ID NO: 21), NHMCZF-4 (SEQ ID NO: 46), Est2-1 (SEQ ID NO: 96), FA5-VR1 (SEQ ID NO: 134) and FA5-VR5 (SEQ ID NO: 138). Indeed, bases 2 to 8 and 13 to 16 of any ribozyme substrate binding sequence disclosed herein can be similarly incorporated into a hammerhead ribozyme.

A preferred chimeric hammerhead ribozyme is SEQ ID NO: 5, which was designed to incorporate base sequences of RAP6 (SEQ ID NO: 19) and to bind to the RAP6 complementary RST (SEQ ID NO: 24). Another preferred chimeric hammerhead ribozyme is SEQ ID NO: 6, which was designed to incorporate base sequences of Est2-2 (SEQ ID NO: 97) and to bind to the Est2-2 complementary RST (SEQ ID NO: 99). As disclosed below in Example 6, these two chimeric hammerhead ribzoymes can be used alone or in combination to facilitate induction of apoptosis in cells, for example cancer cells. Preferably these cells are resistant to apoptosis. Preferably, these ribozymes are used in combination with an apoptosis inducing agent, for example Fas.

In these preferred ribozymes, the stem loop comprises 1,3-propylene-diol linkers. Unreacted, one —OH of the diol is substituted by —O(DMT), where DMT is dimethoxytrityl; and the other —OH is substituted by —P—O—CN-Et)-N(isopropyl)₂. When incorporated into the ribozyme, each —OH is substituted by —O(PO₄).

As used herein, the term “unmodified base” means one of the bases adenine, guanine, cytosine, uracil or thymine attached to the 1-carbon of the sugar (deoxyribose or ribo-furanose), with a phosphate bound to the 5-carbon of the sugar. Bases are bound to each other via phosphodiester bonds between the 3-carbon of one base and the 5-carbon of the next base.

As used herein, the term “modified base” means any base whose chemical structure is modified as follows. Adenine can be modified to result in 6-dimethyl-amino-purine, 6-methyl-amino-purine, 2-amino-purine, 2,6-diamino-purine, 6-amino-8-bromo-purine or 6-amino-8-fluoro-purine. Cytosine can be modified to result in 5-bromo-cytosine, 5-fluoro-cytosine, N,N-dimethyl-cytosine, N-methyl-cytosine, 2-thio-cytosine or 2-pyridone. Guanine can be modified to result in 8-bromo-guanine, 8-fluoroguanine, 2-amino-purine, hypozanthine (inosine), 7-deaza-guanine or 6-thio-guanine. Uracil can be modified to result in 3-methyl-uracil, 5,6-dihydro-uracil, 4-thio-uracil, thymine, 5-bromo-uracil, 5-iodo-uracil or 5-fluoro-uracil. Thymine can be modified to result in 3-methyl-thymine, 5,6-dihydro-thymine, 4-thio-thymine, uracil, 5-bromo-uracil, 5-iodo-uracil or 5-fluoro-uracil. Methods of making such modifications as well as other modifications, such as halogen, hydroxy, amine, alkyl, azido, nitro and phenyl substitutions are disclosed in U.S. Pat. No. 5,891,684; and U.S. Pat. No. 5,298,612. The present invention encompasses sequences where one or more bases are modified.

In addition, the sugar moiety of a base can be modified as disclosed above regarding bases of a hammerhead ribozyme. The present invention encompasses sequences where one or more bases are so modified.

As used herein, the term “nucleic acid” or “nucleic acid molecule” refers to deoxyribonucleotides or ribonucleotides, oligomers and polymers thereof, in either single- or double-stranded form. Unless specifically limited, the term encompasses nucleic acids containing known analogues of natural nucleotides that have similar binding properties as the reference nucleic acid. For example, as disclosed herein, such analogues include those with substitutions, such as methoxy, at the 2-position of the sugar moiety. Unless otherwise indicated by the context, the term is used interchangeably with gene, cDNA and mRNA encoded by a gene.

As used herein, the phrase “a nucleotide sequence encoding” refers to a nucleic acid which contains sequence information, for example, for a ribozyme, mRNA, structural RNA, and the like, or for the primary amino acid sequence of a specific protein or peptide. In reference to a ribozyme, unless otherwise indicated, the explicitly specified encoding nucleotide sequence also implicitly covers sequences that do not materially effect the specificity of the ribozyme for its target nucleic acid. In reference to a protein or peptide, unless otherwise indicated, the explicitly specified encoding nucleotide sequence also implicitly encompasses variations in the base sequence encoding the same amino acid sequence (e.g., degenerate codon substitutions). The invention also contemplates proteins or peptides with conservative amino acid substitutions. The identity of amino acids that may be conservatively substituted is well known to those of skill in the art. Degenerate codons of the native sequence or sequences may be chosen to conform with codon preference in a specific host cell.

As used herein, the term “RNA correlate” of a given DNA sequence means that sequence with “U” substituted for “T.” For example, when every “n” of SEQ ID NO: 19 is “U” (uracil), it is the RNA correlate to SEQ ID NO: 19 when every “n” is “T” (thymine). The present invention encompasses all RNA correlates of every substrate binding sequence and complementary RST disclosed herein.

The term “sequence identity,” when comparing two or more nucleic acid sequences or two or more amino acid sequences, means BLAST 2.0 computer alignment, using default parameters. BLAST 2.0 searching is described, for example by Tatiana et al., FEMS Microbiol. Lett., 174:247-250 (1999), and is available, for example, at http://www.ncbi.nlm.nih.gov/gorf/b12.html.

The term “moderately stringent conditions,” as used herein, means hybridization conditions that permit a nucleic acid molecule to bind to a second nucleic acid molecule that has substantial identity to the sequence of the first. Moderately stringent conditions are those equivalent to hybridization of filer-bound nucleic acid in 50% formamide, 5× Denhart's solution, 5×SSPE, 0.2% SDS at 42° C., followed by washing in 0.2×SSPE, 0.2% SDS at 50° C. “Highly stringent conditions” are those equivalent to hybridization of filer-bound nucleic acid in 50% formamide, 5× Denhart's solution, 5×SSPE, 0.2% SDS at 42° C., followed by washing in 0.2×SSPE, 0.2% SDS at 65° C. Other suitable moderately stringent and highly stringent conditions are known in the art and described, for example, in Sambrook et al., Molecular Cloning: A Laboratory Manual, Cold Spring Harbor Laboratory, New York (1992), and Ansubel et al., Current Protocols in Molecular Biology, John Wiley and Sons, Baltimore Md. (1998).

In general, a nucleic acid molecule that hybridizes to a second one under moderately stringent conditions will have greater than about 60% identity, preferably greater than about 70% identity and, more preferably, greater than about 80% identity over the length of the two sequences being compared. A nucleic acid molecule that hybridizes to a second one under highly stringent conditions will have greater than about 90% identity, preferably greater than about 92% identity and, more preferably, greater than about 95% identity over the length of the two sequences being compared.

As used herein, the term “isolated” when used in conjunction with a nucleic acid or protein, denotes that the nucleic acid or protein has been isolated with respect to the many other cellular components with which it is normally associated in the natural state. For example, an “isolated” gene of interest may be one that has been separated from open reading frames which flank the gene and encode a gene product other than that of the specific gene of interest. Such genes may be obtained by a number of methods including, for example, laboratory synthesis, restriction enzyme digestion or PCR. Likewise, an “isolated” protein may be substantially purified from a natural source or may be synthesized in the laboratory. A “substantially purified” nucleic acid or protein gives rise to essentially one band in an electrophoretic gel, and is at least 85% pure, more preferably at least 95% pure, and most preferably at least 99% pure.

As used herein, the term “expression vector” includes a recombinant expression cassette that has a nucleotide sequence that can be transcribed into RNA in a cell. The cell can further translate transcribed mRNA into protein. An expression vector can be a plasmid, virus, or nucleic acid fragment. Typically, the recombinant expression cassette portion of an expression vector includes the encoding nucleotide sequence to be transcribed (e.g. a ribozyme), operably linked to a promoter, or other regulatory sequence by a functional linkage in cis. In accordance with the present invention, an exression vector comprising a nucleotide sequence encoding ribozymes of the invention can be used to transduce cells suitable as hosts for the vector. Both procaryotic cells including bacterial cells such as E. coli and eukaryotic cells including mammalian cells may be used for this purpose.

As used herein, the term “promoter” includes nucleic acid sequences near the start site of transcription (such as a polymerase binding site) and, optionally, distal enhancer or repressor elements (which may be located several thousand base pairs from the start site of transcription) that direct transcription of the nucleotide sequence in a cell. The term includes both a “constitutive” promoter such as a pol III promoter, which is active under most environmental conditions and stages of development or cell differentiation, and an “inducible” promoter, which initiates transcription in response to an extracellular stimulus, such as a particular temperature shift or exposure to a specific chemical. Promoters and other regulatory elements (e.g., an origin of replication), and/or chromosome integration elements such as retroviral long terminal repeats (“LTRs”), or adeno associated viral (AAV) inverted terminal repeats (“ITRs”), may be incorporated into an expression vector encoding ribozymes of the present invention as described in WO 00/05415 to Barber et al.

As used herein, the term “expresses” denotes that a given nucleic acid comprising an open reading frame is transcribed to produce an RNA molecule. It also denotes that a given nucleic acid is transcribed and translated to produce a polypeptide. Although the term may be used to refer to the transcription of a ribozyme, a ribozyme typically is not translated into a protein since it functions as an active (catalytic) nucleic acid.

As used herein, the term “gene product” refers either to the RNA produced by transcription of a given nucleic acid or to the polypeptide produced by translation of a given nucleic acid.

As used herein, the term “transduce” denotes the introduction of an exogenous nucleic acid molecule (e.g., by means of an expression vector) inside the membrane of a cell. Exogenous DNA may or may not be integrated (covalently linked) into chromosomal DNA making up the genome of the cell. The exogenous DNA may be maintained on an episomal element, such as a plasmid. In eukaryotic cells, a stably transduced cell is generally one in which the exogenous DNA has become integrated into the chromosome so that it is inherited by daughter cells through chromosome replication, or one which includes stably maintained extrachromosomal plasmids. This stability is demonstrated by the ability of the eukaryotic cell to establish cell lines or clones comprised of a population of daughter cells containing the exogenous DNA.

The term “transfection,” as used herein, means the genetic modification of a cell by uptake of an exogenous nucleic acid molecule (e.g., by means of an expression vector).

As used herein, the term “ribozyme gene vector library” denotes a collection of ribozyme-encoding genes, typically within expression cassettes, in a collection of viral or other vectors. The vectors may be naked or contained within a capsid. Propagation of the ribozyme gene vector library can be performed as described in WO 00/05415 to Barber et al. The ribozyme-encoding genes of a ribozyme gene vector library, after transduction and transcription in appropriate cells, produce a collection of ribozymes.

In accordance with a preferred embodiment of the present invention, a random retroviral ribozyme gene vector library was propagated, as described in Example 1 below. The retroviral ribozyme gene vector library was then used to transduce DLD-1 colon carcinoma cells, as described in Example 2 below. DLD-1 colon carcinoma cells were chosen because they were found to be resistant to apoptosis (as indicated by antibody triggering of Fas (CD95), which facilitates induction of apoptosis). The cells were then subjected to apoptosis induction, and apoptotic cells were identified and separated. Genomic DNA was isolated from the apoptotic cells, and the ribozyme genes were rescued.

As described in Example 2, three rounds of successive vector transduction, apoptosis induction, cell selection and ribozyme gene rescue were performed. After the third round of selection, the inventors found that approximately 5-6% of the cells that had been transduced with the ribozyme gene vector library had entered apoptosis as compared to 1-2% of the control cells.

Upon analysis of the nucleotide sequences of the ribozyme genes, the inventors observed that five ribozyme substrate binding sequences were predominant (see Example 2, Tables 1-5). The validity of these five ribozyme substrate binding sequences for facilitating apoptosis induction was then confirmed, as described in Example 2 below.

In accordance with a preferred embodiment of the present invention, genes involved in the inhibition of apoptosis induction were identified, as more fully described in Example 3 below. Since ribozymes recognize their cognate targets by sequence complementarity, the substrate binding sequence of the ribozymes of the present invention (see Tables 1-5) were used to identify the ribozyme sequence tags (RSTs) of the RNA that were cleaved by the ribozymes of the present invention and which were involved in the inhibition of apoptosis induction. The identified RSTs of the cognate targets of the ribozymes of the present invention are also set forth in Tables 1-5 below.

The present invention provides isolated molecules comprising any of the complemetary RSTs listed in Tables 1 to 5 below; the complementary RST to NHMCZF-4, Est2-1, Est2-2, FA5-VR1 or FA5-VR5; or the complementary RSTs listed in Tables 7 and 9 below. In addition, any of these molecules can be 150 bases or shorter, 125 bases or shorter, 100 bases or shorter, 90 bases or shorter, 80 bases or shorter, 70 bases or shorter, 60 bases or shorter, 50 bases or shorter, 40 bases or shorter, 30 bases or shorter, 25 bases or shorter, or 16 bases in length. These molecules can inhibit induction of apoptosis or, alternatively, be used to identify agents that facilitate induction of apoptosis.

Once the RSTs of the cognate targets of the ribozymes of the present invention were identified, the inventors conducted a search of the various public gene databases (such as the nr (nonredundant) database, the EST (Expressed Sequence Tag)-Human database, the EST-Mouse database, the dbEST database, and the like) using the “BLAST” program (Basic Local Alignment Search Tool; http://www.ncbi.nlm.nih.gov/BLAST/), to identify genes and gene fragments containing one or more of the RST sequences (Tables 1-5) identified in accordance with the present invention. As more fully described in Example 3 below, this search disclosed several complete matches with gene sequences in the public databases.

In accordance with the present invention, the involvement in apoptosis inhibition of the gene sequences that matched the identified RSTs was then validated (see Example 3 below). Based upon the known sequences of the identified genes, “validation” ribozymes were constructed having substrate binding sequences complementary to RST's of the identified genes, and these were engineered for expression in retroviral vectors in accordance with the present invention. The validation ribozymes were then expressed in cells and analyzed for their ability to facilitate apoptosis induction in accordance with the present invention. The results are set forth in Example 3.

The present invention provides additional nucleic acid sequences that contain EST2 (SEQ ID NO: 31), which is involved in inhibiting apoptosis induction (see Example 3). Specifically included in the present invention and described in Example 5, are sequences comprising or consisting of a “contig,” or contiguous sequence, of about 1.7 kb (SEQ ID NO: 146), about 2 kb (SEQ ID NO: 148), about 2.3 kb (SEQ ID NO: 150), about 2.6 kb (SEQ ID NO: 152), about 3.4 kb (SEQ ID NO: 155), about 4.1 kb (SEQ ID NO: 157) and about 5.5 kb (SEQ ID NO: 166). Also provided are the RNA correlate of each of these sequences as well as compounds having 90%, 91%, 92%, 93%, 94%, 95%, 96%, 97%, 98% and, most preferably, 99% sequence identity with any of these sequences. Also provided are compounds that hybridize under moderately or, more preferably, stringent conditions with any of these sequences.

The present invention also provides a method of facilitating the induction of apoptosis in a cell resistant to induction of apoptosis. This method comprises introducing a ribozyme of the invention into a cell, for example one resistant to apoptosis. This method can comprise transducing the apoptosis induction resistant cell with an expression vector encoding a ribozyme with a substrate binding sequence of the present invention. Alteratively, as shown for instance in Example 6, a ribozyme of the invention can be introduced into a cell directly, i.e., without using a vector.

These ribozymes of the invention include those comprising the substrate binding sequences listed in Tables 1-5 (SEQ ID NOS: 17-21); NHMCZF-4 (SEQ ID NO: 46); listed in Tables 7 and 9 (SEQ ID NOS: 50-72 and 100-112); listed in Table 8 (SEQ ID NOS: 96-97); FA5-VR1 (SEQ ID NO: 134); and FA5-VR5 (SEQ ID NO: 138), and any other hairpin or hammerhead ribozyme comprising a substrate binding sequence designed as described herein and which binds to and cleaves any of the genes disclosed herein, including NHMCZF (GenBank Accession No. AL096880; (SEQ ID NO: 27)), FLJ22165 (GenBank Accession No. AK025818; (SEQ ID NO: 40)), FAPP2 (SEQ ID NO: 42) or human PATZ (SEQ ID NO: 29).

The present invention also provides amino acid sequences encoded by the nucleic acid sequences disclosed herein. For example, provided is a compound comprising or consisting of SEQ ID NO: 158, which is the amino acid sequence encoded by bases 3-962 of the 4.1 kb contig (SEQ ID NO: 157). Also provided is a compound comprising or consisting of SEQ ID NO: 167, which is the amino acid sequence encoded by bases 1-999 of the 5.5 kb contig (SEQ ID NO: 166). Also provided are compounds that have 80%, 85%, 88%, 90%, 92%, 94%, 95%, 96%, 97%, 98% and, most preferably, 99% amino acid identity with these amino acid sequences (SEQ ID NOS: 158, 167). Additionally provided are compounds that comprise or consist of 25, 20, 15, or 10 or more contiguous amino acids of these amino acid sequences (SEQ ID NOS: 158, 167).

The present invention also provides additional amino acid sequences encoded by the nucleic acid sequences disclosed herein. For example, provided is a compound comprising or consisting of SEQ ID NOS: 169, 170 and 171, which are the amino acid sequence encoded by NHMCZF (GenBank Accession No. AL096880; (SEQ ID NO: 27), FAPP2 (SEQ ID NO: 42) and human PATZ (SEQ ID NO: 29), respectively. Also provided are compounds that have 80%, 85%, 88%, 90%, 92%, 94%, 95%, 96%, 97%, 98% and, most preferably, 99% amino acid identity with these amino acid sequences (SEQ ID NOS: 169, 170 or 171). Additionally provided are compounds that comprise or consist of 25, 20, 15, or 10 or more contiguous amino acids of these amino acid sequences (SEQ ID NOS: 169, 170 or 171).

The compounds containing the amino acid sequences described above can be used to inhibit induction of apoptosis in a cell. Alternatively, these compounds can be sued to identify agents that promote induction of apoptosis in a cell.

Accordingly, the present invention provides methods of identifying agents that promote induction of apoptosis in a cell. Such method includes: 1) assessing the binding capability of the agent with a) a target molecule containing an RST of the invention as described herein; or b) a target molecule containing an amino acid sequence described above; and 2) introducing the agent into the cell and measuring the level of apoptosis, where an increase in the level indicates that the agent promotes induction of apoptosis.

In accordance with the present invention, cells that are resistant to apoptosis induction may be rendered susceptible to apoptosis induction by the method described hereinabove, and the cells may then be contacted with an apoptosis inducing agent so as to induce apoptosis in the cell. This method is particularly useful for treating cancer cells such as leukemia cells, as well as other cancer cells such as bladder brain, lung, colon, pancreatic, breast, ovarian, cervical, liver pancreatic, stomach, lymphatic, prostate and the like. Any of a variety of well-known apoptosis inducing agents can be used for this purpose. A preferred apoptosis inducing agent is one that triggers a “death receptor” type cell surface protein (Baker et al. (1996) Oncogene. 12:1-9), which includes Fas, TNF-alpha receptor, the TRAIL receptor, and the like. A particularly preferred apoptosis inducing agent is one that triggers the FAS receptor, such as an antibody to the FAS receptor as described in the Examples or soluble FAS ligand (see U.S. Pat. No. 6,042,826 to Caligiuri et al.). Other suitable apoptosis inducing agents include adamantyl derivatives (see U.S. Pat. No. 6,127,415 to Pfahl et al.), 2-nitroimidazole derivatives (see U.S. Pat. No. 5,929,014 to Ohyama), benzamidine riboside (see U.S. Pat. No. 5,902,792 to Jayaram), branched apogenic peptide (see U.S. Pat. No. 5,591,717 to Rojko et al.), chemotherapeutic agents such as 5-FU, cisplatin, vincristine, methotrexate, doxirubicin, and the like.

The present invention also provides a method of facilitating the induction of apoptosis in a cell resistant to induction of apoptosis, comprising reducing the level of a protein expressed in the cell which is involved in inhibiting apoptosis induction, and then contacting the cell with an apoptosis inducing agent, such as the agents described above, to induce the cell to undergo apoptosis. The step of reducing the level of the protein involved in apoptosis inhibition preferably is carried out by reducing the level of the RNA in the cell encoding the protein. In accordance with one embodiment of the present invention, this is accomplished by transducing the cell with an expression vector encoding a ribozyme having a substrate binding sequence that enables the ribozyme to cleave the RNA encoding the protein. Any of the ribozymes disclosed herein, which have been shown to be effective in facilitating the induction of apoptosis, may be used for this purpose.

In accordance with another embodiment of the present invention, the step of reducing the level of the target protein in the cell can be accomplished by contacting the cell with antisense compounds which are complementary to any portion of the substrate binding sequences of the ribozymes disclosed herein.

The antisense compounds that may be used in connection with this embodiment of the present invention preferably comprise between about 8 to about 30 nucleobases (i.e., from about 8 to about 30 linked nucleosides), more preferably from about 12 to about 25 nucleobases, and may be linear or circular in configuration. They may include oligonucleotides containing modified backbones or non-natural internucleoside linkages. Preferred modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Methods of preparing antisense compounds are well known in the art (see, for example, U.S. Pat. No. 6,210,892).

In accordance with yet another embodiment of the present invention, the specific activity of the protein expressed in the cell (such as inhibiting the induction of apoptosis by Fas triggering in DLD-1 colon carcinoma cells) may be reduced further by treating the cell with an agent that binds to the protein and inhibits its activity. Agents suitable for this purpose can be peptides, nucleic acids, organic compounds, and the like. Bioassays for selecting protein binding agents that modulate protein activity are well known in the art (see, e.g., U.S. Pat. No. 5,618,720).

The present invention also provides a method of inhibiting the growth of a cancer in a subject, the method comprising administering to the subject an effective amount of an expression vector comprising a sequence of nucleotides that encodes a ribozyme having a substrate binding sequence disclosed herein. The expression vector is preferably administered in combination with a suitable carrier. After the vector has been administered, the ribozyme is expressed in the cells and apoptosis induction facilitated as described herein. The subject may optionally be treated with an apoptosis inducing agent, as disclosed herein, to further induce apoptosis and reduce the growth of the tumor.

Administration of the vector or the apoptosis inducing agent can be by any suitable route including oral, sublingual intravenous, subcutaneous, transcutaneous, intramuscular, intracutaneous, and the like. Any of a variety of non-toxic, pharmaceutically acceptable carriers can be used for formulation including, glucose, lactose, gum acacia, gelatin, mannitol, starch paste, magnesium trisilicate, talc, corn starch, keratin, colloidal silica, potato starch, urea, dextrans, and the like. The formulated material may take any of various forms such as injectable solutions, sterile aqueous or non-aqueous solutions, suspensions or emulsions, tablets, capsules, and the like.

As used herein, the phrase “effective amount” refers to a dose of the deliverable sufficient to provide circulating concentrations high enough to impart a beneficial effect on the recipient, which is inhibition of cancer growth. With the vector deliverable, the concentration of vector administered should be sufficient to transform enough of the target cells. With the apoptosis inducing deliverable, the concentration should be sufficient to induce apoptosis in a sufficient number of the target cells.

The specific therapeutically effective dose level for any particular subject and deliverable depends upon a variety of factors including the disorder being treated, the severity of the disorder, the activity of the specific compound administered, the route of administration, the rate of clearance of the specific compound, the duration of treatment, the drugs used in combination or coincident with the specific compound, the age, body weight, sex, diet and general health of the patient, and like factors well known in the medical arts and sciences. Dosage levels typically range from about 0.001 up to 100 mg/kg/day; with levels in the range of about 0.05 up to 10 mg/kg/day.

Methods for preparing oligonucleotide probes known in the art may be effectively used for preparing the oligonucleotide probes of the present invention. See, for example, Beaucage and Carruthers (1981) Tetrahedron Lett. 22:1859-1862; Matteucci et al. (1981) J. Am. Chem. Soc., 103:3185; Sambrook et al. (1989) Molecular Cloning: A Laboratory Manual (2d ed.), Vols. 1-3, Cold Spring Harbor Laboratory; F. Ausubel et al. (ed.) (1987) Current Protocols in Molecular Biology, Greene Publishing and Wiley-Interscience, New York (1987); Hames and Higgins (eds.) (1985) Nucleic Acid Hybridization, A Practical Approach, IRL Press; Gall and Pardue (1969) Proc. Natl. Acad. Sci. USA, 63:378-383; and John et al. (1969) Nature 223:582-587.

Typically, the probes used to detect hybridization are labeled to facilitate detection but the target nucleic acid may be labeled instead. Probes or nucleic acid targets may be labeled by any one of several methods typically used to detect the presence of hybridized polynucleotides. The most common method of detection is the use of autoradiography with ³H, ¹²⁵I, ³⁵S, ¹⁴C, or ³²P-labeled probes, or the like. Other labels include ligands which bind to labeled antibodies, fluorophores, chemiluminescent agents, enzymes, and antibodies which can serve as specific binding pair members for a labeled ligand. See, for example, Tijssen, P., “Practice and Theory of Enzyme Immunoassays” in Burdon, R. H., van Knippenberg, P. H. (eds.) (1985) Laboratory Techniques in Biochemistry and Molecular Biology, Elsevier, pp. 9-20.

The present invention also provides an antibody with binding specificity for a protein that inhibits the induction of apoptosis; the antibody may also be used to detect the level or activity of a polypeptide involved in the inhibition to apoptosis induction. In a preferred embodiment, the antibody has binding specificity for a protein or peptide (i.e., amino acid sequence) encoded by the genes or nucleic acid sequences disclosed herein.

As used herein, the term “antibody” comprises two heavy chains and two light chains that associate to form two binding sites in each antibody molecule. The term also contemplates fragments of antibodies such as Fab′2 fragments and fragments with a single binding site such as Fab′ Fv sFv, and the like. The term includes a monoclonal antibody, a polyclonal antibody, or a collection of polyclonal antibodies such as is present in the antiserum of an immunized animal.

As used herein, the phrase “binding specificity,” in relationship to an antibody that binds to a protein or peptide, refers to a binding reaction which is determinative of the presence of the protein in the presence of a heterogeneous population of proteins and other biologics. Thus, under designated immunoassay conditions, the specified antibody binds to a particular protein and does not bind significantly to other proteins present in the sample.

Methods of producing polyclonal and monoclonal antibodies are known to those of skill in the art. See, e.g., Coligan (1991) Current Protocols in Immunology Wiley/Greene, N.Y.; and Harlow and Lane (1989) Antibodies: A Laboratory Manual, Cold Spring Harbor Press, NY; Stites et al. (eds.) Basic and Clinical Immunology (4th ed.) Lange Medical Publications, Los Altos, Calif.; Huse et al. (1989) Science 246:1275-1281; and Ward et al. (1989) Nature 341:544-546, and references cited therein.

The diagnostic methods described herein are applicable to the identification of cancer cells resistant to apoptosis induction present in, for example, solid tumors (carcinomas and sarcomas) such as, for example, breast cancer, ovarian cancer and prostate cancer. Such methods include the detection of a nucleic acid encoding a molecular product having an RST identified herein as involved in inhibiting apoptosis induction.

Various qualitative and quantitative assays to detect altered expression or structure of a nucleic acid molecule in a sample are well known in the art, and generally involve hybridization of the target sequence to a complementary primer or probe (which may be referred to as a reagent). Such assays include, for example, in situ hybridization, which can be used to detect altered chromosomal location of the nucleic acid molecule, altered gene copy number, or altered RNA abundance, depending on the format used. Other assays include, for example, RNA blots and RNase protection assays, which can be used to determine the abundance and integrity of RNA; DNA blots, which can be used to determine the copy number and integrity of DNA; SSCP analysis, which can detect single point mutations in DNA, such as in a PCR or RT-PCR product; and coupled PCR, transcription and translation assays, such as the Protein Truncation Test, in which a mutation in DNA is determined by an altered protein product on an electrophoresis gel. Further assays include methods known in the art for genotyping, for example, by RFLP analysis or by determining specific SNPs. An appropriate assay format and reagent to detect an alteration in the expression or structure of an apoptosis induction resistance regulator nucleic acid molecule can be determined by one skilled in the art depending on the alteration one wishes to identify.

The invention also includes a high throughput drug discovery method using ribozyme transduced cells and chip array technology to identify compounds that modulate apoptosis induction regulatory activity. By combining array technology with ribozyme knockdown, drugs can be rapidly screened for effects on a given pathway. Once the expression profile leading to a given phenotype is determined, additional arrays can be generated with the relevant regulated EST sequences. These can be screened with mRNA from drug treated cells. Profiles matching the ribozyme treated profile can be identified. Treatment with the drugs identified in this way can be expected to give the desired phenotype.

This methodology allows the linking of the function of these target genes to the desired phenotype, i.e., modulation of apoptosis induction. Small molecule drugs, ribozyme drugs, or antibody drugs can be identified by those skilled in the art that inhibit the activity of these gene targets resulting, for example, in reduced resistance to apoptosis induction. The gene targets can be used to develop high throughput assays that can be screened with existing small molecule libraries. In addition, genes which express a surface or secreted protein can be targets for antibody development. Antibodies specific for the gene product can be generated preferably in transgenic mouse systems to generate human antibodies. Furthermore, chimeric ribozyme drugs targeting these apoptosis induction resistance regulators can be designed as explained above.

As used herein, “a target molecule responsive to the activity of the apoptosis induction regulator” means that the apoptosis induction regulator either binds or chemically modifies the target molecule that exists in a cell. For example, if the apoptosis induction regulator has DNA binding activity for use in, for example, transcriptional gene regulation, the target molecule responsive to this activity is a nucleic acid comprising a sequence of nucleotides recognized and bound by the particular apoptosis induction regulator. If the apoptosis induction regulator has protein kinase activity, due for example to having a serine/threonine kinase domain, the target molecule responsive to this activity is a protein having an appropriate serine or threonine kinase recognition site. Likewise, for activity as a protease, the target molecule responsive is a protein cleaved by the apoptosis induction regulator.

When the apoptosis induction regulator activity to be measured for drug screening is DNA binding, such binding can be determined by assaying the expression of a reporter gene that is operatively linked to the nucleic acid element. In this case, an increase in the amount of expression or activity of the reporter gene in the presence of a test compound compared to the absence of the test compound indicates that the compound has apoptosis induction regulator DNA binding inhibitory activity. The magnitude of the increase in expression activity will correlate with the apoptosis induction regulator inhibitory activity of the test compound. Exemplary reporter genes include apoptosis induction, EGFP and hygromycin resistance gene.

As used herein, the term “nucleic acid element” when used in reference to regulation of apoptosis induction expression refers to a nucleic acid region that modulates apoptosis induction expression. Exemplary nucleic acid elements are the apoptosis induction 5′ promoter and regulatory region or other transcriptional regulation regions, and translational regulatory regions of the transcribed mRNA. Generally, the nucleic acid element will be the 5′ promoter and regulatory region.

Similarly, compounds that increase or enhance the activity of apoptosis induction regulator also can be identified. A test compound added to a sample containing an apoptosis induction regulator and a nucleic acid element modulated by an apoptosis induction regulator which decreases apoptosis induction activity or the amount or rate of expression of apoptosis induction or a reporter gene operatively linked to the nucleic acid element compared to the absence of the test compound indicates that the compound increases the activity of the apoptosis induction regulator. Therefore, the invention provides a method of identifying compounds that modulate the activity of an apoptosis induction regulator.

A reaction system for identifying a compound that inhibits or increases apoptosis induction regulator activity can be prepared using essentially any sample, material or components thereof that contains an apoptosis induction regulator. An apoptosis induction regulator containing sample used for such methods can be, for example, in vitro transcription or translation systems using, for example, nucleic acid derived from the apoptosis induction gene-of a normal or tumor cell or a hybrid construct linking the nucleic acid element modulated by an apoptosis induction regulator to a reporter gene. Alternatively, nucleic acids and proteins obtained from normal cells can also be used since apoptosis induction regulators can also act in normal cells. The apoptosis induction regulator-containing sample can additionally be derived from cell extracts, cell fractions or, for example, in vivo systems such as cell culture or animal models which contain a nucleic acid element modulated by an apoptosis induction regulator. The expression levels or activity of apoptosis induction or the reporter gene can be measured in the reaction system to determine the modulatory effect of the test compound on the apoptosis induction regulator. Such measurements can be determined using methods described herein as well as methods well known to those skilled in the art.

Briefly, the apoptosis induction regulator source is combined with a nucleic acid element or protein modulated by an apoptosis induction regulator as described above and incubated in the presence or absence of a test compound. The expression levels or activity of apoptosis induction or the reporter gene in the presence of the test compound is compared with that in the absence of the test compound. Those test compounds which provide an increase in expression levels or activity of apoptosis induction or the reporter gene of at least about 20% are considered to be apoptosis induction regulator activators, or agonists, and are potential therapeutic compounds for the treatment of neoplastic diseases such as cancer. Similarly, those compounds which decrease expression levels or activity of apoptosis induction regulator or the reporter gene by about 20% or more are considered to be compounds which decrease the activity of an apoptosis induction regulator, or apoptosis induction regulator antagonists. Such antagonists can be used as therapeutics, for example, to promote cell growth or cell survival in transplanted or explanted cells which are subsequently transplanted. Compounds identified to modulate apoptosis induction regulator activity can, if desired, be subjected to further in vitro or in vivo studies to corroborate that they affect the activity of an apoptosis induction regulator toward the apoptosis induction expression or activity.

Suitable test compounds for the above-described assays can be any substance, molecule, compound, mixture of molecules or compounds, or any other composition which is suspected of being capable of inhibiting apoptosis induction regulator activity in vivo or in vitro, for example, compounds with cell proliferation-inhibiting activity. The test compounds can be macromolecules, such as biological polymers, including proteins, polysaccharides and nucleic acids. Sources of test compounds which can be screened for apoptosis induction regulator inhibitory activity include, for example, libraries of small organic molecules, peptides, polypeptides, DNA, and RNA. Additionally, test compounds can be pre-selected based on a variety of criteria. For example, suitable test compounds can be selected as having known inhibition or enhancement activity with respect to cell proliferation. Alternatively, the test compounds can be selected randomly and tested by the screening methods of the present invention. Test compounds can be administered to the reaction system at a single concentration or, alternatively, at a range of concentrations to determine, for example, the optimal modulatory activity toward the apoptosis induction regulator.

The activity of an apoptosis induction regulator for which drug screening is desired can be a protein kinase activity. For example, apoptosis induction regulators that have a serine/threonine kinase domain may be used for drug screening where the activity which is modulated is a protein kinase activity. Protein kinase assays are well known to those skilled in the art (see, e.g., U.S. Pat. Nos. 5,538,858 and 5,757,787; Anal. Biochem, 209:348-353, (1993)).

The activity of an apoptosis induction regulator for which drug screening is desired also can be GTP binding activity. For example, apoptosis induction regulators that have a GTP binding site may be used for drug screening where the activity which is modulated is GTP binding. Apoptosis induction regulators that have GTP-binding activity may regulate cell growth such as through regulating apoptosis induction expression, or may have affects on cell cycle control, protein secretion, and intracellular vesicle interaction. GTP binding assays are well known to those skilled in the art (see, e.g., U.S. Pat. Nos. 5,840,969 to Hillman et al.).

The activity of an apoptosis induction regulator for which drug screening is desired also can be hormone binding activity. For example, apoptosis induction regulators that have a hormone binding site may be used for drug screening where the activity which is modulated is hormone binding. Hormones that bind to an apoptosis induction regulator may be steroid hormones such as estrogen or a protein based hormone. Receptor hormone binding assays including receptor estrogen binding assays are well known to those skilled in the art (see, e.g., U.S. Pat. Nos. 6,204,067 to Simon et al.).

The present invention also provides kits for carrying out the methods of the present invention. Such kits include one or more reagents of the invention such as antibodies or oligonucleotide probes specific for polypeptides or genes, respectively, involved in inhibition of apoptosis induction. Such agents may be detectably labeled using an appropriate enzyme, dye, radioisotope, and the like. The kits also may include additional reagents specific for binding to the reagents of the invention as well as necessary chemicals and buffers.

The following examples are intended to illustrate but not limit the present invention.

### EXAMPLES

### Example 1

### Preparation of a Retroviral Random Ribozyme Gene Vector Library

This example describes the preparation of a retroviral random ribozyme gene vector library, the first step in the method for selecting and identifying ribozymes having substrate recognition sequences involved in facilitating apoptosis induction. The library was prepared essentially as described in WO 00/05415 (Barber et al.). The plasmid-based retroviral ribozyme library was created in vector pLPR. Vector pLPR-1 kb contains: 1) 5′ and 3′ long terminal repeats (LTR) of the Moloney retroviral genome; 2) transcription cassette for the ribozyme genes via tRNAval promoter with a 1 kb stuffer insert at the site intended for the ribozyme gene; and 3) SV40 promoter driving puromycin resistance. In this design, the stuffer insert was removed and replaced by the random ribozyme library insert, with transcription under control of the tRNAval promoter.

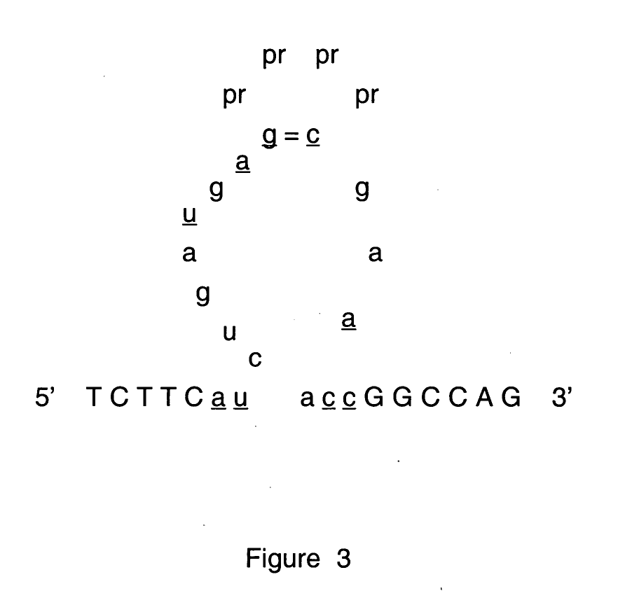

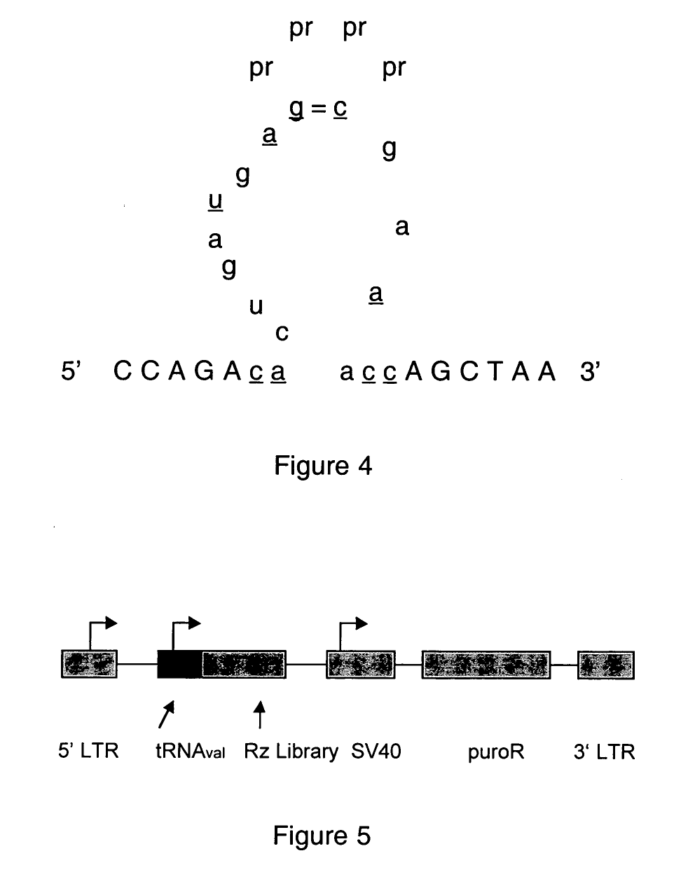

The pLPR-1 kb vector (see FIG. 3) was prepared by digesting plasmid pLPR overnight at 37° C. with BamH1, phenol:chloroform extracted and ethanol precipitated. The resuspended DNA was then digested overnight at 37° C. with MluI. This double digestion excises the 1 kb stuffer fragment. The resultant 6 kb plasmid vector DNA fragment was purified by agarose gel electrophoresis.

The random ribozyme library inserts were prepared from three oligonucleotides, which were synthesized and annealed in annealing buffer (50 mM NaCl, 10 mM Tris pH 7.5, 5 mM MgCl₂) at a molar ratio of 1:3:3 (oligo1:oligo2:oligo3) by heating to 90° C. followed by slow cooling to room temperature. The three oligonucleotides had the following sequences:

| | |
|---|---|
| Oligo1: | |
| 5′-pCGCGTACCAGGTAATATACCACGGACCGAA | (SEQ ID NO: 11) |
| GTCCGTGTGTTTCTCTGGTNNNNTTCTNNNNNNN | |
| NGGATCCTGTTTCCGCCCGGTTT-3′ | |
| Oligo2: | |
| 5′-pGTCCGTGGTATATTACCTGGTA-3′ | (SEQ ID NO: 12) |
| Oligo3: | |
| 5′-pCGAAACCGGGCGGAAACAGG-3′ | (SEQ ID NO: 13) |

Random incorporation of A, T, C and G nucleotides at the positions represented as N in oligo 1, was achieved by premixing A, T, C and G reagents at every N position in the oligonucleotide synthesis. The ribozyme insert library formed by annealing the three oligonucleotides (SEQ ID NOS: 11, 12, 13) thus contains 8 positions with random nucleotides corresponding to helix 1 of the ribozyme, and 4 random positions with random nucleotides corresponding to helix 2 of the ribozyme (see FIG. 1).

A pLPR-1 kb vector DNA fragment was ligated overnight to the random ribozyme insert library using 0.5 pmole of the vector, an 8-fold molar excess of annealed oligonucleotides and 10 units of T4 DNA ligase. The resulting library of vectors, designated pLPR-library were electroporated into ultracompetent DH12S bacteria. A total of 5×10⁷ bacterial colonies containing the retroviral plasmid ribozyme library were obtained.

Bacterial colonies containing the retroviral plasmid ribozyme library were pooled in aliquots as a master stock and frozen at −80° C. Working stocks were made by culturing 1 ml of the master stock in 60 ml LB media overnight at 30° C. A 1 ml aliquot of the working stock was used to make a 500 ml bacterial culture by incubation at 30° C. overnight. Retroviral DNA was then extracted from the 500 ml culture and used to prepare viral vector for the library selection.

A viral vector was produced from the ribozyme library plasmid using a triple transfection technique. In this approach, CF2 cells were seeded at a concentration of 3.5×10⁴ cells/cm². The next day, 2.2×10⁸ CF2 cells were incubated for 6 hours in 665 ml of serum-free medium transfection media containing 20 mg of a triple plasmid mixture complexed with 12 ml of a cationic lipid (TransIT-LT1; Pan Vera Corporation). The plasmid mixture contained a 2:3:1 ratio of the ribozyme gene library plasmid (or control ribozyme plasmid), a plasmid encoding the moloney-murine virus gag-pol genes, and a plasmid encoding the vesicular stomatitis virus-G gene. Cell supernatant containing retroviral particles was collected every 24 hours beginning on day 2 following transfection. The viral containing supernatant was filtered through 0.4 μm filters and titred in a standard assay using HT1080 cells (see WO 00/05415 to Barber et al.).

Following the cloning of the randomized hairpin ribozyme genes into pLPR, the “randomness” of the plasmid library was evaluated as described in WO 00/05415 to Barber et al. The frequencies of the four nucleotides, with 95% confidence limits, in the random positions were calculated to be G: 22.3±6.1, A: 31.9±7.0, T: 27.3±7.8 and C: 18.01±15.1. Since the expected frequency for each base is 25%, each base appears to be randomly represented (except for C, which may be slightly lower than expected). These variations most likely result from the unbalanced incorporation of nucleotides during the chemical synthesis of the oligonucleotides and could reduce the complexity of the library.

For a functional evaluation of the library's complexity, in vitro cleavage was utilized to determine if ribozymes that target known RNA substrates were present in the library pool. This involved in vitro transcribing of the entire ribozyme library in one reaction and then testing the pool's ability to cleave a variety of different RNA substrates of both cellular and viral origin. Six out of seven known RNA targets were properly and efficiently cleaved by the in vitro transcribed library. This qualitative analysis suggested a significantly complex library of ribozyme genes and the lack of cleavage of one target out of seven may reflect the slight non-randomness suggested by the base composition described above.

### Example 2

### Introduction of the Random Ribozyme Gene Vector Library into Mammalian Cells and Selection of Apoptosis Induction Relevant Nucleotide Sequences

This example describes a method for identifying ribozymes involved in apoptosis induction. The pLPR-library vector described in Example 1 and a control vector, pLPR-TL3, were used to transduce DLD-1 colon carcinoma cells (ATCC, Bethesda Md.). The control vector differs from the pLPR-library vector (see FIG. 2) in having an HCV ribozyme control gene in place of the ribozyme library gene.

For transduction, DLD-1 cells were grown to about 70% confluency in T225 flasks (about 6×10⁷ cells). Transduction of the cells was accomplished by incubating them for 24 hours at 37° C. with retroviral vector coding for the library at a multiplicity of infection (MOI) of 1.

After incubation with retroviral vector, the transduction medium was removed by aspiration and replaced with growth medium containing puromycin (2 μg/ml). The next day, cells were re-fed with media containing 2 μg/ml puromycin. The cells were maintained in selection medium for 10-14 days in order to select for stable integration of the retroviral vector. During the course of this selection the cells were re-fed every three days.

After stable selection, the cells were subjected to induction of apoptosis by incubation for 18 hours with purified IgM ligating antibody to CD95 (clone 11, PanVera) added at 160 ng/ml. Apoptotic cells were then identified by a dual staining protocol. In a first step, following induction, cells were removed by trypsin, washed 2× with PBS, suspended in binding buffer and then stained with Annexin-V-FITC/PI, essentially as described by the manufacturer (Boerhinger/Manheim). Annexin-V binds to phosphatidyl serine, which translocates from the inner cell membrane space to the outer cell membrane surface early in apoptosis. Concurrent staining with propidium iodide (PI), a DNA stain, also was used to identify and exclude necrotic cells from the population of cells undergoing apoptosis.

Subsequently, the TUNEL assay (Roche), which is believed to provide less variability in the identification of apoptotic cells, was used. Following staining (or TUNEL), the cells were subjected to separation by fluorescence activated cell sorting (FACS). Genomic DNA was isolated from the FACS sorted Annexin-V positive/PI negative or the TUNEL positive cells and the ribozyme genes were then rescued by PCR amplification of the DNA.

Ribozyme genes were rescued from the FACS selected cell population by PCR rescue, which was performed on five separate aliquots of 1 μg of genomic DNA extracted from the cells using the QIAmp Blood Kit (Qiagen, Valencia, Calif.). PCR was carried out using the AmpliTaq Gold system (Perkin-Elmer, Norwalk, Conn.) with an initial denaturation at 94° C. for 10 min. followed by 35 cycles of 94° C. for 20 sec., 65° C. for 30 sec., and 72° C. for 30 sec. A final extension was performed at 72° C. for 7 min. PCR primers, 5′-GGCGGGACTATGGTTGCTGACTAAT-3′ (SEQ ID NO: 14) and 5′-GGTTATCACGTTCGCCTCACACGC-3′ (SEQ ID NO: 15) annealing within the vector amplified a 300 bp fragment containing the ribozyme genes. The pooled PCR product, which contained a pool of ribozyme genes, was isolated by electrophoresis on 1% agarose, purified using a Gel Extraction Kit (Qiagen), then digested with BamHI and MluI and ligated into vector pLPR digested with the same enzymes. The ligated DNA was used to transform DH12S E. coli bacteria by electroporation. The entire bacterial culture was plated on LB-agar plates containing ampicillin and incubated at 37° C. overnight. The resulting bacterial colonies were pooled and purified DNA was used in a triple transfection protocol (as described above in Example 1) to produce retroviral vector. Individual colonies were also sequenced by the standard dideoxy method using a vector primer 5′-CTGACTCCATCGAGCCAGTGTAGAG-3′ (SEQ ID NO: 16).

Three rounds of successive vector transduction, apoptosis induction, FACS selection and ribozyme gene rescue were performed. The third round of FACS selection showed that approximately 5-6% of the pLPR-library transduced cells had entered apoptosis as compared to 1-2% for the control vector. These results indicate that progressive selection of library transduced cells enriched the ribozyme pool for ribozymes that facilitate apoptosis.

Analysis of the ribozyme gene inserts following the third round of PCR rescue indicated enrichment of several ribozyme substrate binding sequences. Five predominant ribozyme substrate binding sequences designated RAP2 (SEQ ID NO: 17), RAP4 (SEQ ID NO: 18), RAP6 (SEQ ID NO: 19), RAP10 (SEQ ID NO: 20) and RAP594 (SEQ ID NO: 21) were identified. The substrate binding sequences of each of these identified ribozymes is listed in the first row of Tables 1-5.

TABLE 1

RAP2 Substrate Binding
Sequence and complementary RST

| | | |
|---|---|---|
| RAP2: substrate | 5′- CCAGTCCA | (SEQ ID NO: 17) |
| binding sequence | AGAA GACC -3′ | |
| RAP2: | 5′- GGTC NGTC | (SEQ ID NO: 22) |
| complementary RST | TGGACTGG -3′ | |

TABLE 2

RAP4 Substrate Binding
Sequence and complementary RST

| | | |
|---|---|---|
| RAP4: substrate | 5′- TCGTTGTG | (SEQ ID NO: 18) |
| binding sequence | AGAA AGCC -3′ | |
| RAP4: | 5′- GGCT NGTC | (SEQ ID NO: 23) |
| complementary RST | CACAACGA -3′ | |

TABLE 3

RAP6 Substrate Binding
Sequence and complementary RST

| | | |
|---|---|---|
| RAP6: Substrate | 5′- GTCTTCAT | (SEQ ID NO: 19) |
| binding sequence | AGAA GGCC -3′ | |
| RAP6: | 5′- GGCC NGTC | (SEQ ID NO: 24) |
| Complementary RST | ATGAAGAC -3′ | |

TABLE 4!RAP 10 Substrate Binding? ? !Sequence and complementary RST

| | | |
|---|---|---|
| RAP 10: Substrate | 5′- TGATCCGT | (SEQ ID NO: 20) |
| binding sequence | AGAA CATA -3′ | |
| RAP 10: | 5′- TATG NGTC | (SEQ ID NO: 25) |
| Complementary RST | ACGGATCA -3′ | |

TABLE 5

RAP594 Substrate Binding
Sequence and complementary RST

| | | |
|---|---|---|
| RAP594: Substrate | 5′- TATGCTGT | (SEQ ID NO: 21) |
| binding sequence | AGAA ATAA -3′ | |
| RAP594: | 5′- TTAT NGTC | (SEQ ID NO: 26) |
| Complementary RST | ACAGCATA -3′ | |

The validity of individual library-selected ribozymes for facilitating apoptosis induction was determined by transfection of DLD-1 cells followed by analysis of apoptosis induction. For this purpose, nucleic acid sequence encoding ribozymes having the RAP sequences shown in Tables 1-5 were cloned into pLPR in place of the 1 kb stuffer. Vector LPR-TL3 was used as a control.

For transfection, DLD-1 cells were grown to about 70% confluency in T75 flasks (about 5×10⁶ cells). The media was then removed and replaced with 0.8 ml of serum free Opti-MEM media (GIBCO). The cells were incubated for four hours at 37° C. with complexes containing a lipid-plasmid DNA complex.

The lipid-plasmid DNA complex was prepared by combining lipid reagent lipofectamine (GIBCO) at a ratio of 4 microliters lipid reagent to 1 microgram DNA (single ribozyme encoding or control LPR-TL3). Lipid/DNA complexes were allowed to form for 20 min. at room temperature before use.

After incubation of the cells with the complexes, the transfection medium was removed by aspiration and replaced with complete growth medium. The cells were cultured for 24 hrs before selection in growth medium containing puromycin (2 μg/ml). The next day, cells were re-fed with media containing 2 μg/ml puromycin. The cells were allowed to recover and expand for two weeks.

The cells were then tested for the ability to undergo apoptosis upon induction with CD95 ligating antibody. In all cases, transfection with the vector encoding ribozymes with the individual library-selected substrate binding sequences confirmed the previously observed phenotype—an increase in induction of apoptosis (10-20% of the transfected/selected DLD-1 cell population).

### Example 3

### Identification of Genes Involved in Inhibition of Apoptosis Induction

This example describes methods to identify cellular genes involved in inhibiting cells to the induction of apoptosis by the CD95 antibody. Since ribozymes recognize their cognate targets by sequence complementarity, the substrate binding sequence of a ribozyme which is associated with a particular phenotype can be used to define a ribozyme sequence tag (RST) that is present in a target gene involved in the phenotype. In the ribozyme library used herein, the RST is 16 bases long, comprising the two target binding arms (helix 1 and 2) surrounding the requisite NGUC in the target (see FIG. 1).

The second row of Tables. 1-5 above show the complementary RST (SEQ ID NOS: 22-26) for each library-derived substrate binding sequence. In each case, the first four bases (5′ end) representing the Helix 2 sequence and the last eight bases representing the Helix 1 sequence are the direct Watson-Crick base complement to the corresponding substrate binding sequence. The base at the fifth position from the 5′ end of the RST need not be specified and is shown as an “N.” The three bases located 3′ to the ‘N’ in the RSTs represent the gene sequence GTC, which following transcription, becomes the requisite cognate sequence GUC, recognized by “GUC ribozymes” (see FIG. 1).

To identify genes involved in inhibition to apoptosis induction by CD95 antibody, the gene and expressed sequence tag (EST) public databases were searched using the Basic Local Alignment Search Tool (“BLAST”) (http://www.ncbi.nlm.nih.gov/BLAST/) for the presence of the RSTs shown in Tables 1-5. The parameters of the BLAST search were “word size”=7 and “expected”=1,000. Four searches were done for each RST using A, T, C, or G in the “N” of the NGUC sequence of the RST.

BLAST searching of the RAP4-RST and RAP10-RST did not yield any substantive results (only incomplete matches with non-human sequences). In contrast, the BLAST search of the RAP2-RST and the RAP6-RST identified several completely matching sequences in the public databases, and the BLAST search of the RAP594-RST identified two separate 15/16 nucleotide matches.

The RAP6-RST perfectly matched a sequence within the NHMCZF gene (GenBank Accession No. AL096880) (SEQ ID NO: 27) located in the nr (non-redundant) database. The NHMCZF gene (entitled “Novel Human mRNA Containing Zinc Finger CH2 Domains”) has a high degree of identity to the mouse MAZR (SEQ ID NO: 28) and human PATZ (SEQ ID NO: 29) gene sequences, also located in the nr database. The RAP6-RST also perfectly matched a sequence within the EST gi:874139 (GenBank Accession No. H09317) (SEQ ID No: 30), referred to hereinafter as EST6.

The RAP2-RST was found to perfectly match the a sequence within EST fragment yf56a06.r1 (GenBank Accession No. R12420) (SEQ ID NO: 31), referred to hereinafter as EST2. Subsequent BLAST searches yielded perfect matches to sequences within eight other EST fragments:

602318810F1 (GenBank Accession No. BG116747) (SEQ ID NO: 32);

602317343F1 (GenBank Accession No. BG115920) (SEQ ID NO: 33);

602281666F1 (GenBank Accession No. BG111236) (SEQ ID NO: 34);

602248984F1 (GenBank Accession No. BF692624) (SEQ ID NO: 35);

601434123F1 (GenBank Accession No. BE892951) (SEQ ID NO: 36);

DKFZp761o0715 (GenBank Accession No. AL138059) (SEQ ID NO: 37);

cr22e03 (GenBank Accession No. AI754258) (SEQ ID NO: 38); and

zw05h03 (GenBank Accession No. AA495929) (SEQ ID NO: 39).

As described below in Example 6, it was subsequently determined that EST2 and the eight additional EST fragments described above are overlapping fragments of the putative cDNA FLJ22165 (GenBank Accession No. AK025818 (SEQ ID NO: 40)).

The RAP594-RST-matched 15/16 nucleotides of two gene sequences in the nr database. One of the matches was to CSNK2A1 (GENBANK Accession No. NM_{—}001895.1) (SEQ ID NO: 41) and the other was to FAPP2 (GENBANK Accession No. NM_{—}032639) (SEQ ID NO: 42).

The involvement of the NHMCZF gene (SEQ ID NO: 27) in inhibiting apoptosis induction with CD95 ligating antibody was confirmed as follows: The nucleotide sequence of the NHMCZF gene was inspected to determine if other segments of the gene might serve as additional RST sites, and “validation ribozymes” having substrate binding sites complementary to six of these putative RSTs were engineered, as listed in Table 6 below.

TABLE 6

Validation Ribozyme Substrate
Binding Sequences for NHMCZF Gene

| Validation | Ribozyme Substrate Binding Sequence |
|---|---|
| Ribozyme | (Based on the NHMCZF Gene) |
| NHMCZF-1 | 5′-AGCAGCCA AGAA GGCC-3′ |
| | (SEQ ID NO: 43) |
| NHMCZF-2 | 5′-TGAACACC AGAA CAAA-3′ |
| | (SEQ ID NO: 44) |
| NHMCZF-3 | 5′-ATCAGCCG AGAA CCCG-3′ |
| | (SEQ ID NO: 45) |
| NHMCZF-4 | 5′-CCCCATCA AGAA CCAT-3′ |
| | (SEQ ID NO: 46) |
| NHMCZF-5 | 5′-ACCCAAAG AGAA CGAG-3′ |
| | (SEQ ID NO: 47) |
| NHMCZF-6 | 5′-CAAATGCC AGAA GAAC-3′ |
| | (SEQ ID NO: 48) |

Retroviral expression plasmids encoding ribozymes having the substrate binding sequences shown in Table 6 were then generated, and DLD-1 cells were tested for CD95 apoptosis induction following transfection with the vectors, as described in Examples 1 and 2. One of these, NHMCZF-4 (SEQ ID NO: 46), whose complementary RST is ATGGAGTCTGATGGGG (SEQ ID NO: 49), bestowed the phenotype of facilitating induction of apoptosis by CD95 ligating antibody. This confirmed the original finding based upon RAP6 that the NHMZCF gene was involved in the inhibition of apoptosis.

Additional RSTs for the NHMZCF gene and the complementary ribozyme substrate binding sites are provided in Table 7 below:

TABLE 7

Additional Ribozyme Substrate Binding
Sequences and Target RSTs for NHMZCF

| Ribozyme Substrate | NHMZCF |
|---|---|
| Binding Sequence (5′-3′) | Target RST (5′-3′) |
| GTACGTTG AGAA GTTT | AAAC AGTC CAACGTAC |
| (SEQ ID NO: 50) | (SEQ ID NO: 73) |
| CCCCTGGG AGAA CAAA | TTTG GGTC CCCAGGGG |
| (SEQ ID NO: 51) | (SEQ ID NO: 74) |
| ACCACATA AGAA GCAT | ATGC GGTC TATGTGGT |
| (SEQ ID NO: 52) | (SEQ ID NO: 75) |
| AGGCTGGT AGAA CCGT | ACGG TGTC ACCAGCCT |
| (SEQ ID NO: 53) | (SEQ ID NO: 76) |
| CAGAGTGG AGAA GCTT | AAGC TGTC CCACTCTG |
| (SEQ ID NO: 54) | (SEQ ID NO: 77) |
| CATCATGG AGAA GCAC | GTGC GGTC CCATGATG |
| (SEQ ID NO: 55) | (SEQ ID NO: 78) |
| TGCCCACG AGAA CATG | GATG GGTC CGTGGGCA |
| (SEQ ID NO: 56) | (SEQ ID NO: 79) |
| GCAGGTCT AGAA CTTG | CAAG TGTC AGACCTGC |
| (SEQ ID NO: 57) | (SEQ ID NO: 80) |
| GGAGCGCA AGAA GTCT | AGAC CGTC TGCGCTCC |
| (SEQ ID NO: 58) | (SEQ ID NO: 81) |
| TTTTTGGG AGAA GCCA | TGGC AGTC CCCAAAAA |
| (SEQ ID NO: 59) | (SEQ ID NO: 82) |
| CGAGGAGA AGAA TGTT | AACA TGTC TCTCCTCG |
| (SEQ ID NO: 60) | (SEQ ID NO: 83) |
| CTAAAGAT AGAA CAAA | TTTG CGTC ATCTTTAG |
| (SEQ ID NO: 61) | (SEQ ID NO: 84) |
| TCCGTGGG AGAA CAGC | GCTG TGTC CCCACGGA |
| (SEQ ID NO: 62) | (SEQ ID NO: 85) |
| TTTCTAAA AGAA ATTG | CAAT GGTC TTTAGAAA |
| (SEQ ID NO: 63) | (SEQ ID NO: 86) |
| GAAACTGG AGAA GGTT | AACC AGTC CCAGTTTC |
| (SEQ ID NO: 64) | (SEQ ID NO: 87) |
| TGGGAGGG AGAA AAAA | TTTT GGTC CCCTCCCA |
| (SEQ ID NO: 65) | (SEQ ID NO: 88) |
| GGGAGGAT AGAA AACT | AGTT CGTCA TCCTCCC |
| (SEQ ID NO: 66) | (SEQ ID NO: 89) |
| TAGGTGGA AGAA CTAG | CTAG GGTC TCCACCTA |
| (SEQ ID NO: 67) | (SEQ ID NO: 90) |
| TCTTGGAG AGAA CTCA | TGAG TGTC CTCCAAGA |
| (SEQ ID NO: 68) | (SEQ ID NO: 91) |
| TAATGTGT AGAA GCGG | CCGC AGTC ACACATTA |
| (SEQ ID NO: 69) | (SEQ ID NO: 92) |
| CCTGACCA AGAA GGCA | TGCC AGTC TGGTCAGG |
| (SEQ ID NO: 70) | (SEQ ID NO: 93) |
| ACTTCCCT AGAA AGAC | GTCT GGTC AGGGAAGT |
| (SEQ ID NO: 71) | (SEQ ID NO: 94) |
| TCACATGT AGAA CAAC | GTTG TGTC ACATGTGA |
| (SEQ ID NO: 72) | (SEQ ID NO: 95) |

To confirm the involvement of the EST2 clone (SEQ ID NO: 31) in the inhibition of apoptosis, “validation ribozymes” were engineered, having the substrate binding sequences listed in Table 8 below:

TABLE 8

Validation Ribozyme Sequences for EST2

| Validation | |
|---|---|
| ribozyme | Substrate Binding Sequence |
| Est2-1 | 5′-TCCTCCCC AGAA CCCT-3′ |
| | (SEQ ID NO: 96) |
| Est2-2 | 5′-TCCAGACA AGAA AGCT-3′ |
| | (SEQ ID NO: 97) |

Retroviral expression plasmids encoding ribozymes having the substrate binding sequences shown in Table 8 were then generated, and DLD-1 cells were tested for CD95 triggered apoptosis induction following transfection with the plasmids, as described in Examples 1 and 2 above. Both “validation ribozymes” bestowed the phenotype of facilitating apoptosis induction. Their complementary RST sequences are SEQ ID NOS: 98 and 99, respectively. This confirmed the original finding based upon RAP2 that EST2 was involved in apoptosis inhibition.

Additional RSTs for the EST2 gene and the complementary ribozyme substrate binding sites, based upon the overlap with FLJ22165 (SEQ ID NO: 40) are provided in Table 9 below:

TABLE 9

Ribozyme Substrate Binding Sequences
and Target RSTs for cDNA fragment FLJ22165

| Ribozyme Substrate | FLJ22165 |
|---|---|
| Binding Sequence (5′-3′) | Target RST (5′-3′) |
| CTGACAAA AGAA GTCT | AGAC AGTC TTTGTCAG |
| (SEQ ID NO: 100) | (SEQ ID NO: 113) |
| GTAATTCT AGAA AAGA | TCTT TGTC AGAATTAC |
| (SEQ ID NO: 101) | (SEQ ID NO: 114) |
| TGTATTGA AGAA GAAA | TTTC TGTC TCAATACA |
| (SEQ ID NO: 102) | (SEQ ID NO: 115) |
| CCACATAA AGAA GGAA | TTCC TGTC TTATGTGG |
| (SEQ ID NO: 103) | (SEQ ID NO: 116) |
| CAAGCCCA AGAA AAAA | TTTT TGTC TGGGCTTG |
| (SEQ ID NO: 104) | (SEQ ID NO: 117) |
| CACTGCTA AGAA AGCC | GGCT TGTC TAGCAGTG |
| (SEQ ID NO: 105) | (SEQ ID NO: 118) |
| AGGCAACA AGAA AAAT | ATTT AGTC TGTTGCCT |
| (SEQ ID NO: 106) | (SEQ ID NO: 119) |
| CAACCTGT AGAA AGAG | CTCT TGTC ACAGGTTG |
| (SEQ ID NO: 107) | (SEQ ID NO: 120) |
| AGTCCAAT AGAA GCCA | TGGC TGTC ATTGGACT |
| (SEQ ID NO: 108) | (SEQ ID NO: 121) |
| TTTCCTGA AGAA CTTG | CAAG AGTC TCAGGAAA |
| (SEQ ID NO: 109) | (SEQ ID NO: 122) |
| CCTCAAGA AGAA CACC | GGTG GGTC TCTTGAGG |
| (SEQ ID NO: 110) | (SEQ ID NO: 123) |
| TTAGTAGA AGAA GGGT | ACCC TGTC TCTACTAA |
| (SEQ ID NO: 111) | (SEQ ID NO: 124) |
| AGTGCAGT AGAA CGAT | ATCG TGTC ACTGCACT |
| (SEQ ID NO: 112) | (SEQ ID NO: 125) |

Similarly, to confirm the involvement of the EST6 clone in apoptosis inhibition, “validation ribozymes” having the substrate binding sequences listed in Table 10 below were engineered against EST6, and DLD-1 cells were transfected with retroviral expression plasmids encoding for these ribozymes. However, in this case, neither of the “validation ribozymes” bestowed the phenotype of facilitating apoptosis induction, and the involvement of EST6 in this process was not confirmed.

TABLE 10

Validation Ribozyme Sequences for EST6

| Validation | |
|---|---|
| ribozyme | Substrate Binding Sequence |
| Est6-1 | 5′-ATACCCCT AGAA CTGA-3′ |
| | (SEQ ID NO: 126) |
| Est6-3 | 5′-ACATGTAG AGAA CGCA-3′ |
| | (SEQ ID NO: 127) |

In order to determine which of the two BLAST matches to RAP594—CSNK2A1 (SEQ ID NO: 41) or FAPP2 (SEQ ID NO: 42) was the correct match, “validation ribozymes” having the substrate binding sequences listed in Table 11 below were engineered against CSNK2A1, and “validation ribozymes” having the substrate binding sequences listed in Table 12 below were engineered against FAPP2:

TABLE 11

Validation Ribozyme sequences for CSNK2A1

| Validation | |
|---|---|
| Ribozyme | Substrate Binding Sequence |
| HRVG-TV130 | GGTTGGCG AGAA AAGC (SEQ ID NO: 128) |
| HRVG-TV 229 | CCACATGT AGAA CGTA (SEQ ID NO: 129) |
| HRVG-TV 463 | GGGTTCGT AGAA CAGG (SEQ ID NO: 130) |
| HRVG-TV 1001 | TCACTGTG AGAA AAGC (SEQ ID NO: 131) |
| HRVG-TV 1711 | AAGTGTGG AGAA GTGG (SEQ ID NO: 132) |
| HRVG-TV2094 | AGGGAAAA AGAA AAGG (SEQ ID NO: 133) |

TABLE 12

Validation Ribozyme Sequences for FAPP2

| Validation | |
|---|---|
| Ribozyme | Substrate Binding Sequence |
| FA5-VR1 | ATTTCACA AGAA GCCA (SEQ ID NO: 134) |
| FA5-VR2 | CAGAATTG AGAA CACC (SEQ ID NO: 135) |
| FA5-VR3 | TCTTGCTG AGAA TGAG (SEQ ID NO: 136) |
| FA5-VR4 | AAATTTGA AGAATCTC (SEQ ID NO: 137) |
| FA5-VR5 | TTAGATTT AGAA ACTT (SEQ ID NO: 138) |
| FA5-VR6 | TCCAGTTT AGAATTGG (SEQ ID NO: 139) |

DLD-1 cells were then transfected with retroviral expression plasmids encoding for these ribozymes, and the cells were assayed for their ability to undergo Fas-mediated apoptosis. None of the target validation ribozymes listed in Table 11 was able to confer sensitivity to Fas-mediated apoptosis in DLD-1 cells. However, both FA5-VR1 and FA5-VR5 were able to cause the DLD-1 to undergo apoptosis after induction by Fas. The complementary RST sequences of these two validation ribozymes are SEQ ID NOS: 140 and 141, respectively. This confirmed that the FAPP2 gene was the target of RAP594 and that RAP594 was involved in apoptosis inhibition.

### Example 4

### Confirmation of Target Knockdown

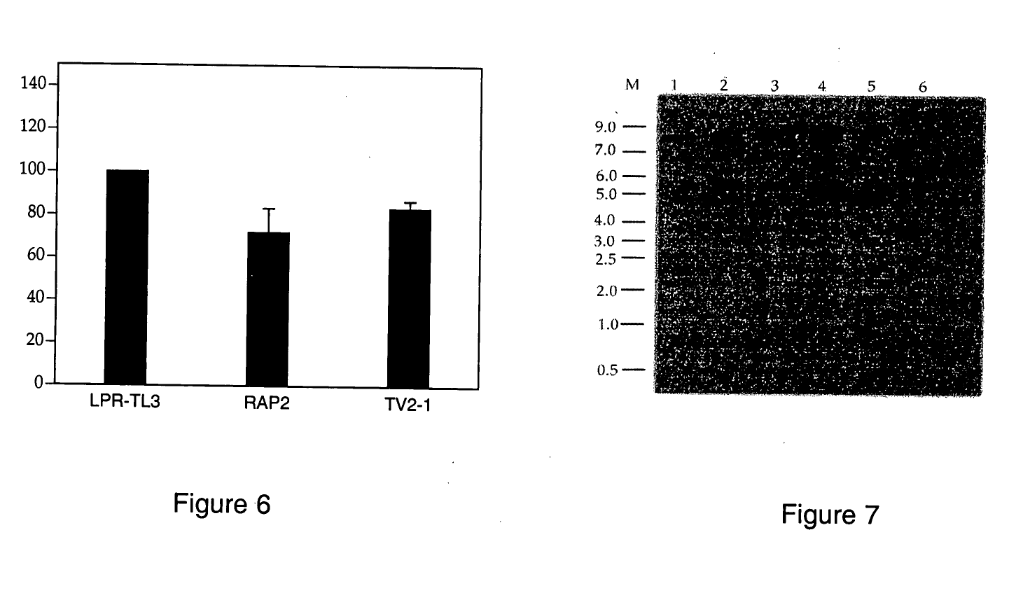

This example describes a method for confirming knockdown, or decrease in the level, of an RNA target identified by the methods described in the previous examples. DLD-1 cells were transfected with either a control plasmid (LPR-TL3) or retroviral plasmids expressing the RAP2 or EST2-1 ribozyme genes. Transfections and selection in puromycin were carried out as described above. Total RNA was extracted from the cells using the RNEASY kit (Qiagen). The RNA was analyzed by TaqMan real time RT-PCR, with the EST2 sequence used as the template to design the TaqMan probe (SEQ ID NO: 142) and primer set (SEQ ID NOS: 143 and 144). Five μg of total RNA from the cellular samples was used for the analysis. LPR-TL3 was used as the control. The results showed that the RAP2 ribozyme (SEQ ID NO: 17) and the “validation ribozyme” RTV2-1, also known as TV 2-1 or the Est2-1 validation ribozyme (SEQ ID NO: 96), caused a significant knockdown, or decrease in the mRNA, of the EST2 target gene, a 30% decrease using the RAP2 ribozyme (SEQ ID NO: 17) and the a 20% decrease using the Est2-1 (SEQ ID NO: 96) (see FIG. 6).

### Example 5

### Confirmation of Cellular Expression of RNA Target

This example describes a method for confirming that a partial gene sequence identified in Example 3 above, EST2 (SEQ ID NO: 31), is part of a larger mRNA that is normally expressed both in both tumor cell lines and normal tissue. Messenger RNA was prepared from five colon carcinoma cell lines: DLD-1; SW480; HT-29; Colo 220; SW1417. The RNA was prepared using the RNEASY kit and oligo dT (Qiagen). Messenger RNA from normal colon tissue was purchased (ResGen/Invitrogen) and prepared. One μg of mRNA was loaded onto a 1% agarose gel for each sample. Northern blot and radioactive probing of the blot was done by protocols known to those skilled in the art. The probe was generated by PCR using EST2 (SEQ ID NO: 31) as the template and primers TV2-R (SEQ ID NO: 143) and EST2ProbeF (SEQ ID NO: 145). The blot was able to detect a single band, approximately 7-7.5 kb in length, present in both the cell lines and the normal colon tissue (see FIG. 7). This indicates that the EST2 (SEQ ID NO: 31) sequence is part of a larger mRNA that is expressed both in tumor cell lines and normal tissue.

### Example 6

### Isolation, Assembly and Sequence of a Full-Length cDNA for EST2 Gene

This example describes the process of assembling the full-length cDNA for the gene that contains the EST2 fragment identified in Example 3 above. Utilizing the CAP contig assembly program (Indiana University Bioarchive), an initial contig was built from the overlapping cDNA FLJ22165 (SEQ ID NO: 40) and the 9 ESTs (SEQ ID NOS: 31-39) that matched the RAP2 RST (SEQ ID NO: 22). The size of this initial contig sequence (SEQ ID NO: 146) was approximately 1.7 kb. Using this contig fragment as the query sequence, a BLAST search was carried out which identified the overlapping EST sequence 601486342F1 (GenBank Accession No. BE877775) (SEQ ID NO: 147), and that extended the 5′ end of the contig to a total of about 2 kb (SEQ ID NO: 148).

With the above contig as a guide and template for primer design, RACE (Rapid Amplification of cDNA Ends) was undertaken to isolate and clone the full-length gene. The initial RACE protocol was performed on both colon and placental mRNA samples (ResGen/Invitrogen) using the SMART RACE kit (Clontech). The primer used to initiate the reverse transcriptase reaction was oligodT which was provided in the kit. The gene specific primer (5′-CACATCCCTCATTATAGTCAGAAAG-3′; SEQ ID NO: 149) annealed to nucleotides 738-762 in the 2 kb contig (SEQ ID NO: 148) and was used with the internal primer in the kit to perform the nested PCR. The RACE procedure was done as described in the manual provided with the kit. Nested PCR products were cloned into a TA Topo vector (Invitrogen) and analyzed by restriction enzyme (RE) digestion. Based on the sequence of the contig, a restriction enzyme map was constructed and prospective clones were digested with SpeI and NsiI separately. From these data it appeared that only the RACE reactions from the placental mRNA yielded the predicted clones. Clones with the predictive RE pattern were sequenced (Retrogen Inc.). The sequencing data revealed that the clones all overlapped with the 2 kb contig and actually extended the contig about 300 nucleotides at the 5′ end, thus yielding a new contig of about 2.3 kb (SEQ ID NO: 150). Because the full-length gene, predicted to be about 7 to about 7.5 kb in length (see Example 5 above) was not yet obtained, it was necessary to “walk down” the length of the mRNA to clone this gene.

The 2.3 kb contig (SEQ ID NO: 150) was then used as the query sequence for a BLAST search to see if the contig could be extended further. This searched yielded the EST fragment hv79F02 (GenBank Accession No. BE327693) (SEQ ID NO: 151) which overlapped the 2.3 kb contig and extended the sequence about 300 more bases at the 5′ end yielding a contig of about 2.6 kb (SEQ ID NO: 152). This contig was then used as the query sequence to search the human genome sequence at NCBI. The results of this search indicated that the contig hybridized with greater than 98% identity to a region tentatively assigned to chromosome 1.

A series of 10 primers were then designed based on the sequence about 3-5 kb upstream of the 5′ end of where the 2.6 kb contig hybridized on chromosome 1. These primers were utilized along with contig specific primers in PCR reactions of a RACE ready cDNA preparation of placental mRNA (Ambion). The PCR reactions from two of these primers (5′-TAACAATCCTTTGGAAGTCACTACTGG-3′; SEQ ID NO: 153; and 5′-AAGCCCAGCATTGCTAAGAGG-3′; SEQ ID NO: 154) gave products of predicted size and were subcloned into TA-TOPO vectors (Invitrogen) and sequenced. The sequencing data showed that these PCR products were overlapping with the sequence of the 2.6 kb contig and extended the contig at the 5′ end by about 800 bp, resulting in a gene fragment of a total of about 3.4 kb (SEQ ID NO: 155).

The 3.4 kb contig (SEQ ID NO: 155) was then used as the query sequence to search the proprietary transcript database of the Celera Genomics Group. This produced a hit with Celera transcript hCT 1782960 (SEQ ID NO: 156), which overlaps significantly with the 5′ region of the 3.4 kb contig (SEQ ID NO: 155) and extends the contig about 750 bp at the 5′ end, yielding a new contig of a total of about 4.1 kb (SEQ ID NO: 157).

To determine if the 750 bp described above indeed extended the gene, PCR experiments were undertaken using this 750 bp region of the 4.1 kb contig (SEQ ID NO: 157) as the template for primer design. When these primers (SEQ ID NOS: 159 and 160) were used in combination with contig specific primers (SEQ ID NOS: 161, 162 and 163), PCR products of the expected sizes were obtained. Sequencing confirmed that these PCR products were overlapping with the 3.4 kb contig (SEQ ID NO: 155) and that the new 750 bp at the 5′ end indeed extended the contig to about 4.1 kb.

The sequence of the 4.1 kb contig (SEQ ID NO: 157) contains an open reading frame (nucleotides 3-962) whose encoded protein (SEQ ID NO: 158) has significant identity with the hypothetical protein KIAA0456 (GENBANK Acession No: AB007925) (SEQ ID NO: 165), which is believed to be a GTPase activating protein. This suggests that the gene, which is shown herein to be involved in conferring resistance to Fas-induced apoptosis, may encode a GTPase activating protein.

The inability to find an mRNA of about 7 kb in the placental cDNA prompted the performance of a new Northern blot on mRNA from colon, brain and placental tissues, utilizing the same probe described above in Example 5. The results indicated that the gene containing EST2 had different sized mRNAs depending on the tissue in which it was expressed. The colon tissue contained an mRNA of about 7 kb; placenta had an mRNA of about 4.5 kb; and brain contained two species of the mRNA—a predominate band of about 5.5 kb and a minor band of about 7 kb. Thus, the RACE and contig assembly efforts described above were consistent with the size of the message present in placenta.

To determine whether the assembled contig represented a true messenger RNA, a lambda phage brain cDNA library (Human Brain Large-Insert cDNA Library, Clontech) was screened. This library was chosen because it contained both an approximately 5.5 kb mRNA and an approximately 7 kb mRNA, the latter being consistent with the size of the message present in colon tissue. The probe for these studies was a 500 bp NsiI fragment present in the 3′ end of the contig and lies about 50 bases upstream of the probe used for the Northern blots. From the chosen brain library, the 5.5 kb species of the mRNA was readily obtained and sequenced (SEQ ID NO: 166). This sequence of the 5.5 kb cDNA from the brain library contains the complete sequence of the 4.1 kb contig (SEQ ID NO: 157) and extends it at both the 5′ and 3′ ends of the sequence. The sequence that extends the 5′ end of the gene also extends the open reading frame that was present in the 4.1 kb contig (SEQ ID NO: 157), with further sequence identity to the GTPase protein described above. This further confirmed the identity of the protein product for this gene as a potential GTPase activating protein.

### Example 6

### In Vitro Efficacy

This example shows that ribozymes can treat cancer cells by making them more susceptible to apoptosis and more likely to respond to treatment.

Specifically, a bladder cancer cell line resistant to Fas was selected (regarding the role of Fas/Fas ligand system, including its role in cancer, see, for example, Gruss et al., J. Exp. Med.; 181:1235-38 (1995); Kagi et al., Science, 265:528-530 (1994); Nagata et al., Cell, 88:355-65 (1997); Runic, J. Clin. Endocrinol. Metab., 81:3119-22 (1996); Suda et al., Cell, 75:1169-78 (1993); and Perabo et al., Urology Oncology, 6:163-69 (2001)). These cells were transfected with one or both of the following ribozyme substrate binding sequences: 1) Tv2-2 (EST2-2 (SEQ ID NO: 97); and b) Sr6 (RAP6 (SEQ ID NO: 19). These binding sequences were each part of a synthetic, chimeric DNA/RNA hammerhead ribozyme structure (SEQ ID NOS: 6 and 5, respectively) that was used for the transfection. The specific cell line used was TCCSUP, which was derived from an anaplastic transitional cell carcinoma (TCC) in the neck of the urinary bladder of a 67 years old female.

These cells were transfected with either 1) Tv2-2; 2) SR6; 3) a “scrambled” control (identical structure to Tv2-2 or SR6 but with an additional non-specific sequence that cannot bind to any known gene); or 4) a combination of Tv2-2 and SR6 at a ratio of 1:1.

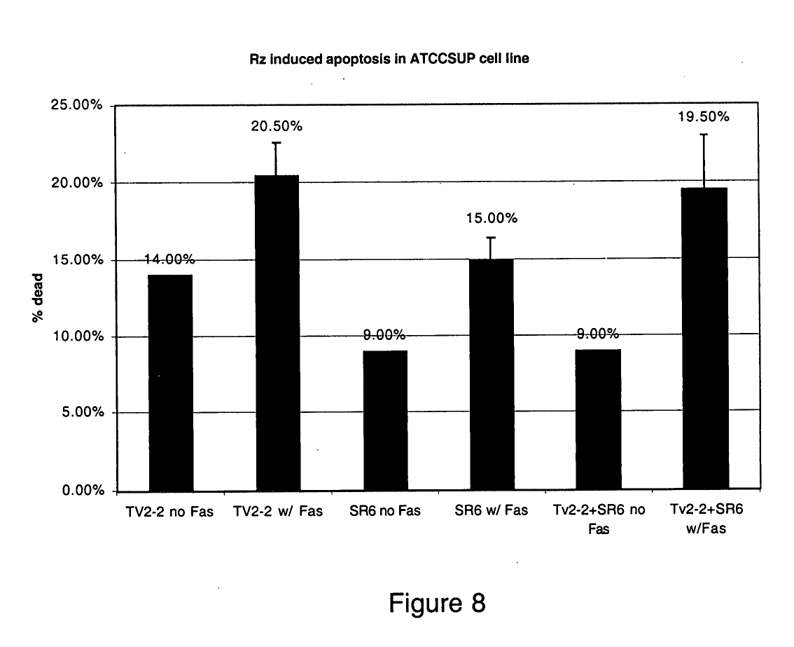

Cells were transfected accordingly to the manufacturer recommendations (GTS, CA-USA). In brief, cells were plated and 24 hours later, transfected using a Gene Porter kit with the different ribozymes. After 24 additional hours, cells were treated with Fas antibody. Eighteen hours later, cells were analyzed with Annexin V detection method. The results of these experiments are presented in FIG. 8.

As shown in FIG. 8, the presence of each ribozyme having the indicated substrate binding sequence, combined with Fas, dramatically increased the percentage of cells that underwent apoptosis, as compared to the presence of these ribozymes without Fas. Moreover, the combination of both ribozymes with Fas more than doubled the percentage of cell undergoing apoptosis. Thus, this example demonstrates that Tv2-2 and SR6 were able to lift the cell resistance to Fas and therefore make them susceptible to apoptosis and, accordingly, more likely to respond to treatment.

All references made herein, including journal articles, patent applications, patents and other publications, are incorporated by reference in their entirety.

We claim:

1. An isolated molecule comprising bases 2 to 8 of SEQ ID NO: 19 and also comprising bases 13 to 16 of SEQ ID NO: 19, wherein one or more of said bases, which are each at the 1-position of a sugar moiety, independently and optionally have —OH, —OC₁₋₆Alkyl, halo, amino, azido, nitro or phenyl at the 2-position of said sugar moiety, and wherein one or more of said bases are optionally further modified to result in a modified base.

2. An isolated molecule comprising bases 2 to 8 of SEQ ID NO: 97 and also comprising bases 13 to 16 of SEQ ID NO: 97, wherein one or more of said bases, which are each at the 1-position of a sugar moiety, independently and optionally have —OH, —OC₁₋₆Alkyl, halo, amino, azido, nitro or phenyl at the 2-position of said sugar moiety, and wherein one or more of said bases are optionally further modified to result in a modified base.

3. The molecule of claims 1 or 2, wherein said bases are not further modified.

4. The molecule of claim 3, wherein bases 2, 4 and 5 of SEQ ID NO: 19 are each thymine, and base 16 of SEQ ID NO: 97 is uracil.

5. The molecule of claims 1 or 2, further comprising bases 8 to 11 of SEQ ID NO: 5.

6. The molecule of claim 4, further comprising bases 8 to 15 of SEQ ID NO: 5.

7. The molecule of claims 1 or 2, wherein at least one but not all of the 2-positions of said sugar moieties have —OH, —OC₁₋₆Alkyl, halo, amino, azido, nitro or phenyl.

8. A composition comprising the molecule of claim 1 and the molecule of claim 2, wherein said bases are not optionally further modified.

9. The molecule of claim 4, wherein the 2-position of the sugar moiety of each of bases 6 and 7 is —OCH₃.

10. The molecule of claim 4, comprising SEQ ID NO: 5.

11. The molecule of claim 4, comprising SEQ ID NO: 6.

12. The molecule of claim 4, consisting of SEQ ID NO: 5.

13. The molecule of claim 4, consisting of SEQ ID NO: 6.

14. A hammerhead ribozyme, comprising the molecule of claims 5 or 7.

15. A method of facilitating the induction of apoptosis in a cell, comprising introducing in said cell the molecule of claim 1.

16. The method of claim 15, wherein said cell is resistant to apoptosis.

17. The method of claim 16, wherein said cell is a cancer cell.

18. The method of claim 17, further comprising contacting said cell with an apoptosis inducing agent.

19. The method of claim 18, wherein said agent is Fas.

20. A method of facilitating the induction of apoptosis in a cell, comprising introducing in said cell the molecule of claim 2.

21. The method of claim 20, wherein said cell is resistant to apoptosis.

22. The method of claim 21, wherein said cell is a cancer cell.

23. The method of claim 22, further comprising contacting said cell with an apoptosis inducing agent.

24. The method of claim 23, wherein said agent is Fas.

25. A method of facilitating the induction of apoptosis in a cell, comprising introducing in said cell the molecules of claims 1 and 2.

26. The method of claim 25, wherein said cell is resistant to apoptosis.

27. The method of claim 26, wherein said cell is a cancer cell.

28. The method of claim 27, further comprising contacting said cell with an apoptosis inducing agent.

29. The method of claim 28, wherein said agent is Fas.

30. An isolated compound comprising SEQ ID NO: 158 or an amino acid sequence with 98% or more identity with SEQ ID NO: 158.

31. An isolated compound comprising amino acids 1 to 247 of SEQ ID NO: 167 or an amino acid sequence with 98% or more identity with amino acids 1 to 247 of SEQ ID NO: 167.

32. The isolated compound of claim 31, comprising SEQ ID NO: 167 or an amino acid sequence with 98% or more identity with SEQ ID NO: 167.

33. A method of identifying a molecule that promotes induction of apoptosis in a cell, comprising:
a. contacting said molecule with the a compound comprising 10 or more contiguous amino acids of SEQ ID NO: 158 or 10 or more contiguous amino acids of amino acids 1 to 247 of SEQ ID NO: 167, wherein said molecule binds said compound;
b. introducing said molecule into a cell; and
c. measuring the level of apoptosis in said cell, where an increase in the level of apoptosis in said cell indicates that said molecule promotes induction of apoptosis.

34. An isolated molecule comprising SEQ ID NO: 146 or a molecule with 95% or more identity with SEQ ID NO: 146.

35. The isolated molecule of claim 34, comprising SEQ ID NO: 148 or a molecule with 95% or more identity with SEQ ID NO: 148.

36. The isolated molecule of claim 34, comprising SEQ ID NO: 150 or a molecule with 95% or more identity with SEQ ID NO: 150.

37. The isolated molecule of claim 34, comprising SEQ ID NO: 152 or a molecule with 95% or more identity with SEQ ID NO: 152.

38. The isolated molecule of claim 34, comprising SEQ ID NO: 155 or a molecule with 95% or more identity with SEQ ID NO: 155.

39. The isolated molecule of claim 34, comprising SEQ ID NO: 157 or a molecule with 95% or more identity with SEQ ID NO: 157.

40. The isolated molecule of claim 34, comprising SEQ ID NO: 166 or a molecule with 95% or more identity with SEQ ID NO: 166.
